(19) United States
(12) Patent Application Publication
(10) Pub. No.: US 2024/0050749 A1
(43) Pub. Date: Feb. 15, 2024

(54) OCCIPITAL NERVE STIMULATION FOR TREATMENT OF PAIN

(71) Applicant: SPR THERAPEUTICS, INC., Cleveland, OH (US)

(72) Inventors: Joseph W. Boggs, Chapel Hill, NC (US); Nathan D. CROSBY, Cleveland, OH (US); Amorn WONGSARNPIGOON, Chapel Hill, NC (US); Claire ZURN, Cleveland, OH (US); Matthew G. DEBOCK, Morrisville, NC (US); Meredith J. McGee, Cary, NC (US); Robert B. STROTHER, Willoughby Hills, OH (US)

(21) Appl. No.: 18/379,265

(22) Filed: Oct. 12, 2023

Related U.S. Application Data
(63) Continuation of application No. PCT/US2022/053922, filed on Dec. 23, 2022.
(60) Provisional application No. 63/293,123, filed on Dec. 23, 2021.
(60) Provisional application No. 63/346,382, filed on May 27, 2022.
(60) Provisional application No. 63/399,823, filed on Aug. 22, 2022.

(51) Int. Cl. A61N 1/36; A61N 1/06; A61N 1/20; A61N 1/05

(52) U.S. Cl. CPC A61N 1/36075; A61N 1/06; A61N 1/205; A61N 1/0558; A61N 1/3615; A61N 1/36171; A61N 1/36175; A61N 1/36178; A61N 1/0502

(57) ABSTRACT

Provided are systems and methods for treating pain in the head and neck, including headache pain. The systems and methods may be used to deliver electrical stimulation to the occipital nerve(s). The systems and methods may be used relieve pain in the region innervated by the targeted occipital nerves as well as one or more regions of referred pain that occur outside the innervation region of the occipital nerves. Due to the complex structures and anatomy around the occipital nerve(s), precise placement of the lead is achieved by the described systems and methods.

### CROSS REFERENCE TO RELATED APPLICATIONS

The present application claims priority to U.S. Patent Application No. 63/293,123, filed on Dec. 23, 2021, entitled “Occipital Nerve Stimulation for Treatment of Pain,” claims priority to U.S. Patent Application No. 63/399,823, filed on Aug. 22, 2022, entitled “Occipital Nerve Stimulation for Treatment of Pain,” and claims priority to U.S. Patent Application No. 63/346,382, filed on May 27, 2022, entitled “Nerve Stimulation for Treatment of Shoulder Pain,” all of which are each incorporated herein by reference in their entirety.

### FIELD OF INVENTION

The present disclosure generally relates to a system and method for treating pain in the head and neck, including headache pain. More particularly, the disclosure relates to a system and method of treating pain in the head and neck in patients by delivering electrical stimulation to the occipital nerve(s).

### BACKGROUND

The present disclosure generally relates to a system and method for treating pain in the head and neck, including headache pain and other pain associated with the occipital nerve. More particularly, the disclosure relates to a system and method of treating pain in the head and neck in patients by delivering electrical stimulation to the occipital nerve(s) and/or by selectively activating specific nerve fibers with an electrode situated proximate to, but not in direct contact with, portions of one or more of the occipital nerves.

Pain in the head and neck, especially in the region of the head and neck innervated by the occipital nerves and also including pain such as referred pain in other regions of the head and neck outside the direct innervation area of the occipital nerves, can be caused by a variety of injuries or conditions, such as occipital neuralgia, cervicogenic headache, occipital migraine, post-traumatic headache such as following traumatic brain injury (TBI), injury to certain structures in the neck or cervical spinal region, and other types of headache.

Existing treatments for headaches and head pain, such as acute, chronic, abortive, and preventive pain medications, interventional procedures such as injections, nerve ablations, and nerve blocks, and surgical procedures have limited efficacy and/or carry risks of complications, dependence, and debilitating side effects, and are often insufficient. Previous approaches to neurostimulation, including stimulation of the occipital nerves, for headaches and head pain have also been associated with practical and/or technical limitations.

Therefore, there is a need for an improved system and method to provide relief of pain in the head and neck, including headache pain.

### SUMMARY

The following presents a summary of this disclosure to provide a basic understanding of some aspects. This summary is intended to neither identify key or critical elements nor define any limitations of embodiments or claims. This summary may provide a simplified overview of some aspects that may be described in greater detail in other portions of this disclosure. Furthermore, any of the described aspects may be isolated or combined with other described aspects without limitation to the same effect as if they had been described separately and in every possible combination explicitly.

Provided is a method for relief of pain. The method may comprise inserting a percutaneous lead comprising at least one electrode into a posterior neck inferior to a nuchal ridge targeting at least one occipital nerve; positioning the at least one electrode a therapeutically effective distance from the at least one occipital nerve; stimulating the at least one occipital nerve through at least one electrode; and activating target large diameter fibers in the at least one occipital nerve while avoiding activation of non-target fibers, wherein the activation of target fibers produces relief of pain in a distribution of the at least one occipital nerve and in one or more region(s) of referred pain outside the distribution of the at least one occipital nerve by modulating activity at a point of convergence of the at least one occipital nerve and one or more nerve(s) innervating the region of referred pain.

In an embodiment, the percutaneous lead is inserted a length inside tissue to facilitate tissue ingrowth such that the percutaneous lead is resistant to migration during movement of a head and neck of a patient. In an embodiment, the percutaneous lead is an open coil lead. In an embodiment, the length is greater than or equal to 4 cm. In an embodiment, the length is at least three-times greater than a length of the at least one electrode. In an embodiment, the percutaneous lead comprises one or more anchoring structures. In an embodiment, the at least one electrode is positioned outside of the central nervous system. In an embodiment, the percutaneous lead is configured for insertion into a portion of a body that is proximal to the region of referred pain. In an embodiment, the electrical stimulation occurs proximal to the region of referred pain.

In an embodiment, the at least one occipital nerve is selected from a group consisting of: greater occipital nerve, lesser occipital nerve, third occipital nerve, C2 nerve, C3 nerve, C2 medial branch nerve, C3 medial branch nerve, cervical plexus. In an embodiment, the at least one nerve selected from the group includes at least one distal branch of the selected nerve or nerves. In an embodiment, the at least one electrode is positioned proximal to a point on the at least one occipital nerve at which one or more nerve fibers branch off from the at least one occipital nerve to innervate distal structures in a portion of a painful region innervated by the at least one occipital nerve. In an embodiment, the electrode is positioned along the at least one occipital nerve such that the comfortable sensations are generated in the distribution of the at least one occipital nerve. In an embodiment, the comfortable sensations are generated only in the portion of the painful region innervated by the at least one occipital nerve.

In an embodiment, the stimulation does not generate sensations in a distribution of the non-target fibers. In an embodiment, the stimulation does not generate sensations in the region(s) of referred pain. In an embodiment, the non-target fiber comprises a trigeminal nerve or a branch of the trigeminal nerve and the point of convergence is a trigeminocervical complex. In an embodiment, the region of referred pain is one or more of the parietal, temporal, frontal, frontotemporal, retroorbital, supraorbital, and auricular regions. In an embodiment, the electrical stimulation includes a first parameter selected from a group consisting of: frequency, pulse duration, amplitude, duty cycle, pattern of stimulus pulses, polarity, a predetermined number of phases, and waveform shape. In an embodiment, the electrical stimulation includes a second parameter selected from a group consisting of: frequency, pulse duration, amplitude, duty cycle, pattern of stimulus pulses, polarity, a predetermined number of phases, and waveform shape.

In an embodiment, the first parameter is amplitude and the second parameter is pulse duration. In an embodiment, an optimal amplitude and pulse duration are selected by increasing the amplitude until discomfort is produced, then decreasing the amplitude and correspondingly increasing the pulse duration to maximize activation of target fibers while avoiding activating non-target fibers. In an embodiment, the target large diameter fibers in the at least one occipital nerve are A-alpha and A-beta sensory afferent fibers. In an embodiment, the non-target fibers are one or more of the following: small diameter A-delta or C fibers in the at least one occipital nerve, small diameter A-delta or C fibers in the cutaneous, subcutaneous, or muscle tissue in the vicinity of the occipital nerve, or motor fibers in muscle tissue in the vicinity of the at least one occipital nerve. In an embodiment, the muscle tissue in the vicinity of the occipital nerve comprises one or more of the rotatores, semispinalis cervicis, semispinalis capitis, splenius cervicis, splenius capitis, multifidus, oblique capitis inferior, or trapezius muscles.

In an embodiment, the at least one electrode is formed integrally on a portion of the lead positioned spaced apart from a distal end of the percutaneous lead. In an embodiment, the distal end of the electrode is positioned outside of a therapeutically effective distance from the target nerve while the electrode is positioned within a therapeutically effective distance from the at least one occipital nerve. In an embodiment, the percutaneous lead is configured for insertion into the posterior neck inferior to the nuchal ridge targeting at least one occipital nerve at one or more of C2 lamina, C2 lateral articular pillar, C2/C3 joint, C3 lamina, C3 lateral articular pillar, or between obliquus capitis inferior muscle and semispinalis cervicis muscle. In an embodiment, an entry site of the percutaneous lead is at least 2 spinal levels inferior to an intended position of the at least one electrode to stimulate the target nerve and the percutaneous lead is inserted along a non-intersecting trajectory to achieve a therapeutically effective distance from the at least one occipital nerve. In an embodiment, the entry site is located inferior to a hairline. In an embodiment, wherein the entry site is at a midline and the percutaneous lead is inserted along a non-intersecting trajectory to achieve a therapeutically effective distance from the at least one occipital nerve.

Provided is a method to alleviate pain in a head and a neck. In an embodiment, the method may comprise: inserting a coiled lead into the neck inferior to the C1 vertebra, wherein the coiled lead: i) is configured to be inserted a length inside a body to facilitate tissue ingrowth along an implanted length of the coiled lead such that the coiled lead is resistant to migration during physiological movement of the head and the neck, ii) is configured with mechanical properties to withstand forces placed upon the coiled lead by stresses, motion, and movement of tissues in an area of occipital nerves; stimulating at least one occipital nerve through an electrode formed on the coiled lead and operatively coupled to an electrical stimulation device, wherein the electrode is positioned at a therapeutically effective distance from a portion of at least one nerve that innervates the area of the occipital nerves, and outside of an electrically activating distance from non-target nerve fibers in cutaneous tissue, subcutaneous tissue, or muscles proximate to the at least one occipital nerve; activating target fibers in the at least one occipital nerve while preventing activation of non-target fibers in the at least one occipital nerve and preventing activation of the non-target fibers in the cutaneous tissue, subcutaneous tissue, or muscles proximate to the at least one occipital nerve, wherein the activation of the target peripheral nerve fibers produces pain relief in a region of pain in a distribution of the at least one occipital nerve and in one or more region(s) of referred pain outside the distribution of the at least one occipital nerve by modulating central nervous system plasticity associated with the pain.

In an embodiment, the lead is deployable by an introducer needle. In an embodiment, the introducer needle is greater than or equal to 17 gauge and the coiled lead is configured to be deployed by the introducer needle. In an embodiment, the introducer needle is configured to be bent prior to its insertion in the body wherein the coiled lead is placed in a location that is not in an unobstructed straight line with an insertion site. In an embodiment, a maximum radius of curvature of the introducer needle is large enough whereby a lead path does not turn more than 90 degrees. In an embodiment, a length of the coiled lead to be inserted inside the body is greater than or equal to 4 cm. In an embodiment, a length to be inserted inside the body of the coiled lead is at least three-times greater than a length of the electrode. In an embodiment, the at least one occipital nerve is selected from a group consisting of: greater occipital nerve, lesser occipital nerve, third occipital nerve, C2 nerve, C3 nerve, C2 medial branch nerve, C3 medial branch nerve, cervical plexus. In an embodiment, the at least one non-targeted fibers is the trigeminal nerve or one or more branch(es) of the trigeminal nerve.

In an embodiment, the region of referred pain is one or more of the parietal, temporal, frontal, frontotemporal, retroorbital, supraorbital, and auricular regions. In an embodiment, the lead is configured to be inserted inferior to C1 targeting at least one occipital nerve at one or more of the C2 lamina, C2 lateral articular pillar, C2/C3 joint, C3 lamina, C3 lateral articular pillar, or in one or more muscles in a posterior of the neck. In an embodiment, the muscles include one or more of rotatores, multifidus, semispinalis cervicis, semispinalis capitis, splenius cervicis, splenius capitis, obliquus capitis inferior, or trapezius.

In an embodiment, an entry site of the coiled lead is at least one spinal level inferior to a target location. In an embodiment, the entry site is inferior to a level of a C4 vertebra. In an embodiment, the entry site is located inferior to a hairline facilitating placement of bandaging materials over an exit site of the coiled lead while avoiding placing bandaging materials at or superior to the hairline. In an embodiment, the coiled lead is inserted along a non-intersecting trajectory generally from inferior to superior to achieve the therapeutically effective distance from the at least one occipital nerve. In an embodiment, the entry site is at a midline and the coiled lead is inserted along a non-intersecting trajectory generally from medial to lateral to achieve the therapeutically effective distance from the at least one occipital nerve.

In an embodiment, the therapeutically effective distance is a remote location enabling preferential activation of the target fibers in the at least one occipital nerve while avoiding activation of the non-target fiber in the at least one occipital nerve. In an embodiment, the therapeutically effective distance is 0.5 to 3.0 cm In an embodiment, the target fibers in the at least one occipital nerve comprise large diameter sensory fiber. In an embodiment, the activation of the target fibers generates comfortable sensations in the distribution of the at least one occipital nerve. In an embodiment, the comfortable sensations are generated in the region of pain. In an embodiment, the comfortable sensations are not generated in the region of referred pain. In an embodiment, the non-target fibers in the at least one occipital nerve are small diameter fibers. In an embodiment, the non-target nerve fibers include small diameter fibers in cutaneous tissue layers, subcutaneous tissue layers, or muscle tissue in a vicinity of the at least one occipital nerve and the electrode. In an embodiment, the non-target fibers include motor fibers in one or more muscle(s) in a vicinity of the at least one occipital nerve and the electrode. In an embodiment, the muscles include one or more of rotatores, multifidus, semispinalis cervicis, semispinalis capitis, splenius cervicis, splenius capitis, obliquus capitis inferior, or trapezius.

In an embodiment, the electrical stimulation device is an external, body-worn pulse generator. In an embodiment, the electrical stimulation device is an implanted pulse generator In an embodiment, the electrical stimulation comprises a first parameter selected from a group consisting of: frequency, pulse duration, amplitude, duty cycle, pattern of stimulus pulses, polarity, a predetermined number of phases, and waveform shape. In an embodiment, the electrical stimulation comprises a second parameter selected from a group consisting of: frequency, pulse duration, amplitude, duty cycle, pattern of stimulus pulses, polarity, a predetermined number of phases, and waveform shape. In an embodiment, the first parameter is amplitude and the second parameter is pulse duration. In an embodiment, an optimal amplitude and pulse duration are selected by increasing the amplitude until discomfort is produced, then decreasing the amplitude and correspondingly increasing the pulse duration to maximize activation of target fibers innervating the region of pain without activating non-target fibers. In an embodiment, the modulation of central nervous system plasticity associated with chronic pain comprises expansion of non-painful cortical representations and/or contraction of painful cortical representations and/or the functional remapping of body regions in a somatosensory cortex.

Provided is a system for the relief of pain. In an embodiment, the system may comprise a percutaneous lead configured for insertion into a posterior neck inferior to a nuchal ridge targeting at least one occipital nerve; at least one electrode formed on the percutaneous lead configured to be positioned at a therapeutically effective distance from the at least one occipital nerve; and an electrical stimulation device operatively coupled to the percutaneous lead and configured to apply electrical stimulation through the at least one electrode to the at least one occipital nerve to provide relief of pain in a distribution of the at least one occipital nerve and in one or more region(s) of referred pain outside the distribution of the at least one occipital nerve by modulating activity at a point of convergence of the at least one occipital nerve and one or more non-targeted nerve(s) innervating the region of referred pain.

In an embodiment, the percutaneous lead is configured to be inserted a length inside a body to produce tissue ingrowth such that the percutaneous lead is resistant to migration during movement of a head and neck. In an embodiment, the percutaneous lead is an open coil lead. In an embodiment, the length to be inserted inside the body is greater than or equal to 4 cm. In an embodiment, the length is at least three-times greater than a length of the at least one electrode. In an embodiment, the percutaneous lead comprises one or more anchoring structures. In an embodiment, the at least one electrode is positioned outside of a central nervous system. In an embodiment, the percutaneous lead is configured for insertion into a portion of the body that is proximal to the region of pain.

In an embodiment, the electrical stimulation occurs proximal to the region of pain. In an embodiment, the at least one occipital nerve is selected from a group consisting of: greater occipital nerve, lesser occipital nerve, third occipital nerve, C2 nerve, C3 nerve, C2 medial branch nerve, C3 medial branch nerve, cervical plexus. In an embodiment, the at least one occipital nerve is selected from a group that comprises at least one distal branch of a selected nerve or nerves. In an embodiment, the at least one electrode is positioned proximal to a point on the at least one occipital nerve at which one or more nerve fibers branch off from the at least one occipital nerve to innervate distal structures in the region of pain. In an embodiment, the at least one electrode is positioned along the at least one occipital nerve wherein comfortable sensations are generated only in the region of pain or an area immediately surrounding the region of pain. In an embodiment, stimulation of the at least one occipital nerve creates comfortable sensations in a distribution of the at least one occipital nerve and does not generate sensations in a distribution of the non-targeted nerve that innervates the region of referred pain. In an embodiment, the at least one non-targeted nerve is a branch of a trigeminal nerve and the point of convergence is a trigeminocervical complex. In an embodiment, the region of referred pain is one or more of a parietal, temporal, frontal, frontotemporal, retroorbital, supraorbital, and auricular regions.

In an embodiment, the electrical stimulation comprises a first parameter selected from a group consisting of: frequency, pulse duration, amplitude, duty cycle, pattern of stimulus pulses, polarity, a predetermined number of phases, and waveform shape. In an embodiment, the electrical stimulation comprises a second parameter selected from a group consisting of: frequency, pulse duration, amplitude, duty cycle, pattern of stimulus pulses, polarity, a predetermined number of phases, and waveform shape. In an embodiment, the first parameter is amplitude and the second parameter is pulse duration. In an embodiment, an optimal amplitude and pulse duration are selected by increasing the amplitude until discomfort is produced, then decreasing the amplitude and correspondingly increasing the pulse duration to maximize activation of target fibers innervating the region of pain without activating non-target fibers.

In an embodiment, the stimulation activates target large diameter fibers in the at least one occipital nerve. In an embodiment, the stimulation prevents activation of non-target small diameter fibers in the at least one occipital nerve. In an embodiment, the stimulation prevents activation of non-target fibers in cutaneous tissue, subcutaneous tissue, and muscles proximate to the at least one occipital nerve. In an embodiment, the stimulation prevents activation of non-target fibers in rotatores, semispinalis cervicis, semispinalis capitis, splenius cervicis, splenius capitis, multifidus, oblique capitis inferior, or trapezius muscles.

In an embodiment, the at least one electrode is formed integrally at the distal end of the lead. In an embodiment, the at least one electrode is formed integrally on a portion of the lead not at the distal end. In an embodiment, the percutaneous lead is configured for insertion into the posterior neck inferior to the nuchal ridge targeting at least one occipital nerve at one or more of C2 lamina, C2 lateral articular pillar, C2/C3 joint, C3 lamina, C3 lateral articular pillar, or between the obliquus capitis inferior muscle and semispinalis cervicis muscle. In an embodiment, an entry site of the percutaneous lead is at least 2 spinal levels inferior to a target location and the percutaneous lead is inserted along a non-intersecting trajectory to achieve the therapeutically effective distance from the at least one occipital nerve. In an embodiment, the entry site is located inferior to a hairline to facilitate placement of bandaging materials over the entry site. In an embodiment, the entry site is at the midline and the percutaneous lead is inserted along a non-intersecting trajectory to achieve the therapeutically effective distance from the at least one occipital nerve.

Provided is a system to alleviate pain in a head and neck. In an embodiment, the system may comprise a coiled lead comprising a diameter facilitating percutaneous insertion into a neck inferior to the C1 vertebra, wherein the coiled lead: i) is configured to be inserted a length inside a body to facilitate tissue ingrowth such that the coiled lead is resistant to migration during movement of the head and neck, ii) is configured with mechanical properties to withstand forces placed upon the coiled lead by stresses, motion, and movement of tissues in an area of occipital nerves preventing mechanical failure; an electrode formed on the coiled lead, wherein the electrode is positioned at a therapeutically effective distance from a portion of at least one occipital nerve that innervates an occipital region, and outside of an electrically activating distance from non-target nerve fibers in cutaneous tissues, subcutaneous tissues, or muscles surrounding the at least one occipital nerve; and an electrical stimulation device operatively coupled to the coiled lead and configured to apply electrical stimulation through the electrode to the at least one occipital nerve to activate target peripheral nerve fibers while preventing activation of non-target peripheral nerve fibers in the at least one occipital nerve, wherein activation of target fibers produces relief of headache pain in a region(s) innervated by the at least one occipital nerve and in one or more region(s) of referred pain innervated by one or more non-target nerves by modulating central nervous system plasticity.

In an embodiment, the coiled lead comprises a diameter configured to be deployed by an introducer needle. In an embodiment, the introducer needle is greater than or equal to 17 gauge. In an embodiment, the introducer needle is configured to bend before insertion in the body such that the coiled lead is placed in a location that is not in an unobstructed straight line with an insertion site. In an embodiment, a maximum radius of curvature of the introducer needle is configured such that a lead path does not turn more than 90 degrees. In an embodiment, a length of the coiled lead configured to be inserted inside the body is greater than or equal to 4 cm. In an embodiment, a length of the coiled lead configured to be inserted inside the body is at least three-fold times than a length of the electrode. In an embodiment, the at least one occipital nerve is selected from a group consisting of: greater occipital nerve, lesser occipital nerve, third occipital nerve, C2 nerve, C3 nerve, C2 medial branch nerve, C3 medial branch nerve, cervical plexus. In an embodiment, the at least one occipital nerve is selected from a group comprising at least one distal branch of the selected nerve or nerves. In an embodiment, the at least one non-targeted nerve is the trigeminal nerve or one or more branch(es) of the trigeminal nerve. In an embodiment, the region of referred pain is one or more of the parietal, temporal, frontal, frontotemporal, retroorbital, supraorbital, and auricular regions.

In an embodiment, the coiled lead is configured to be inserted inferior to C1 targeting at least one occipital nerve at one or more of the C2 lamina, C2 lateral articular pillar, C2/C3 joint, C3 lamina, C3 lateral articular pillar, or in one or more muscles in a posterior neck. In an embodiment, the muscles include one or more of the rotatores, multifidus, semispinalis cervicis, semispinalis capitis, splenius cervicis, splenius capitis, obliquus capitis inferior, or trapezius. In an embodiment, an entry site of the coiled lead is at least one spinal level inferior to the target location. In an embodiment, the entry site is below C4. In an embodiment, the entry site is located inferior to a hairline. In an embodiment, the coiled lead is inserted along a non-intersecting trajectory generally from inferior to superior to achieve the therapeutically effective distance from the at least one occipital nerve. In an embodiment, the entry site is at the midline and the coiled lead is inserted along a non-intersecting trajectory generally from medial to lateral. In an embodiment, the therapeutically effective distance is a remote location that enables activation of target fibers in the at least one occipital nerve while avoiding non-target fiber activation in the at least one occipital nerve. In an embodiment, the therapeutically effective distance is 0.5-3.0 cm.

In an embodiment, the target fibers in the at least one occipital nerve comprise large diameter sensory fiber. In an embodiment, the activation of the target fibers generates comfortable sensations in a distribution of the at least one occipital nerve. In an embodiment, the comfortable sensations are generated in the region of pain. In an embodiment, the comfortable sensations are not generated in the region of referred pain. In an embodiment, the non-target fibers in the at least one occipital nerve comprise small diameter fibers. In an embodiment, the non-target nerve fibers include small diameter fibers in cutaneous tissue layers, subcutaneous tissue layers, or muscle tissue in the vicinity of the at least one occipital nerve and the electrode. In an embodiment, the non-target fibers comprise motor fibers in one or more muscle(s) in a vicinity of the at least one occipital nerve and the electrode. In an embodiment, the muscles comprise one or more of the rotatores, multifidus, semispinalis cervicis, semispinalis capitis, splenius cervicis, splenius capitis, obliquus capitis inferior, or trapezius.

In an embodiment, the electrical stimulation device comprises an external, body-worn pulse generator. In an embodiment, the electrical stimulation device comprises an implanted pulse generator. In an embodiment, the electrical stimulation comprises a first parameter selected from a group consisting of: frequency, pulse duration, amplitude, duty cycle, pattern of stimulus pulses, polarity, a predetermined number of phases, and waveform shape. In an embodiment, the electrical stimulation comprises a second parameter selected from a group consisting of: frequency, pulse duration, amplitude, duty cycle, pattern of stimulus pulses, polarity, a predetermined number of phases, and waveform shape. In an embodiment, the first parameter is amplitude and the second parameter is pulse duration. In an embodiment, an optimal amplitude and pulse duration are selected by increasing the amplitude until discomfort is produced, then decreasing the amplitude and correspondingly increasing the pulse duration to activate target fibers innervating the region of pain without activating non-target fibers. In an embodiment, the modulation of central nervous system plasticity associated with chronic pain comprises an expansion of non-painful cortical representations and/or contraction of painful cortical representations and/or functional remapping of body regions in a somatosensory cortex.

The following description and the drawings disclose various illustrative aspects. Some improvements and novel aspects may be expressly identified, while others may be apparent from the description and drawings.

### DESCRIPTION OF THE DRAWINGS

The present teachings may be better understood by reference to the following detailed description taken in connection with the following illustrations, wherein:

FIG. 1 is a schematic anatomic view of the posterior head and neck, the bony anatomy, the peripheral nerves emanating from the cervical spinal levels, and the occipital nerves.

FIG. 2 is a schematic anatomic view of the posterior head and neck and the occipital nerves with their surrounding muscles.

FIG. 3 is a schematic of the innervation regions of the occipital nerves on the posterior head and neck.

FIG. 4 is a schematic of the innervation regions of the branches of the trigeminal nerve on the anterior of the head.

FIG. 5 is a diagram of regions of the head and neck where headache pain most commonly occurs.

FIG. 6A-D is a schematic non-limiting example of the primary and referred pain distributions of cervicogenic headache, while FIG. 6E-H is a schematic non-limiting example of the primary and referred pain distributions of occipital neuralgia.

FIGS. 7 to 9 are schematic examples of bilateral lead placement approaches with the associated electrode locations, stimulation patterns in the occipital nerve distribution, the sensations generated, and the corresponding regions of pain relief in the distribution of the targeted nerve and in referred region(s) of pain.

FIGS. 10A to 10C are views showing a percutaneous lead that can form a part of a peripheral nerve stimulation system, with the lead inside an introducer needle (FIGS. 10A and 10B) and placed remote to (e.g., 0.2-3 cm from) a nerve after the removal of the introducer needle (FIG. 10C). After placement of the lead and removal of the introducer (FIG. 10C), the lead may be connected to an external stimulator with a surface electrode as a return electrode, and an appropriate bandage placed over the site where the lead exits the skin.

FIGS. 10D to 10E are views showing percutaneous leads that can form a part of a peripheral nerve stimulation system, with leads placed between and remote to two target nerves after the removal of the introducer needle, along with an external pulse generator connected to the lead.

FIGS. 10F to 10G are views showing percutaneous leads that can form a part of a peripheral nerve stimulation system with the active site (electrode) placed proximal to the tip of the lead and the electrode placed remote to the target nerve, along with an external pulse generator connected to the lead.

FIG. 10 H is a view showing a percutaneous lead that can form part of a peripheral nerve stimulation system with two active sites (electrodes) spaced along the lead, each proximal to the distal tip of the lead and each placed remote to one of two target nerves, along with an external pulse generator connected to the lead.

FIG. 10I is a view showing two percutaneous leads that can form part of a peripheral nerve stimulation system, both placed remote to a target nerve, along with an external pulse generator connected to the leads.

FIGS. 10J to 10L are views showing one (FIGS. J-I) and two (FIG. L) percutaneous leads that can form part of a peripheral nerve stimulation system, placed remote to a target nerve, along with an internal pulse generator implanted within the subcutaneous tissue and connected to the leads.

FIGS. 11A to 11C are views of the posterior head and neck showing the bandaging of the externalized peripheral nerve stimulation leads and connectors for bilateral leads placed with exit sites below the hairline (FIG. 11A), below a clipped hairline (FIG. 11B), and within the hair (FIG. 11C).

FIGS. 12A to 12C are representative views of non-limiting examples of approaches to the placement of percutaneous stimulating leads with an introducer wherein the internal anatomy may be visualized under ultrasound imaging and the stimulating lead is placed using an introducer needle to a remote location (e.g., 0.5-1 cm) from the greater occipital nerve at the level of the C2 lamina. FIG. 12B additionally shows a preferred embodiment of a region of activation stimulating from the electrode, avoiding non-target fibers in the skin and muscle while fully activating the target fibers within the greater occipital nerve. FIG. 12C further shows how this region of activation changes with varying intensity of stimulation, not fully capturing the target fibers of the greater occipital nerve when the intensity is too low and activating all of the target fibers in the greater occipital nerve as well as non-target fibers in the skin and muscle when the intensity is too high.

FIGS. 12D to 12G are representative views of non-limiting examples of approaches to the placement of percutaneous stimulating leads wherein the internal anatomy may be visualized under ultrasound imaging and the stimulating lead is placed using an introducer needle to place the electrode, in these examples at the distal tip of the lead, at a remote location (e.g., 0.5-1 cm) from the target nerve, the greater occipital nerve in FIGS. 12D-12F and both the greater and third occipital nerves in FIG. 12G, at the level of the C2 lamina.

FIGS. 12H to 12I are representative views of non-limiting examples of approaches to the placement of percutaneous stimulating leads wherein the internal anatomy may be visualized under ultrasound imaging and the stimulating lead is placed using an introducer needle to place the electrode, in these examples proximal to the tip of the lead, at a remote location (e.g., 0.5-1 cm) from the target nerve, the third occipital nerve in FIG. 12H and both the greater and third occipital nerves in FIG. 12I, at the level of the C2 lamina.

FIGS. 12J to 12K are representative views of non-limiting examples of approaches to the placement of percutaneous stimulating leads with two electrodes wherein the internal anatomy may be visualized under ultrasound imaging and the stimulating lead is placed using an introducer needle to place the electrodes at remote locations (e.g., 0.5-1 cm) from the target nerves, the greater and third occipital nerves, at the level of the C2 lamina.

FIGS. 13A-13C are representative views showing a lateral cross-section of the neck at the lateral pillar of the spinal column and a non-limiting example of an approach to the placement of a percutaneous stimulating lead wherein the internal anatomy may be visualized under fluoroscopic imaging and the stimulating lead is placed using an introducer needle to place the electrode at a remote location (e.g., 0.5-1 cm) from the two target nerves, the greater and third occipital nerves. FIG. 13A shows the lead as placed, while the neck is in flexion, while FIGS. 13B and 13C show the lead stay in position without migration while the neck moves through a neutral and extended position.

FIGS. 14A-14B are representative views showing a lateral cross-section of the neck at the lateral pillar of the spinal column and a non-limiting example of an approach to the placement of a percutaneous stimulating lead wherein the internal anatomy may be visualized under fluoroscopic imaging and the stimulating lead is placed using an introducer needle to place the electrode at a remote location (e.g., 0.5-1 cm) from the target nerve, the greater occipital nerve. FIG. 14A shows the lead as placed, while the neck is in flexion, while FIG. 14B shows the lead stay in position without migration while the neck moves to a neutral position.

FIG. 15 is a representative view showing a lateral cross-section of the neck at the lateral pillar of the spinal column and a non-limiting example of an approach to the placement of a percutaneous open coil stimulating lead wherein the internal anatomy may be visualized under fluoroscopic imaging and the stimulating lead is placed using an introducer needle to place the electrode at a remote location (e.g., 0.5-1 cm) from the target nerve, the greater occipital nerve.

FIGS. 16A-16E are views of a coiled lead inserted into a block of tissue at a representative angle, and the block of tissue then deformed as is common in the mobile are of the neck into which the lead is placed.

FIGS. 17A-17O are representative leads that can form a part of a peripheral nerve stimulation system.

FIG. 18 is a view showing the external components of one embodiment, including an external stimulator, cabling, and bandage covering the exit sites of percutaneous stimulating leads.

FIG. 19 is a view showing the internal components of one embodiment, including an internal stimulator, cabling and/or wires, and electrodes of the stimulating leads.

FIG. 20 is a representative external view of one embodiment showing a percutaneous lead exiting from the skin and connecting through a lead connector and cabling to an external stimulator with a surface electrode as a return electrode.

FIG. 21A to 21B is a plan view of a sterile package containing a peripheral nerve stimulation system and instructions for use for the externalized system (FIG. 21A) and the fully implantable system (FIG. 21B).

### DETAILED DESCRIPTION

Reference will now be made in detail to embodiments of the present teachings, examples of which are illustrated in the accompanying drawings. It is to be understood that other embodiments may be utilized and structural and functional changes may be made without departing from the respective scope of the present teachings. Moreover, features of the various embodiments may be combined or altered without departing from the scope of the present teachings, e.g., features of each embodiment disclosed herein may be combined or replaced with features of the other embodiments disclosed herein. As such, the following description is presented by way of illustration only and should not limit in any way the various alternatives and modifications that may be made to the illustrated embodiments and still be within the spirit and scope of the present teachings.

The peripheral nervous system consists of nerve fibers and cell bodies outside the central nervous system (e.g., outside the brain and the spinal column) that conduct impulses to or away from the central nervous system. The peripheral nervous system is made up of nerves that connect the central nervous system with peripheral structures and does not include the brain and spinal cord. The nerves of the peripheral nervous system generally arise from but do not include the spinal column and exit through intervertebral foramina in the vertebral column (spine). Other peripheral nerves (e.g., the cranial nerves) may arise in whole or in part directly from the brain. The afferent, or sensory, fibers of the peripheral nervous system convey neural impulses to the central nervous system from the sense organs (e.g., the eyes) and from sensory receptors in various parts of the body (e.g., the skin, muscles, bones, ligaments, tendons, etc.). The efferent fibers (e.g., motor fibers) convey neural impulses from the central nervous system to the effector organs (e.g., muscles and glands).

The somatic nervous system (SNS) is the part of the peripheral nervous system associated with the voluntary control of body movements through the action of skeletal muscles, and with reception of external stimuli, which helps keep the body in touch with its surroundings (e.g., touch, proprioception, hearing, and sight). A somatic nerve is a nerve of the somatic nervous system. A somatic nerve is a peripheral nerve, and it is part of the peripheral nervous system. The peripheral nervous system includes all the nerves, nerve fibers, and/or neurons connected with skeletal muscles, skin and sense organs. The somatic nervous system consists of efferent nerves responsible for sending signals from the central nervous system through the peripheral nervous system and into the periphery (e.g., for muscle contraction). The somatic nervous system also consists of afferent (i.e., sensory) nerves responsible for sending signals to the central nervous system through the peripheral nervous system and from the periphery (e.g., from sensation from the skin, muscles, tendons, ligaments, bone, and from receptors throughout the body, including for example, receptors that sense touch, pressure, activation and/or contraction of muscle, movement, and/or body, body part, or limb movement).

A typical peripheral nerve arises from the spinal cord by rootlets that converge to form two nerve roots, the dorsal (predominately sensory) root and the ventral (predominately motor) root. The dorsal and ventral roots unite into a mixed nerve trunk that divides into a smaller dorsal (posterior) primary ramus and a much larger ventral (anterior) primary ramus. The posterior primary rami serve a column of muscles on either side of the vertebral column, and a narrow strip of overlying skin. All of the other muscle and skin in the periphery is supplied by the anterior primary rami.

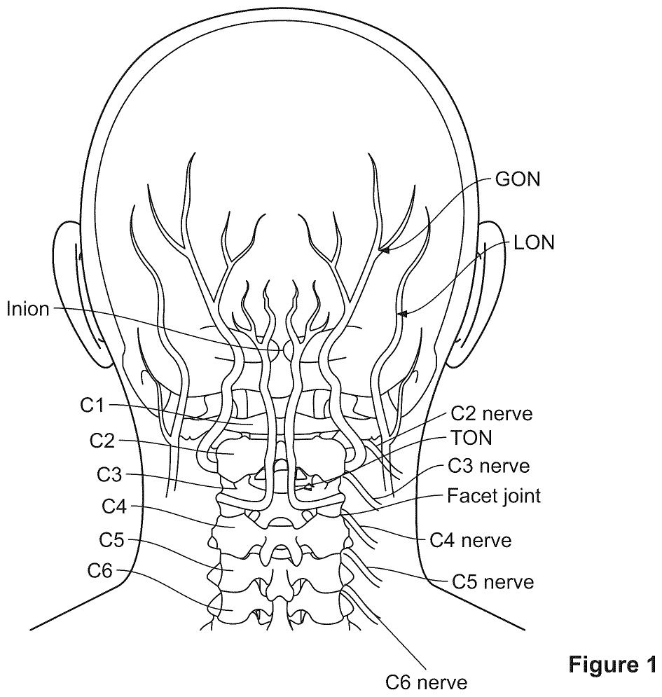

Peripheral nerves of the head and neck can be traced back (proximally toward the central nervous system) to one or more of the nerve roots in the cervical region of the spine or, in the case of cranial nerves, directly to the brain. While nerve roots (also called peripheral nerve roots or roots of the peripheral nerve(s)) may also be termed spinal nerve roots (e.g., because they can be considered to connect the peripheral nerve to the central nervous system at the level of the spine), it is to be appreciated that nerve roots are outside of (or peripheral to) the central nervous system and are part of the peripheral nervous system. The peripheral nerve roots can be generally categorized by the level of the spine where the roots exit the spinal cord, such as cervical (generally in the head/neck, designated C1 to C8), thoracic (generally in chest/upper back, designated T1 to T12), lumbar (generally in lower back, designated L1 to L5); and sacral (generally in the pelvis, designated S1 to S5). It is therefore to be appreciated that the term “cervical nerve roots”, “C# nerve root” and “C# nerve” (where the # is 1-8 and C# is the C1, C2, C3, C4, C5, C6, C7, and/or C8 nerve root or nerves) can be used to describe peripheral nerves and/or fibers of peripheral nerves that are outside of (and not part of) the central nervous system (FIG. 1).

The following descriptions are non-limiting examples of peripheral nerves of the head and neck that may be targeted or affected by the present system disclosed herein. This is not an exhaustive list, and it should be recognized that there are other peripheral nerves that are not listed that can be targeted by the present system disclosed herein.

The occipital nerves are the primary source of sensory innervation of the posterior head and neck and are frequently involved in the generation of head and neck pain from multiple different etiologies such as occipital neuralgia, cervicogenic headache, and migraine. The occipital nerves consist of the greater, lesser, and third occipital nerves on the left and right side.

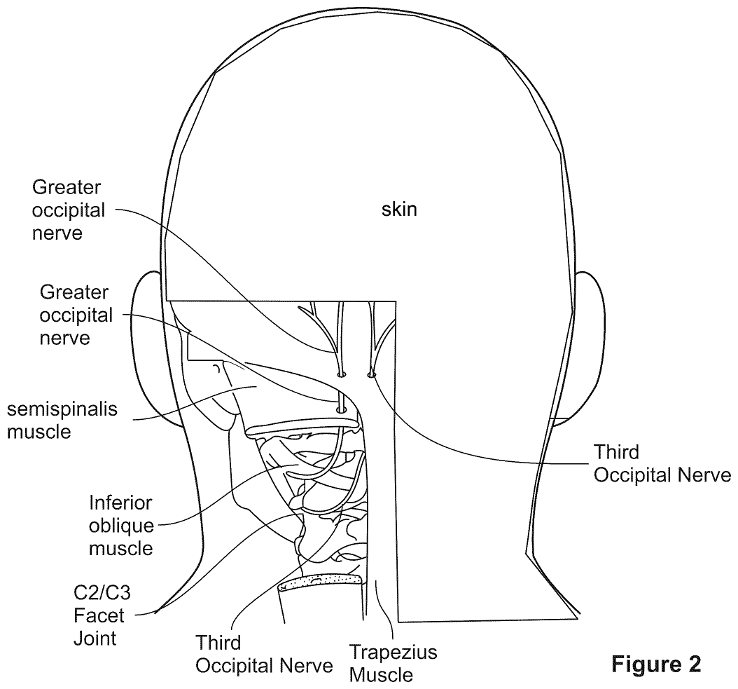

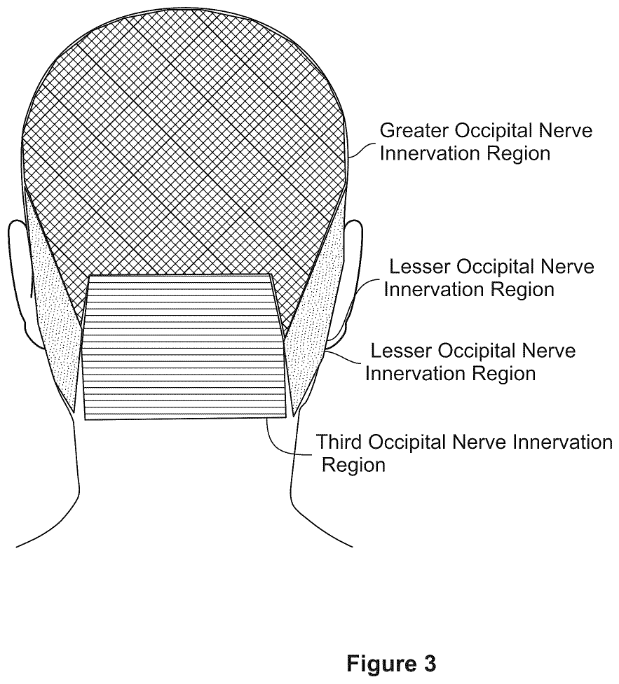

The greater occipital nerves (GON) arise from the medial branch of the dorsal, or posterior, ramus of the C2 nerve. As the GON branches off and turns dorsally, or posteriorly, after the C2 nerve exits from the intervertebral foramen, it additionally turns medially, travelling superficial to the C2 vertebra (FIGS. 1-2). GON wraps around the inferolateral border of the inferior capitis oblique muscle (or obliquus capitis inferior muscle, OCIM) such that, over the C2 lamina, the GON runs between the inferior capitis oblique and the semispinalis capitis muscles, with the prior on the medial anterior side of the nerve, or deep to the nerve. At approximately the level of the C1 vertebra, the GON begins to run in a mostly superior direction within the fascial plane between the inferior capitis oblique and the semispinalis capitis muscles. The GON pierces the semispinalis capitis then ascends superiorly in the fascial plane between the semispinalis capitis and trapezius muscles before piercing through the trapezius muscle lateral to the inion (the bony occipital protrusion of the skull). Above the level of the inion, as it continues superiorly to the vertex or peak of the skull, the GON lies subcutaneously between the cutaneous tissue and the skull, accompanied laterally by the occipital artery. The GON is a sensory nerve that innervates the skin and other tissues of the back of the head and neck (e.g., the occipital region) (FIG. 3). GON or a subset of nerve fibers from GON and/or C2 nerve may also interact with the trigeminal nerve (a cranial nerve of the peripheral nervous system responsible for innervation of the top and anterior portions of the head and face) or a subset of nerve fibers from the trigeminal nerve at the convergence of the two peripheral nerves in the trigeminal nucleus of the brainstem, which is also known as the trigeminocervical complex.

The lesser occipital nerves (LON) form from the cervical plexus, which is a network of nerve fibers that emerge from the ventral, or anterior, rami of the C1-C4 nerves (e.g., C1, C2, C3, and/or C4 nerves). LON ascends lateral and superficial to the GON and curves around the posterior border of the sternocleidomasteoid muscle, ascending along the side of the head behind the ears and innervating the skin of the posterolateral head (FIGS. 1-3).

The third occipital nerves (TON) arise from the medial branch of the dorsal, or posterior, ramus of the C3 nerve. The medial branch of the dorsal ramus of C3, which includes TON, branches off after the C3 nerve exits the intervertebral foramen, turning dorsally and medially at the level of the C2/C3 facet joint, inferior to the GON (FIGS. 1-2). Fibers of the C3 medial branch including TON cross medially at, just superior, or just inferior to the C2/C3 joint, innervating the C2/C3 joint itself. At the level of the C2 vertebra, the TON nears the midline and turns in a mostly superior direction. TON ascends just medial to the greater occipital nerve, piercing the trapezius muscle at approximately the level of C1-C2 and terminating in the lower head below the inion (e.g., the tip of the external occipital protuberance (EOP), the midline bony prominence in the occipital bone from which the ligamentum nuchae and trapezius muscle attach, which can be found, located, and/or identified with multiple means including imaging and/or palpation, which can mark the internal attachment of the tentorium cerebelli). TON innervates the C2/C3 facet joints, portions of the semispinalis capitis muscle, and supplies sensory innervation of the back of the head and neck below the inion (e.g., the suboccipital region) (FIG. 3).

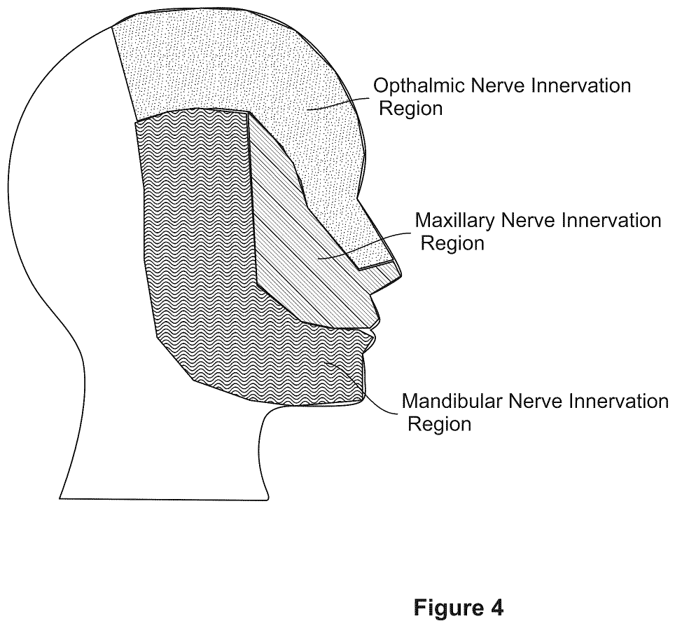

The trigeminal nerve is the fifth cranial nerve and has both sensory and motor functions. The trigeminal nerve has three major sensory branches: the ophthalmic branch, innervating the top of the head, the forehead, around the eyes, and the bridge of the nose; the maxillary branch, innervating the temples, cheeks, and upper lip; and the mandibular branch, innervating the chin, jaw, and the side of the head just anterior to the ear (FIG. 4). The trigeminal nerve arises from the brainstem, within the bony confines of the skull, and traverses through small openings in the skull to the subcutaneous tissue after branching into numerous smaller nerves.

Types of neural cells, axons, nerve fibers, or physiological structures that may be affected by the invention include, but are not limited to, functional afferent types A and C axons and efferent type A axons. The afferent axons may be classified as Aα (Type Ia or Ib), Aβ (Type II), Aδ (Type III), or C (Type IV). Afferent A fibers therefore refer to Type Ia, Ib, II, and/or III.

The term “large diameter A fibers” or “large diameter sensory fibers” or “large diameter fibers” refers to Act and/or Aβ fibers, or Type Ia, Ib, and/or II. Aα (Type Ia) fibers are associated with the primary sensory receptors of the muscle spindle, such as for transducing muscle length and speed. These fibers are myelinated, usually having a diameter from about 9 to about 22 micrometers (μm), although other diameters have been observed and may be included, and a conduction velocity of about 50 to about 120 meters per second (m/s), which is proportional to the diameter of the fiber for both this type and other types of myelinated fibers. Aα (Type Ib) fibers are associated with Golgi tendon organs, such as for sensing and transducing muscle contraction. These fibers are myelinated, having a diameter from about 9 to about 22 micrometers (μm) and a conduction velocity of about 50 to about 120 meters per second (m/s). Aβ (Type II) fibers are associated with the secondary sensory receptors of the muscle spindle, such as for sensing and transducing muscle stretch. These fibers are also associated with joint capsule mechanoreceptors (as senses and transduces joint angle) and all cutaneous mechanoreceptors. The cutaneous mechanoreceptors may include, but are not limited to, Meissner's corpuscles, Merkel's discs, Pacinian corpuscles, Ruffini corpuscles, hair-tylotrich (for sensing stroking/fluttering on the skin or hair), and the field receptor (for sensing skin stretch). The AO fibers are myelinated, usually having a diameter from about 6 to about 12 micrometers (μm), although other diameters have been observed and may be included, and a conduction velocity of about 33 to about 75 meters per second (m/s).

The term “small diameter fibers” or “small diameter sensory fibers” or “small diameter pain fibers” or “pain fibers” refers to A-delta and/or C fibers, or Type III and/or Type IV fibers. Aδ (Type III) fibers are associated with free nerve endings of touch and pressure (for sensing excess stretch or force), hair-down receptors (for sensing soft, or light, stroking), nociceptors of the neospinothalamic tract, and cold thermoreceptors. These fibers are thinly myelinated, having a diameter between or from about 1 to about 5 micrometers (μm) and a conduction velocity of about 3 to about 30 meters per second (m/s). C (type IV) fibers are associated with nociceptors of the paleospinothalamic tract, and warmth thermoreceptors. These fibers are unmyelinated, having a diameter from about 0.2 to about 1.5 micrometers (μm) and a conduction velocity of about 0.5 to about 2.0 meters per second (m/s).

As used throughout the remainder of this application, the term “occipital nerve” may include the greater, lesser and/or third occipital nerves, singly or in combination, depending upon the context of usage or unless context or this disclosure suggests otherwise. In the same manner, the term “electrode” will mean the apparatus (including a number of subcomponents, described in more detail below) that is inserted percutaneously into a patients body to deliver electrical stimulation. The term “contact” or “electrode contact” may also be used to refer to the electrode and/or one or more subcomponents of the electrode, and “electrode” can be used in some cases interchangeably with “contact” unless context or this disclosure suggests otherwise. The “lead” encompasses the electrode but also includes wires and connections to the electrical pulse generator and other aspects, components, or features, such as insulation and/or anchoring and/or fixation components. The wires of the lead that are not directly associated with the electrode are necessarily protected (e.g., by insulating overwraps) to avoid delivering unintended electrical stimulation to non-targeted tissue. The system and method also encompass return electrodes and other structure commonly associated with, and necessary for the proper operation of neurostimulation.

The system and method enable selective and/or preferential activation of target nerve fibers (e.g., Type Ia, Ib, and/or II fibers, large diameter sensory fibers, and/or A-alpha and/or A-beta fibers) in the occipital nerve(s) while avoiding unwanted muscle contractions, sensations of pain/discomfort and/or activation of non-target nerve fibers (e.g., small diameter pain fibers, A-delta fibers and/or C fibers, and/or Type III and/or Type IV fibers). The present invention is designed to overcome limitations of the prior art wherein the use of conventional leads with multiple electrodes per nerve and the resulting multiplicity of points at which the stimulation was delivered often simultaneously activated the non-target nerve-fibers and/or other non-target structures leading to unwanted muscle contractions and/or general discomfort for the patient during treatment. The present invention delivers stimulation that is more preferential and/or selective for target large diameter fibers and reducing or eliminating or avoiding the activation of non-target fibers or structures. This successful, preferential and/or selective activation is achieved in part by placing self-anchoring leads (e.g., with distal anchoring mechanism and/or with a shape permitting tissue ingrowth) within electrical proximity (e.g., 0.5-3 cm) to the occipital nerve(s) innervating the region of pain but not in direct physical contact with the occipital nerve (e.g., the electrode is placed to stimulate the target fibers selectively without physically touching the occipital nerve). Ultimately, the preferred distance to selectively activate the target fibers while avoiding activation of non-target fibers will depend upon factors such as the strength of the pulsed signals used for therapy and other stimulation parameters (e.g., pulse duration, amplitude, polarity), the waveform shape and/or pattern of pulses, electrode dimensions (e.g., geometry, length, diameter, profile, shape), the sensitivity of the nerve fibers to electrical stimulation, and the conductivity/impedance of the patient's tissue in the targeted area, and a verification step described in greater detail below will customize the parameters to the needs of each patient.

Preferentially and selectively targeting large diameter sensory fibers (e.g., A-alpha and/or A-beta fibers, and/or Type Ia, Ib, and/or II fibers) in the target peripheral nerve(s) may generate comfortable sensations or paresthesia in the region of innervation of the targeted nerve(s), while avoiding generating uncomfortable sensations (e.g., pain or discomfort such as from the activation of small diameter sensory fibers, and/or C and/or A-delta fibers, and/or Type III and/or IV fibers). The activation of the peripheral nerve(s) to generate comfortable sensations offsets, reduces, attenuates, prevents, and/or prevents the transmission of painful signals produced in or emanating from the region of pain that is also innervated by the target nerve. The activation of the peripheral nerve(s) to generate comfortable sensations may also offset, reduce, attenuate, prevent, and/or prevent the transmission of painful signals perceived to be emanating from regions outside the area(s) innervated by the target nerve (e.g., referred pain). In another embodiment, the stimulation relieves, reduces, alleviates, and/or eliminates, prevents, or resolves pain while avoiding the generation of conscious sensations (e.g., sub-threshold stimulation) by delivering stimulation at parameters (e.g., amplitudes, frequencies, pulse widths, pulse patterns, bursting patterns, etc.) that offset, reduce, attenuate, prevent, and/or prevent the transmission of painful signals while avoiding and/or preventing activity in sensory fibers that meets or exceeds the threshold of sensory perception, for example by using amplitudes or intensities of stimulation that are below the threshold for sensory perception (e.g., sub-threshold or sub-perception), high rates, doses, and/or frequencies of stimulation (e.g., up to 1499 Hz, up to 1500 Hz, 1000-10000 Hz, 2000-5000 Hz, 1000-6000 Hz), and/or burst stimulation (e.g., a pattern of stimulation alternating on and off on the order of tenths of seconds) that avoid producing activation in targeted nerve(s) that would produce and/or be perceived as a paresthesia, comfortable, uncomfortable, and/or conscious sensation.

As used herein, “comfortable sensations” generally means that pain levels, as experienced by the patient, are decreased. Thus, any standard metric for pain can be employed at selected intervals, during the treatment and/or over a period of treatments. The pain level trend over that period is indicative of comfortable sensations. Other non-quantitative feedback, again provided by a given patient, also may augment or provide a further definition of “comfortable sensations.” Still other indicators include reports of tingling, paresthesia, and the like, which may consist of “comfortable sensations.” In some cases, it may be possible to use medical instrumentation to detect and/or quantify responses within the patient's body that are suggestive of pain or a lack thereof. Of particular import is that application of the electrical stimulation will avoid creating a nerve block which causes a patient to lose feeling or motor function in the neck because blocking sensory and/or motor fibers can make functional tasks difficult (e.g., rotating the head and neck properly can be affected if sensory and/or motor fibers in the TON are blocked, numbness in the back of the head can be uncomfortable for the patient if the sensory fibers in the GON are blocked etc.). As non-limiting examples, the system provides pain relief while avoiding producing block in large diameter sensory fibers (e.g., Act and/or AP fibers, and/or Type Ia, Ib, and/or II fibers), and/or small diameter sensory fibers (e.g., A-delta fibers, Type III fibers), and/or small diameter pain fibers (e.g., C fibers, Type IV fibers), and/or motor fibers.

It is to be appreciated that the comfortable sensation(s) could be described as one or more of, including but not limited to, vibrating, buzzing, prickling, pins and needles, thumping, heat/warmth, cold/cool, trickling, pulsing/pulsating, comfortable, tightness, pressure, contraction, etc. Evoking comfortable sensations or paresthesia in the region of pain while avoiding evoking uncomfortable sensations, including, but not limited to, pain, discomfort, unwanted muscle tension, contraction, tightness, and/or motor effects of muscle tension, contraction, or tightness in response to stimulation, confirms correct placement of the lead(s) and indicates stimulus intensity is sufficient to reduce pain.

The present invention is designed to address pain in the head and neck, especially in the region of the head and neck innervated by the occipital nerves and also including pain such as referred pain in other regions outside of the direct innervation area of the occipital nerves. The pain which the present invention is designed to address can be caused by a variety of injuries or conditions, such as occipital neuralgia, cervicogenic headache, occipital migraine, post-traumatic headache such as following traumatic brain injury (TBI), injury to certain structures in the neck or cervical spinal region, and other types of headache. As one non-limiting example of the magnitude of pain management problems associated with the occipital nerve, headache following traumatic brain injury (TBI) and other traumatic head injuries is a common and severe problem and approximately 1.4-3.8 million individuals in the U.S. general population suffer TBI or traumatic head injury each year, primarily from motor vehicle accidents, falls, and violence.

By addressing disabling pain, it is contemplated that the present invention will also help address multiple areas of psychological health, including depression symptoms, anxiety, sleep, and cognitive efficiency, which may be associated, impacted, or caused by pain in the head and neck. As a non-limiting example, the present invention may address pain such that patients experience decrease, relief from, reduction in, resolution, and/or remission of one or more psychological health issues (e.g., anxiety, depression, memory/concentration), sensory dysfunction (e.g., blurred vision, light sensitivity, tinnitus, problems with taste/smell), and physical symptoms (e.g., nausea and vomiting, insomnia, fatigue, loss of balance, dizziness). Because these difficulties can lead to significant disability, impair function, reduce quality of life (QOL), and are commonly associated with depression and post-traumatic stress disorder (PTSD), the present invention will address pain to enable improvement in function, daily activities, and quality of life.

The present invention is designed to overcome limitations of existing treatments for head and neck pain, which often have limited efficacy and/or carry risks of complications, dependence, debilitating side effects, and often provide insufficient pain relief. As a non-limiting example, the present invention is designed to provide long-term relief, management, and/or treatment for headache and decrease, reduce, and/or avoid the need for one or more pain or other headache medications whose use, overuse, and/or withdrawal is commonly associated with worsened headache pain. Additionally, the present invention is designed to provide pain relief without the use of narcotic and/or non-narcotic pain medications, avoiding the deleterious and/or disabling side effects of chronic pain management medications such as non-opioid analgesics (e.g., acetaminophen, non-steroidal anti-inflammatory drugs [NSAIDs]), ergotamines, triptans, prophylactic medications (antidepressants, anticonvulsants, beta-blockers and calcium channel blockers), and opioids, including avoiding the risk of addition and/or dependence on one or more pain medications (e.g., opioids). As another non-limiting example, the present invention is designed to be minimally invasive and provide sustained relief, reducing the need for recurring, repeated, cyclical, or scheduled treatments such as anesthetic nerve blocks, botulinum toxin (onabotulinumtoxinA) injections, and radiofrequency therapies (e.g., radiofrequency ablation, pulsed radiofrequency treatment), while also eliminating the substantial risk of complications, side effects, and irreversible nature of surgical and/or neuroablative (e.g., neurolysis, neurectomy, rhizotomy, etc.) procedures.

Many previous approaches to occipital nerve stimulation employed leads designed for use in spinal cord stimulation (e.g., designed to be placed in the epidural space where there is less tissue movement, contraction, expansion, flexion, or extension compared to the relatively mobile areas of the periphery where target nerves like the occipital nerves are found), and these leads were typically placed in subcutaneous tissue over and often in direct contact with the occipital nerves in the highly mobile neck joint where, due to the relatively large diameter and rigidity of the leads, the leads frequently migrated and/or fractured (21-66% of patients) over the course of therapy, resulting in loss of efficacy and/or requiring additional invasive surgery to remove, reposition, or replace the leads. While other systems and methods have been developed specifically for stimulation of nerves in the periphery, these approaches are still limited for occipital nerve stimulation due to the use of leads whose size, rigidity, and lack of flexibility lead to high rates of complication such as lead migration, fracture, and/or skin erosion. The present invention is designed to overcome limitations of the prior art such as prior occipital nerve stimulation regimes that used systems and methods not designed or intended for use in mobile anatomical locations such as the head and neck, and the present system and method reduce rates of lead migration, lead dislodgement, complications and surgical revision through the use of fine-wire, migration-resistant, self-anchoring, flexible leads intended to be placed in mobile areas of the periphery like the head and neck. In an embodiment, the system and method overcome many of the limitations of conventional neurostimulation systems, reducing invasiveness by enabling the placement of leads percutaneously (e.g., with or without imaging guidance, such as ultrasound or fluoroscopic guidance) and reducing infection rates and the loss of therapeutic effect due to lead migration by employing a lead that can be placed remote from the target nerve (e.g., 5-30 mm distant from the target nerve, and in some embodiments: 1-50 mm, 1-40 mm, 1-30 mm, 1-20 mm, 1-10 mm, 1-5 mm, 2-50 mm, 2-40 mm, 2-30 mm, 2-20 mm, 2-10 mm, 2-5 mm, 3-50 mm, 3-40 mm, 3-30 mm, 3-20 mm, 3-10 mm, 3-5 mm, 4-50 mm, 4-40 mm, 4-30 mm, 4-20 mm, 4-10 mm, 4-5 mm, 5-50 mm, 5-40 mm, 5-30 mm, 5-20 mm, 5-10 mm, etc. distant from the target nerve) and achieving the benefits of remote placement such as less sensitivity to movement or migration of the lead (e.g., such that stimulation can remain effective or successful even if lead movement or migration occurs) and increased selectivity and/or preferential activation of target nerve fibers while avoiding activation of non-target nerve fibers. In a preferred approach, the stimulating electrode is placed 0.5-3 cm distant from the nerve.

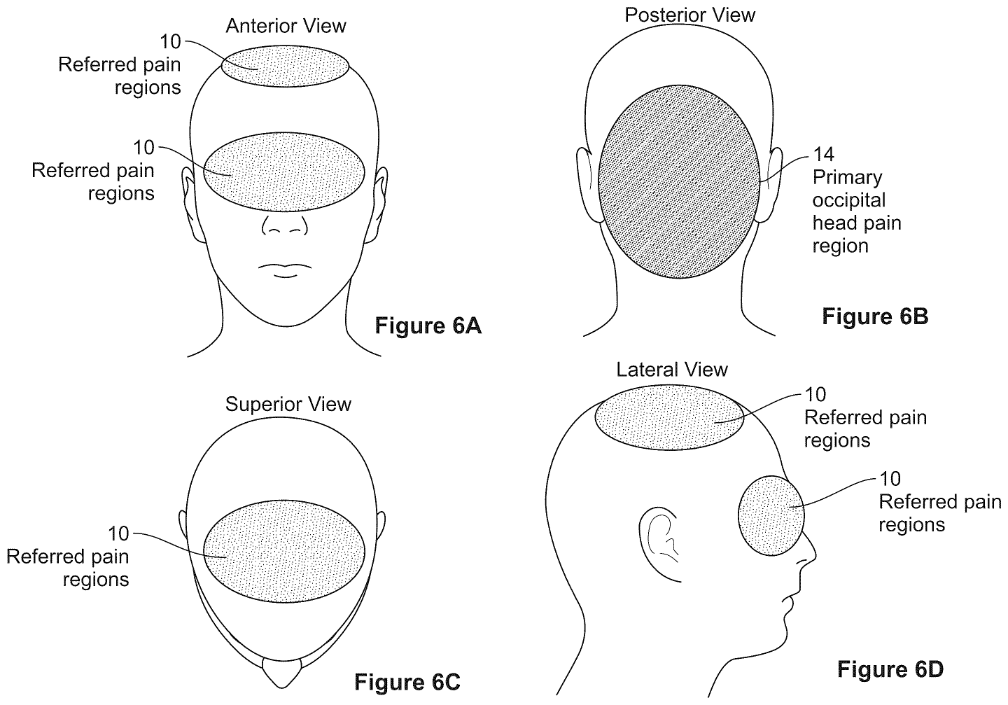

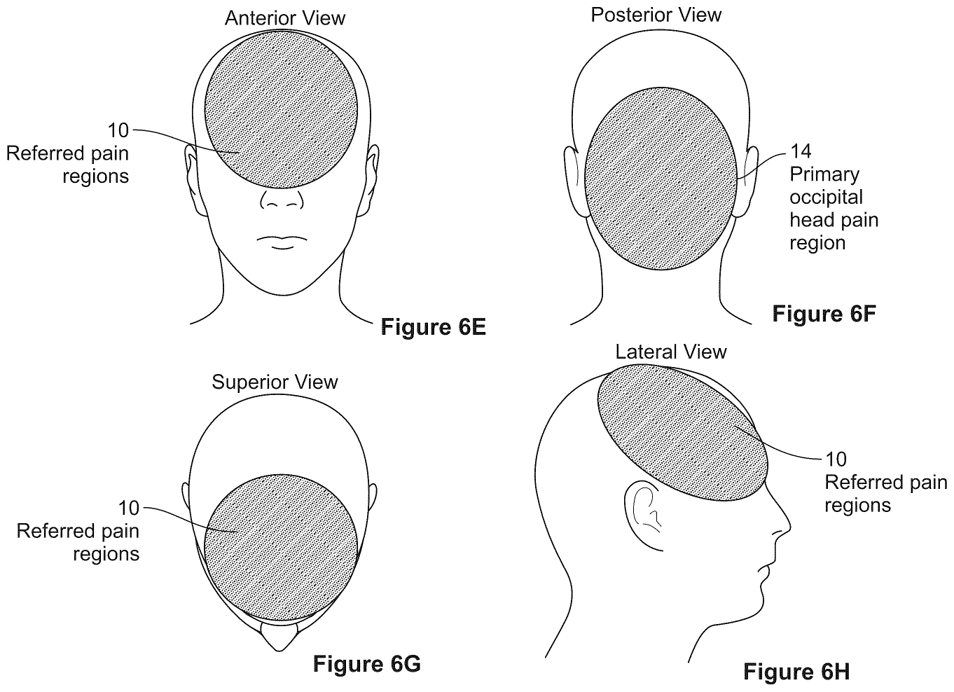

In an embodiment, the present system and method enable pain relief in areas that are in whole or in part outside the distribution and/or innervation region(s) (e.g., the tissues including but not limited to skin, connective tissue, fat, muscle, bone, nervous tissue, etc., innervated by nerve fibers from a specific peripheral nerve and/or a branch, fascicle, group of fascicles, fiber, and/or group of fibers from a specific peripheral nerve, such as the greater occipital nerve, lesser occipital nerve, or third occipital nerve) of the one or more target nerves through the stimulation of the one or more target nerve(s) to produce comfortable sensations in the distribution and/or innervation region(s) of the one or more target nerve(s) wherein the pain is not in the distribution and/or innervation region(s) of the target nerve(s) and is instead a referred pain occurring in one body region but caused by an injury, lesion, disease, trauma, and/or other source of pain located in a different body region (FIGS. 6-9). In this embodiment, stimulation of the one or more target nerve(s) (e.g., the occipital nerve(s)) may or may not produce comfortable sensations, uncomfortable sensations, and/or any other sensations in the area of referred pain that is in whole or in part outside the distribution and/or innervation region(s) of the one or more target nerve(s) (e.g., the pain is outside the distribution and/or innervation region(s) of the occipital nerve(s)) but otherwise produces a reduction in pain and/or pain relief in the area of referred pain. In one embodiment, the reduction in pain in the area of referred pain occurs as a result of inhibitory mechanisms occurring at a point of convergence of the target nerve(s) and the nerve(s) innervating the area of referred pain such that the activation of target fibers in the target nerves inhibits, prevents, mitigates, attenuates, and/or reduces the transmission, processing, perception, and/or sensation of painful signals from the area of referred pain.

In an example, stimulation of the occipital nerves can reduce pain in areas that are not directly innervated by the occipital nerves. In an example, the occipital nerves are responsible for innervation of the upper back of the neck and back of the head, but occipital nerve stimulation can help with pain in those regions as well as potential areas of pain in the front or top of the head, side of the head, behind the eyes, etc.

In an embodiment and as shown in FIGS. 6A-H, for example, referred pain may be generally understood as pain that is present and felt in a first part of the body where the cause of the pain is located in a second and different part of the body. As described, the cause of the pain may be due to any number of circumstances, including but not limited to, injury, lesion, disease, trauma, or other underlying conditions such as an imbalance in chemical activity in the body, constriction of nerves or blood vessels, osteoarthritis, compression of the nerves or nerve roots from degenerative cervical spine changes, cervical disc disease, tumors, gout, diabetes, blood vessel inflammation or vasculitis, infection, etc. Referred pain may be caused by transmission of painful signals produced in or emanating from regions outside the area(s) innervated by the target nerve. FIGS. 6A-H, for example show examples of the region where primary occipital head pain can occur and also regions of referred pain that can result from or occur in addition to and/or instead of the primary occipital head pain. FIGS. 6A-D and 6E-H, in an embodiment, show examples of referred pain regions shows as 10 and examples of a primary occipital head pain region shown as 14.

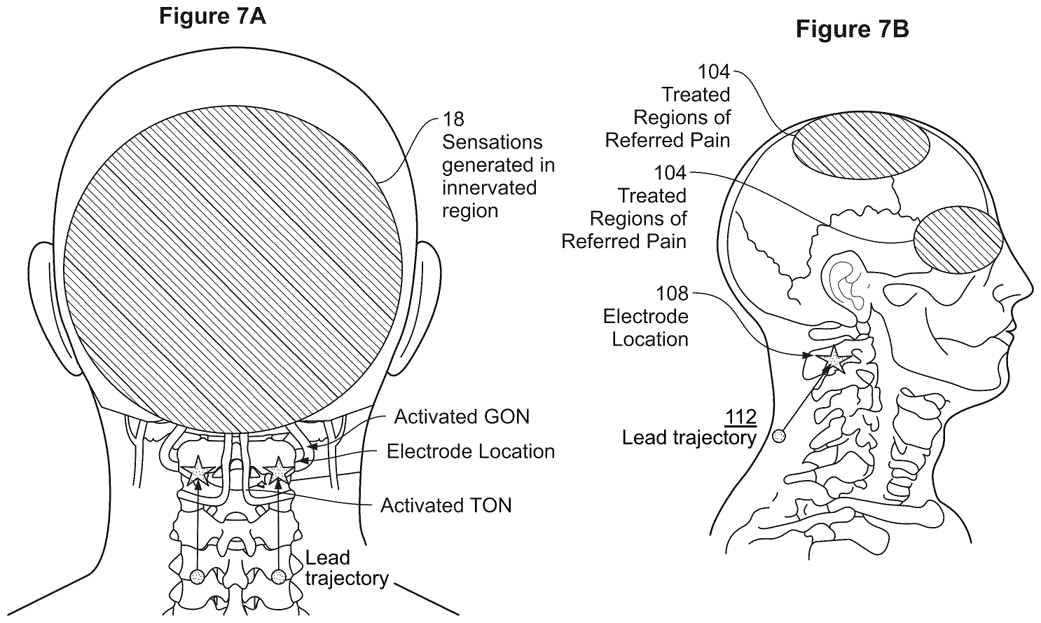

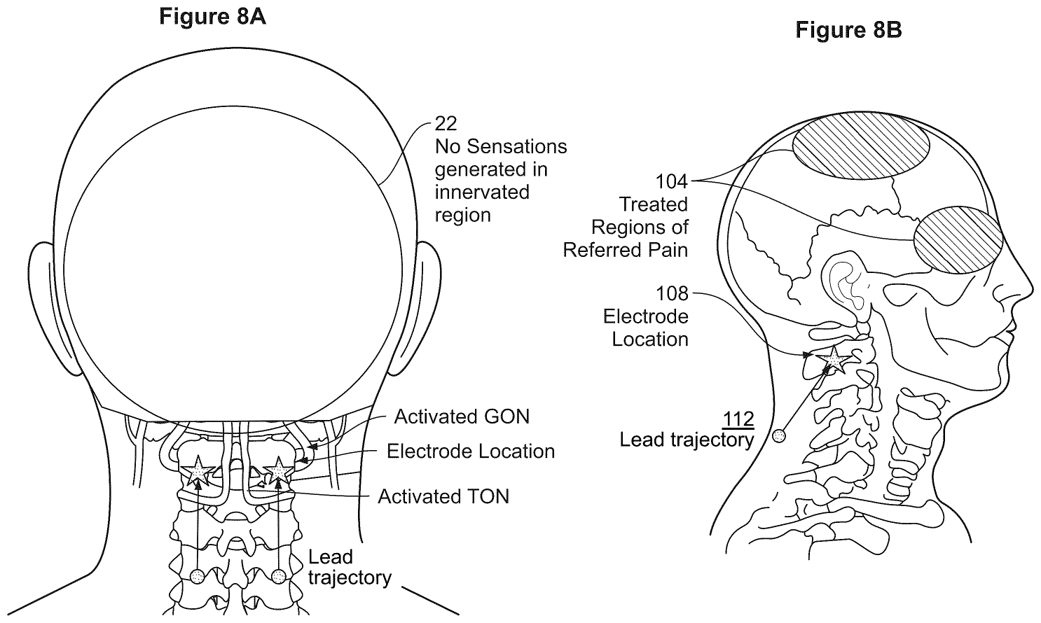

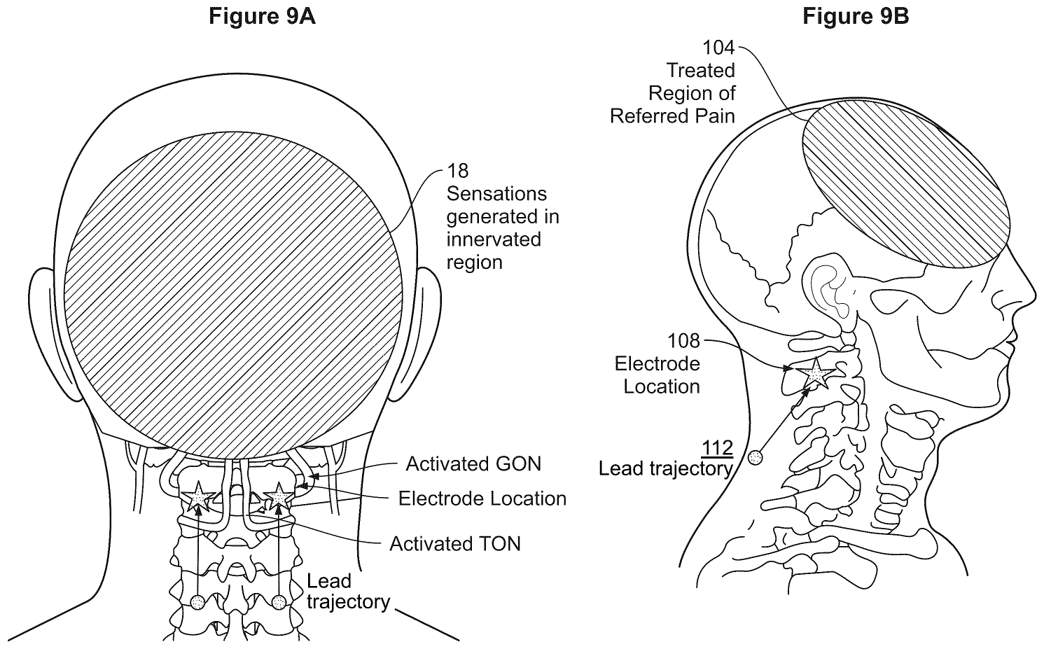

FIGS. 7A-B show regions (e.g., 18) where sensations may be generated in the innervated regions in accordance with this disclosure and regions (e.g., 104) where treatment of referred pain may result from the described lead trajectory 112 and electrode location 108 for stimulation. FIGS. 8A-B show regions (e.g., 22) where no sensations may be generated in the innervated regions in accordance with this disclosure and regions (e.g., 104) where treatment of referred pain may result from the described lead trajectory 112 and electrode location 108 for stimulation. In both instances of FIGS. 7-8, electrical stimulation may be applied to the area of the target nerve (e.g., the occipital nerve) and may provide treatment and a reduction in pain in the referred pain regions. FIGS. 9A-B show regions (e.g., 18) where sensations may be generated in the innervated regions in accordance with this disclosure and regions (e.g., 104) where treatment of referred pain may result from the described lead trajectory 112 and electrode location 108 for stimulation. As with examples in FIGS. 7-8, electrical stimulation may be applied to the area of the target nerve (e.g., the occipital nerve) and may provide treatment and a reduction in pain in the referred pain regions. As shown in FIG. 9B, the treated region of referred pain may be larger or different than that shown in FIG. 7B.

It is to be appreciated that stimulation of a target nerve that innervates the region of pain is not treatment of a referred pain even if stimulation is applied to the target nerve proximal to, outside of, or distant from the region of pain, because the target nerve still innervates the region of pain. As a non-limiting example, stimulation of a nerve to relieve pain innervated by fibers from the targeted nerve at a more distal location on the nerve, limb, appendage, extremity, or body region is not considered treatment of referred pain. As another non-limiting example, stimulation of a nerve to treat a region of phantom pain (e.g., pain perceived to be in a region of the body that has been removed and/or amputated) wherein the targeted nerve innervated the painful region and/or the perceived painful region prior to the amputation, prior to an injury, and/or in an otherwise normal, healthy body, is not considered treatment of referred pain. For example, stimulation of the sciatic nerve to treat a region of phantom pain in an amputated foot is not considered treatment of a referred pain because the sciatic nerve innervates the foot in a normal, healthy body and/or innervated the foot prior to the amputation. In contrast to these non-limiting examples, referred pain is considered a pain outside of the innervation region of a targeted nerve and treatment of the referred pain in the present invention occurs without targeting the nerve that innervates the region of pain.

In another embodiment, the reduction in pain in the area of referred pain occurs as a result of providing sensory feedback to the CNS via activation of peripheral nerve fibers in the one or more target nerve(s) to promote beneficial functional plasticity to correct imbalances in somatosensory processing and relieve chronic pain and/or centrally-maintained pain or central sensitization, thereby producing pain relief in regions, such as areas of referred pain, outside the innervation region of the one or more target nerves but whose sensory signals and/or processes converge, meet, combine with, and/or are processed in the same central nervous system regions, structures, loci, nuclei, etc., as the sensory signals and/or processes from the regions that are innervated by the one or more target nerves. Chronic pain has been suggested to involve maladaptive supraspinal structural and functional plasticity, including shifts in cortical sensory representations that correlate with the severity of pain and are caused by an imbalance of sensory information (e.g., an increase in pain information and/or a decrease in non-pain information compared to a healthy, normal baseline), and maladaptive plasticity in central neural structures such as the spinal cord and/or brain can contribute to referred pain because of the convergence of sensory input from multiple areas of the periphery in central neural structures.

In this embodiment, the present invention reconditions, reverses, mitigates, and/or treats maladaptive plasticity in central neural structures by selectively activating a sufficient number or majority of large diameter fibers in the target nerve (e.g., such as >50%, >70%, >80%, or preferably up to 100% of large diameter fibers), reversing aberrant plasticity in the cortex by shifting the balance of sensory information towards a greater amount of non-painful information and/or less painful information and thereby reducing pain in the region innervated by the target nerve(s) and/or the one or more areas of referred pain whose sensory inputs converge centrally in the same areas, regions, structures, loci, nuclei, etc., as the target nerve(s).

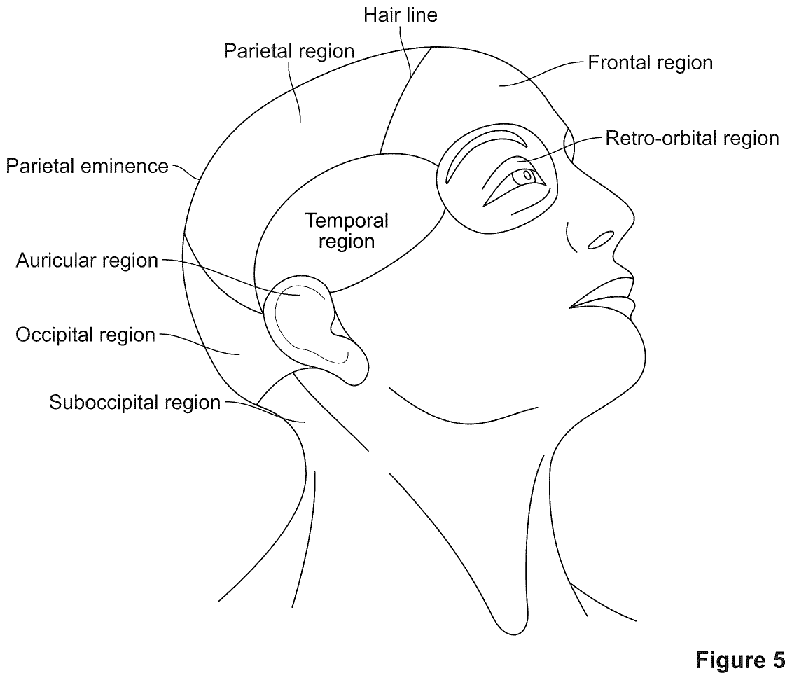

In a preferred embodiment, the present system and method produce comfortable sensations in one or more of the occipital region (i.e., the region innervated by the greater occipital nerves), and/or the suboccipital region (e.g., the region innervated by the third occipital nerves), and/or the lateral occipital region (e.g., the region innervated by the lesser occipital nerves), and/or regions secondarily affected by activation of the occipital nerves through interactions in the trigeminocervical complex or other interactions, cross-over, intercommunication, direct or indirect effects between one or more peripheral nerves, such as the region innervated by the trigeminal nerves. In another embodiment, the present system and method relieve pain while avoiding producing any sensations (e.g., without producing comfortable sensations) in one or more of the occipital region (e.g., the region innervated by the greater occipital nerves), and/or the suboccipital region (e.g., the region innervated by the third occipital nerves), and/or the lateral occipital region (e.g., the region innervated by the lesser occipital nerves), and/or regions secondarily affected by activation of the occipital nerves through interactions in the trigeminocervical complex or other interactions, cross-over, intercommunication, direct or indirect effects between one or more peripheral nerves, such as the region innervated by the trigeminal nerves. As a non-limiting example, the present system and method stimulate the GON and/or TON producing pain relief with and/or without the concomitant generation of comfortable sensations in the occipital and/or suboccipital regions in the posterior head (e.g., the regions that are directly innervated by the target nerves), while additionally producing pain relief in one or more regions of referred pain in the frontotemporal region, parietal region, retro-orbital region, or other region in the innervation region of the trigeminal nerve with and/or without the concomitant generation of comfortable sensations in the area of referred pain (FIGS. 5-9).

While previous approaches to neurostimulation have contemplated that stimulation of the neural target (e.g., peripheral nerve, spinal cord, dorsal root ganglion) that innervates the region of pain is necessary to achieve relief of pain, in one embodiment the present invention is designed to provide relief of referred pain and may or may not also provide relief of pain in the region innervated by the target nerve. As a non-limiting example, the present invention enables stimulation of a nerve to provide pain relief of a region not innervated (e.g., not directly or indirectly innervated) by the nerve that is stimulated, overcoming several limitations of the prior art. Headache pain often occurs in the frontotemporal region, parietal region, retro-orbital region, and/or other regions innervated by the trigeminal nerve and/or its branches such that previous systems and methods have sought to stimulate, target, activate, and/or block the trigeminal nerve and/or the branches of the trigeminal nerve (FIGS. 4-5). However, the branches of the trigeminal nerve, once they exit the skull, lie superficially in the subcutaneous tissue over the facial bones where there is often insufficient tissue to securely anchor the lead, erosion of the lead through tissue and/or the skin can occur, and/or the close proximity of off-target and/or non-targeted nerves whose activation is uncomfortable, painful, and/or causes other undesired effects (e.g., trochlear nerve, facial nerve, vestibulocochlear nerve, etc.) limits the effectiveness of stimulation for pain relief.

As an example, by stimulating the occipital nerves to produce pain relief in referred areas of pain such as pain in the innervation region of the trigeminal nerve and/or its branches and avoiding direct stimulation of the trigeminal nerve and/or its branches, the present invention overcomes the challenges of stimulating the trigeminal nerve directly (FIGS. 6-9). In another non-limiting example, headache pain may occur across the innervation patterns of many nerves and/or nerve branches and/or is non-specific head pain (e.g., is not located in or associated with one or more specific nerve innervation patterns, for example in some migraine headaches), and while previous systems and methods seeking to stimulate, target, activate, and/or block the nerve or nerves whose innervation patterns encompass the pain (or innervate the area or region of pain) have been limited in their ability to treat more widespread or non-specific pain, the present invention is designed to stimulate the occipital nerves to achieve a central effect (e.g., reconditions, reverses, mitigates, and/or treats maladaptive plasticity in a central neural target such as the spinal cord and/or brain where painful and non-painful sensory information are processed from the innervation regions of multiple nerves and/or body areas) and/or otherwise relieve pain in a widespread, non-specific, and/or specific region by modifying, attenuating, preventing, inhibiting, decreasing, or otherwise reducing the intensity of pain signals at a central location and/or point of convergence of the nerve(s) innervating the region of widespread pain or non-specific pain and/or a specific region or area of pain that is outside the area innervated (directly or indirectly) by the nerve that is stimulated (e.g., by the nerve to which electrical stimulation is applied, targeted, or delivered).

To provide pain relief through electrical stimulation of the occipital nerves while overcoming the challenges of conventional approaches, the system and method can do one or more (or all) of the following: a) selectively activate a sufficient number of targeted pain relieving fibers in the peripheral nerve, b) avoid stimulation of non-targeted fibers in the peripheral nerve and/or other off-target and/or non-target structures (e.g., muscle, nerve endings in cutaneous or subcutaneous tissue), c) avoid migration and/or fracture of the lead (e.g., caused by implantation in mobile tissue structures in the neck and head), and/or d) avoid unwanted damage or changes of both neurological (e.g., nerve) tissue and non-neurological tissue (e.g., muscle, connective, subcutaneous, cutaneous, dermal, skin, vascular, bone, and/or other tissue) such as avoiding, preventing, protecting against, and/or prohibiting the erosion of hardware components (such as the lead(s), component(s) of the lead, electrode(s), contact(s), anchor(s), tine(s), lead body(ies), stimulator(s), receiver(s), connector(s), cable(s), and/or other components of the system) through one or more anatomical tissues, such as preventing erosion through, within, or to the skin and/or other tissues. It is to be appreciated that these factors are inter-related along with the unique anatomy around the occipital nerves, meaning that these factors cannot be achieved without careful design and consideration to develop a suitable system and method.

For example, in the region of the occipital nerves, conventional approaches have placed leads at distal locations along the target nerve (e.g., superior or cephalad to the nuchal ridge, the C1 vertebra, or the external occipital crest or median nuchal line that descends from the external occipital protuberance to the foramen magnum where the attachment of the nuchal ligament occurs) because the tissues in those distal regions (e.g., overlying the cranial or skull bones and not overlying the cervical vertebrae) are more fixed and/or less mobile relative to tissues in the upper cervical spine where the more proximal portions of the nerve lie; however, those conventional approaches have suffered from tissue damage and/or erosion of the relatively stiff and/or large diameter leads through tissues, such as the skin, and conventional approaches have also been limited or caused clinical failure by causing discomfort (e.g., due to activation of nerve endings in the cutaneous or subcutaneous tissue) for multiple reasons including because there is insufficient tissue depth or distance between the cutaneous and subcutaneous layers and the cranial bone to achieve a remote placement from the nerve and/or because conventional approaches are unable to achieve selective or preferential stimulation or remote selective stimulation of the occipital nerve(s) in this or other anatomical locations.

Conventional approaches that have attempted to place leads at proximal locations on the occipital nerves (e.g., below the nuchal ridge such as at the C2 vertebral level) where there is more tissue depth to avoid the stimulating electrode(s) being in close proximity to the cutaneous and subcutaneous tissues have suffered from high rates of lead migration, dislodgement, erosion through the skin, and/or lead fracture because the mobility of tissues in these more proximal locations produce high levels of stress, strain, shear, and/or other forces on the leads. The use of conventional or previous leads located at proximal sites to stimulate the occipital nerves also requires the stimulating electrode(s) to be placed within, around, near, and/or intramuscularly relative to one or more muscles that lie in the vicinity of the occipital nerves, such as the rotatores, semispinalis cervicis, semispinalis capitis, splenius cervicis, splenius capitis, multifidus, oblique capitis inferior, trapezius, and/or sternocleidomastoid muscles. Thus, the present invention is designed to overcome all or a combination of these challenges simultaneously and/or at the same time with a comprehensive method and system.

While conventional neurostimulation systems have been limited by and struggled with multiple problems and challenges, such as tissue damage, erosion through tissues, erosion through or to the skin, high rates of unintended, uncomfortable activation of nerve endings (e.g., pain fibers) in the cutaneous and/or subcutaneous tissue due to the placement of leads in the subcutaneous tissue in close proximity to or in direct contact with these non-target pain fibers, unintended and uncomfortable activation of motor fibers in one or more muscles that lie in the vicinity of the occipital nerves, high rates of lead migration, dislodgement, lead fracture and/or other problems, the present invention is designed to overcome those limitations and avoid unintended, uncomfortable activation of nerve endings (e.g., pain fibers) by implanting the lead such that the stimulating electrode is located remote from the target nerve (e.g., in a preferred embodiment 5-30 mm distant from the target nerve, and in some embodiments: 5-15 mm, 5-10 mm, 1-50 mm, 5-50 mm, 2-30 mm, 2-5 mm, 2-10 mm, 3-10 mm, and/or 1-40 mm distant from the target nerve) to enable selective and comprehensive activation of a sufficient number of target large diameter fibers in the target nerve(s) to provide the desired effects, outcomes, and/or pain relief, while also avoiding unwanted and undesired effects, such as avoiding activation of non-target small diameter fibers in the cutaneous and subcutaneous tissue whose activation causes discomfort and/or undesired sensation and/or non-target motor fibers in the surrounding musculature.

One significant challenge of remote selective stimulation is the placement of the lead and the electrode in precisely the right location and to maintain the lead and the electrode in that position during treatment so that only the desired nerves and regions are stimulated without stimulating undesired nerves or regions. In an embodiment, the described methods and leads herein may assist in overcoming this challenge.

Furthermore, while conventional neurostimulation systems have been limited by and struggled with high rates of lead migration, dislodgement, fracture, and/or erosion through the skin, the present invention is designed to overcome those limitations through the use of lead(s) that are flexible, coiled (e.g., with an open coil and/or closed coil design), self-anchoring (e.g., can be anchored without sutures, surgery, and/or the use or deployment of additional components or hardware (e.g., in addition to what is already part of the lead)), migration resistant, fracture resistant, and/or infection resistant, wherein the coiled structure may enable the lead(s) to flex and bend when subjected to forces rather than migrate or fracture, and wherein the lead(s) may possess mechanical properties in terms of flexibility and fatigue life that provide an operating life (e.g., the duration of therapy) free of mechanical and/or electrical failure, taking into account the forces, stresses, challenges, and dynamics of the surrounding tissue in the areas around the occipital nerves (e.g., stretching, bending, pushing, pulling, crushing, etc.). Placement of the lead remote from the target nerve in the present invention also overcomes many other limitations of conventional neurostimulation systems such as the potential for nerve injury and the risk of loss of stimulation effects due to lead migration by the present invention enabling the placement of leads percutaneously (e.g., with or without imaging guidance, such as ultrasound or fluoroscopic guidance) without a surgical procedure thereby reducing invasiveness and infection rates, and also avoiding, preventing, and/or reducing the loss of therapeutic effect due to lead migration.

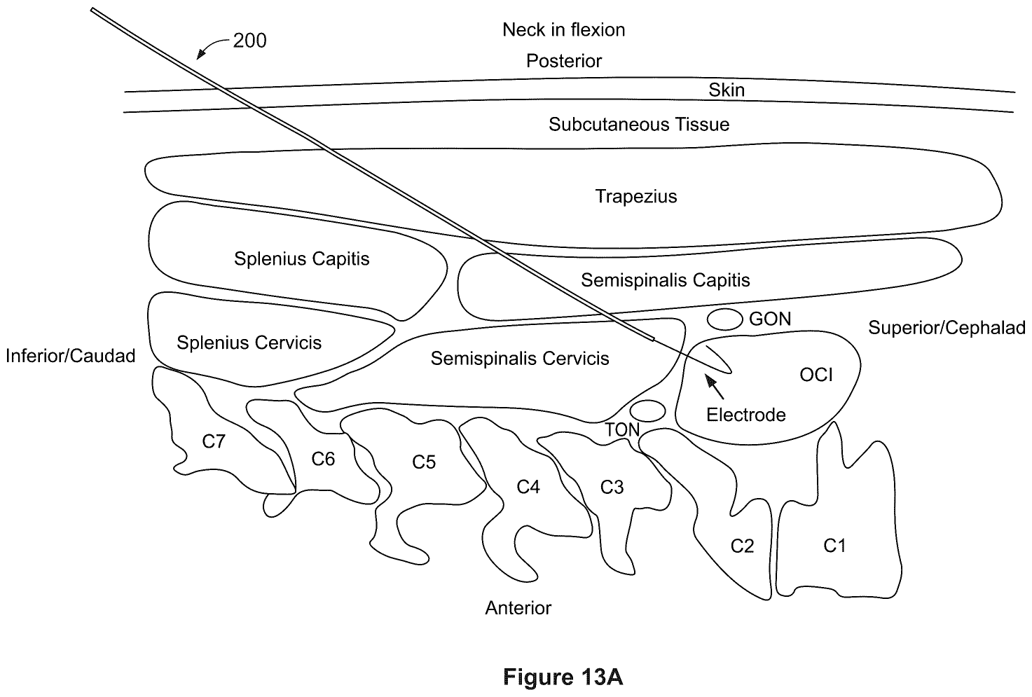

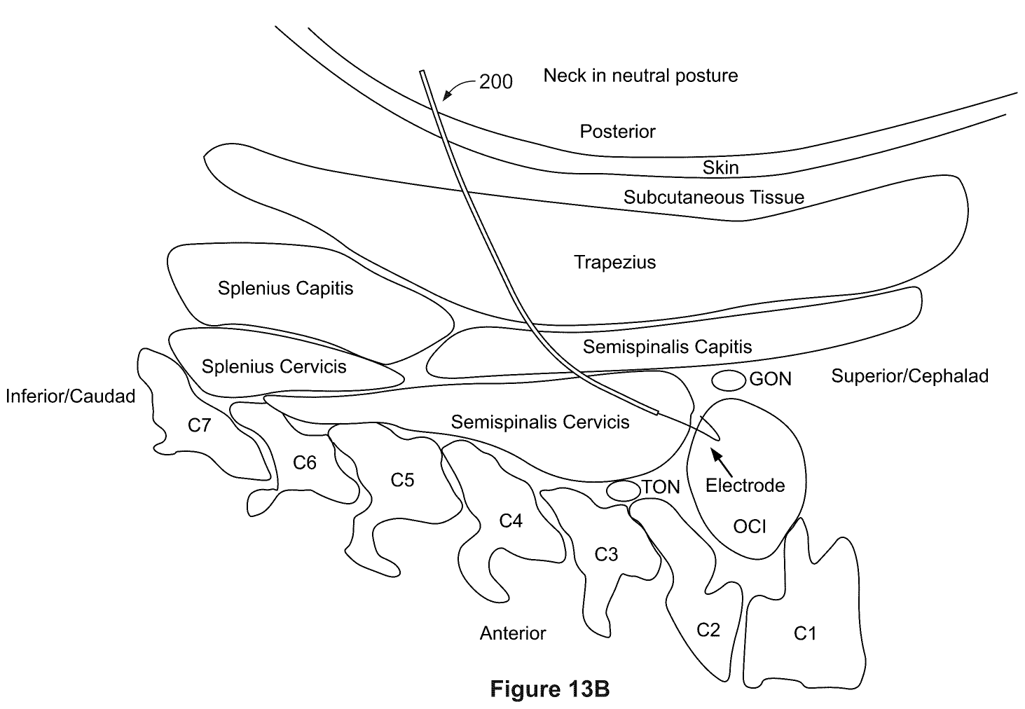

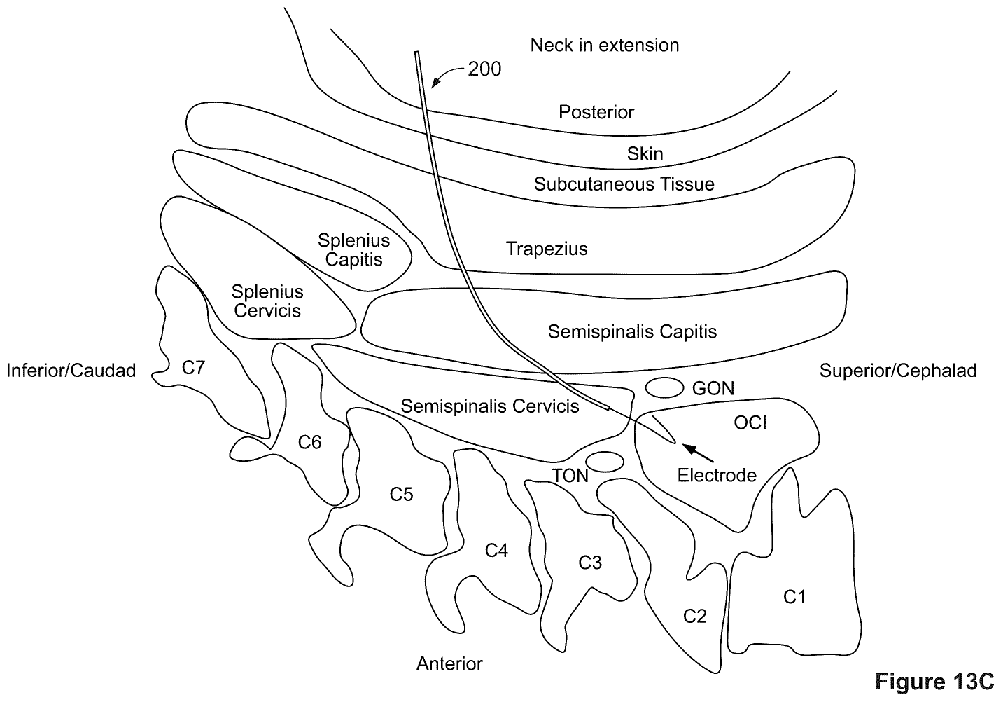

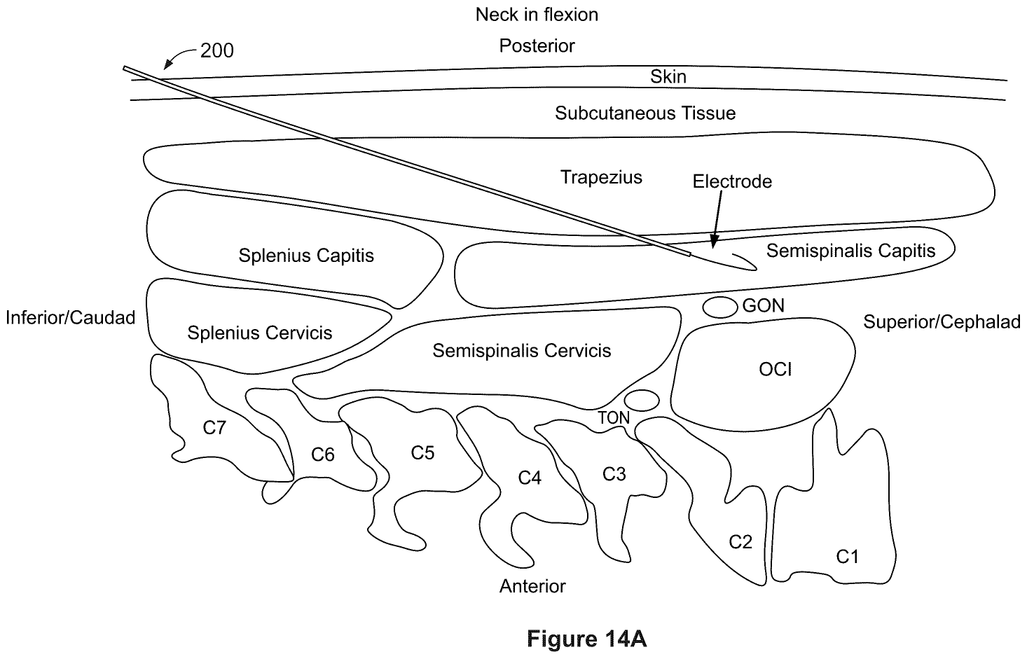

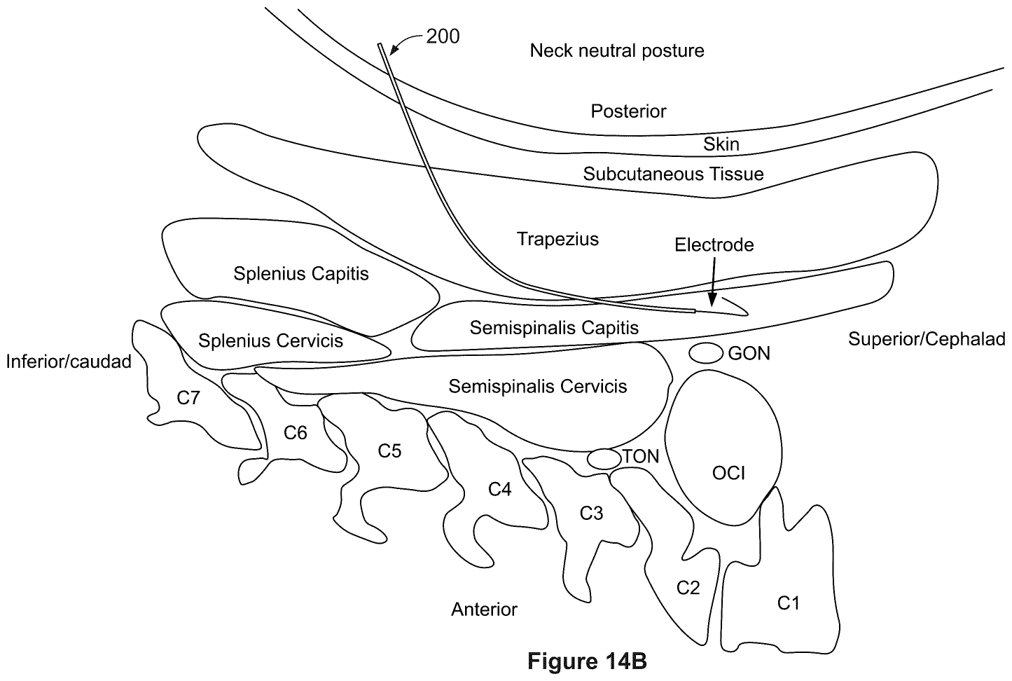

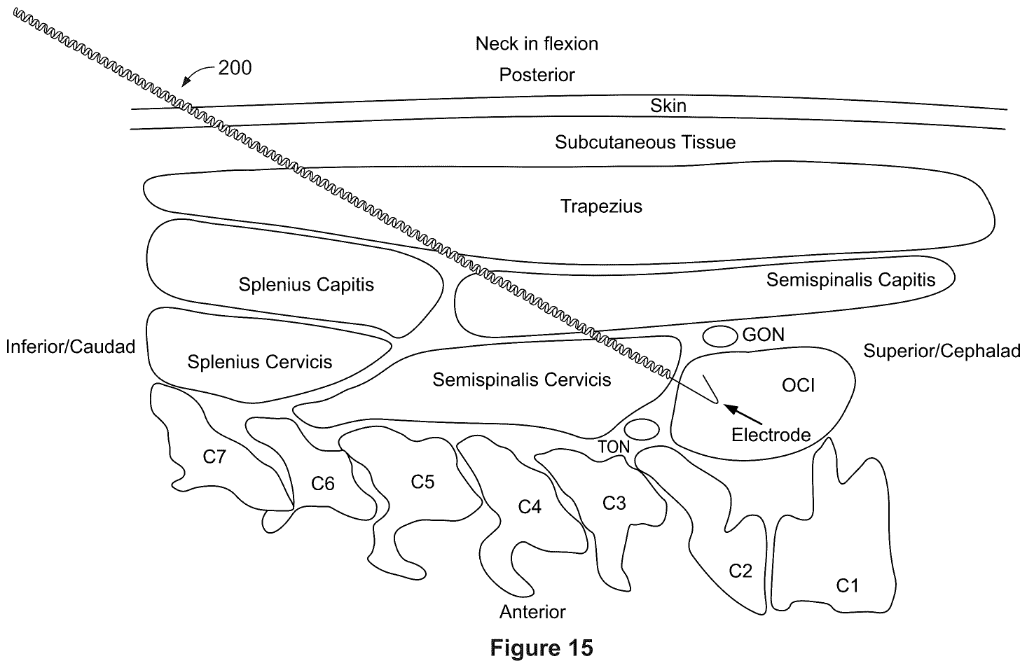

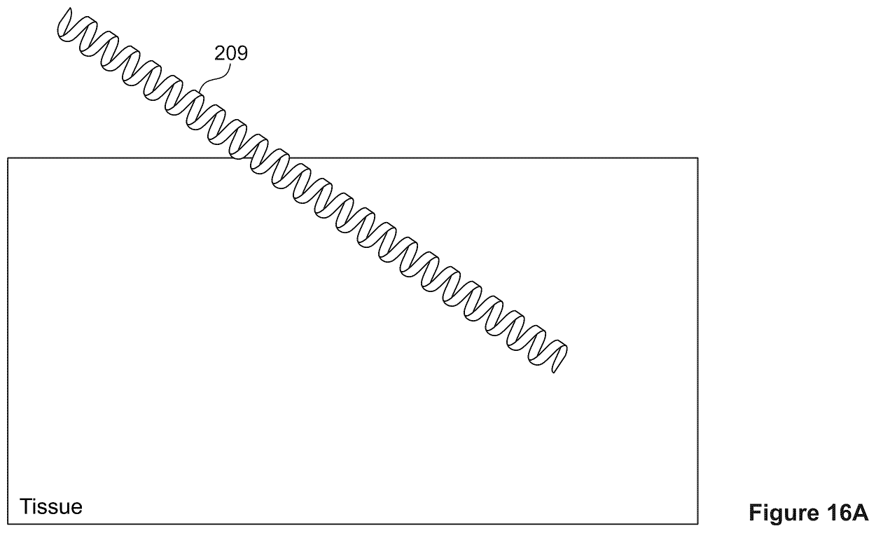

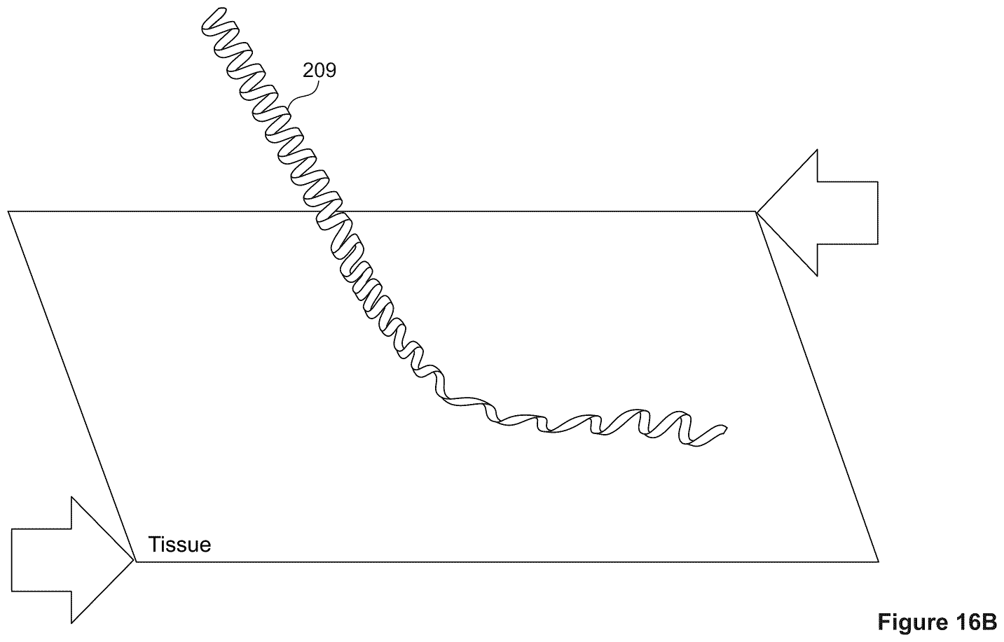

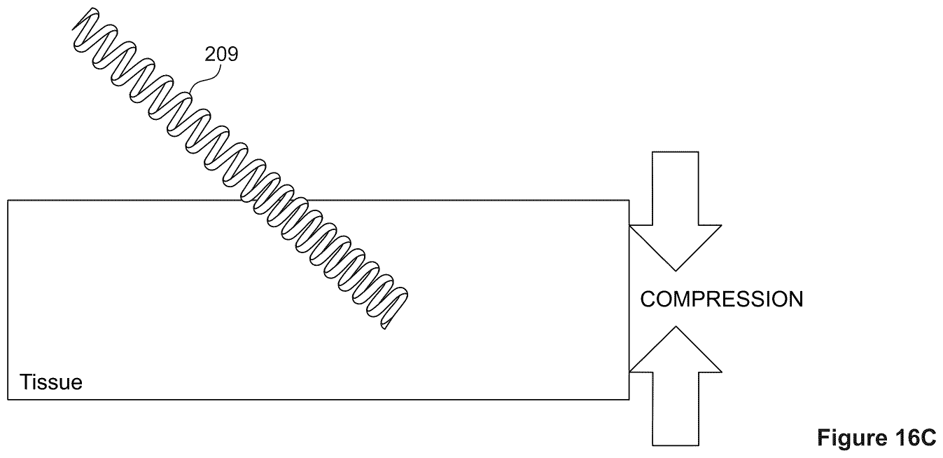

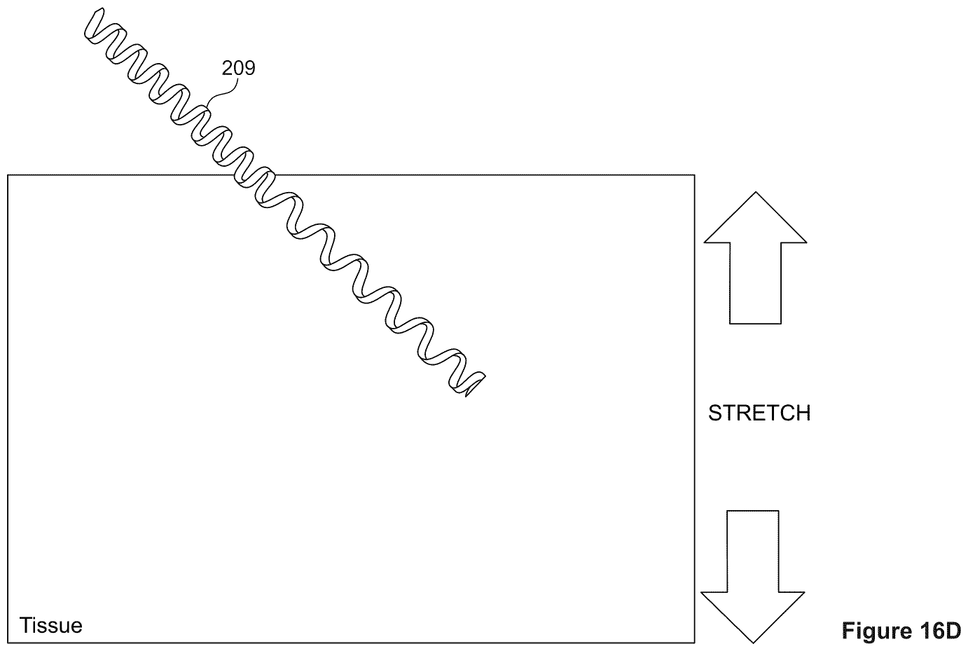

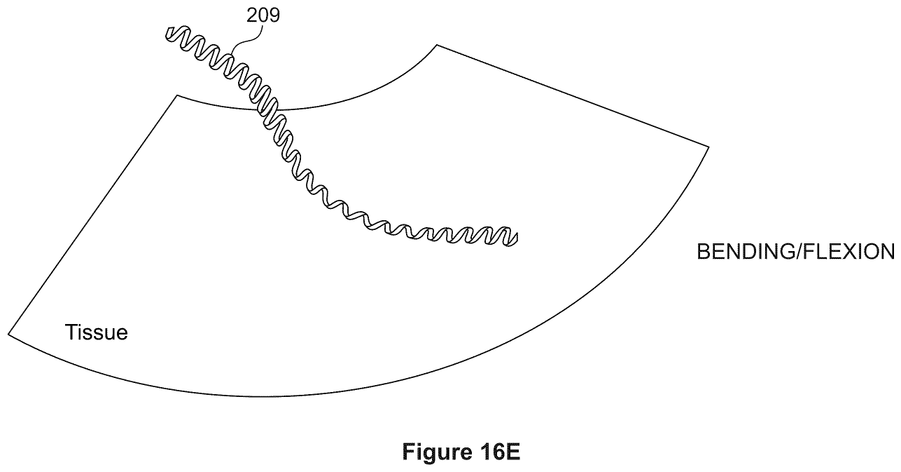

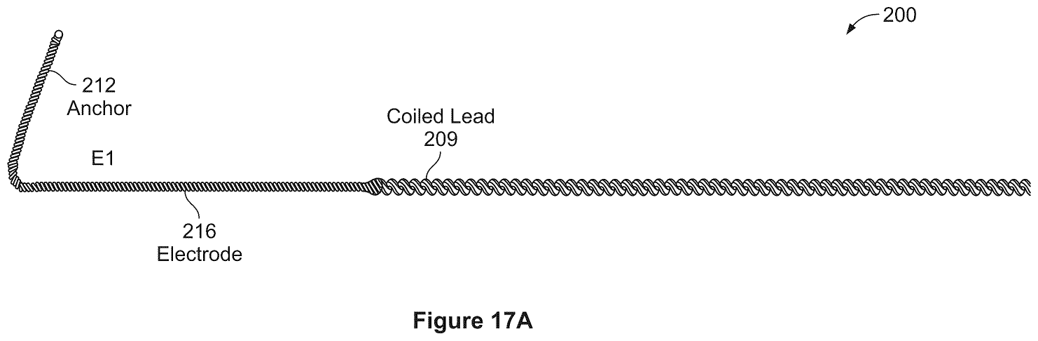

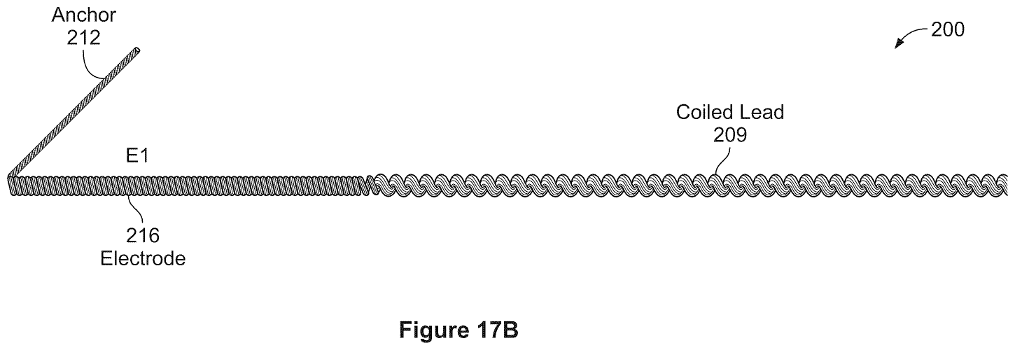

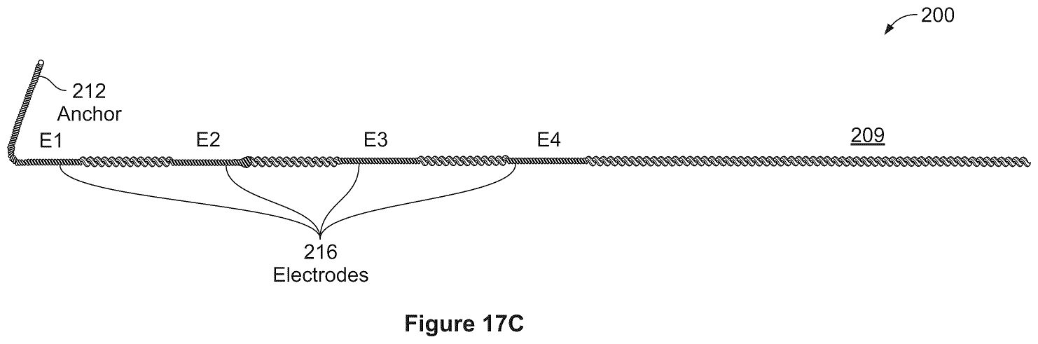

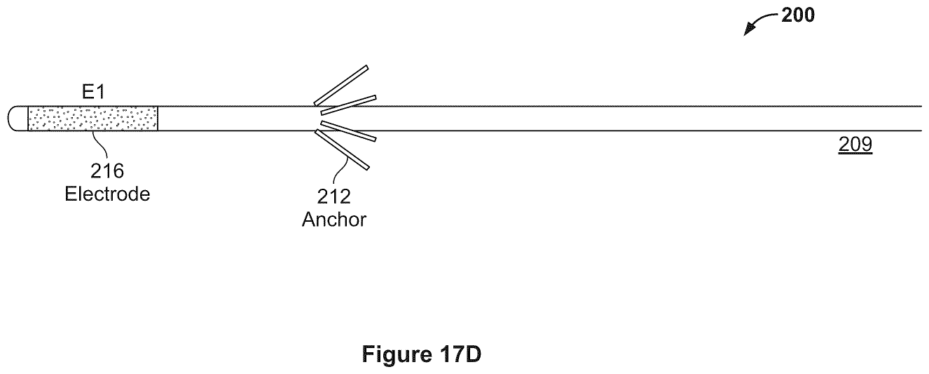

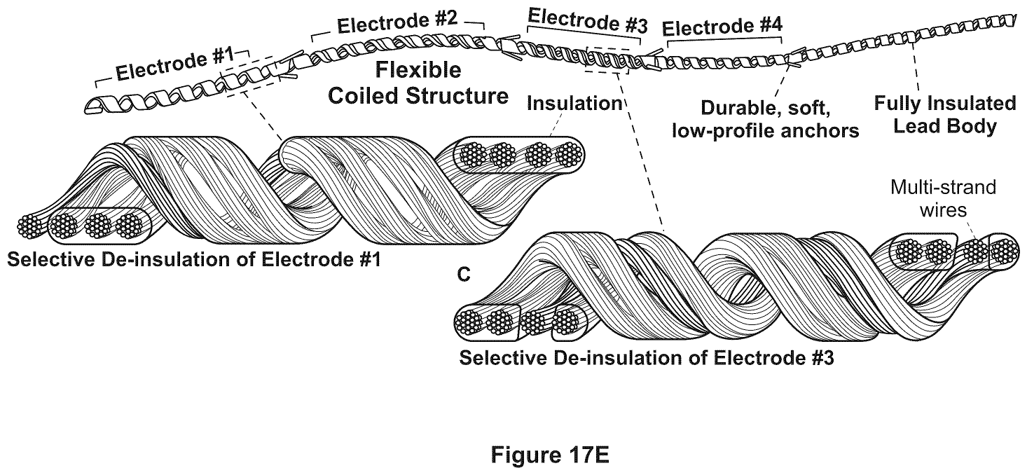

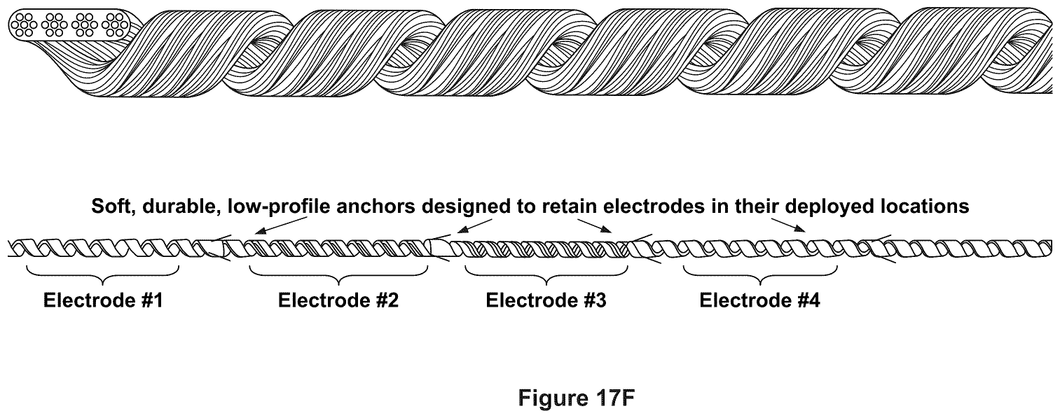

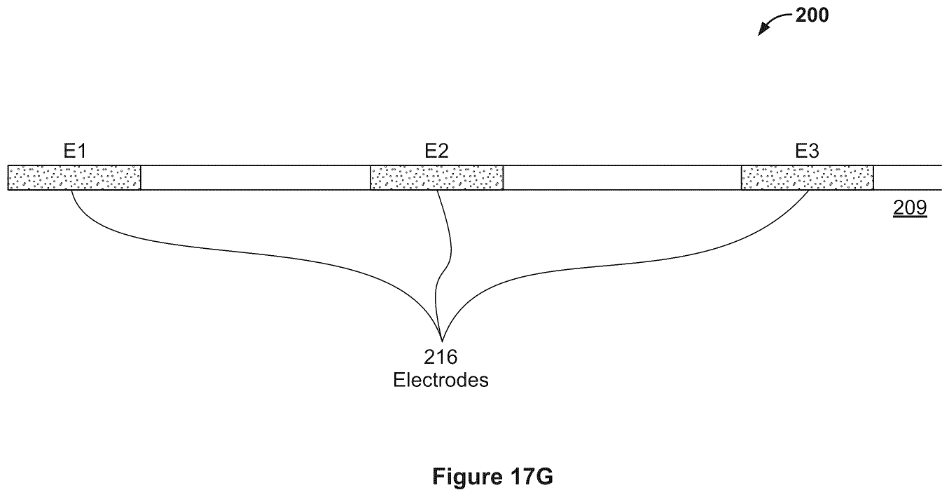

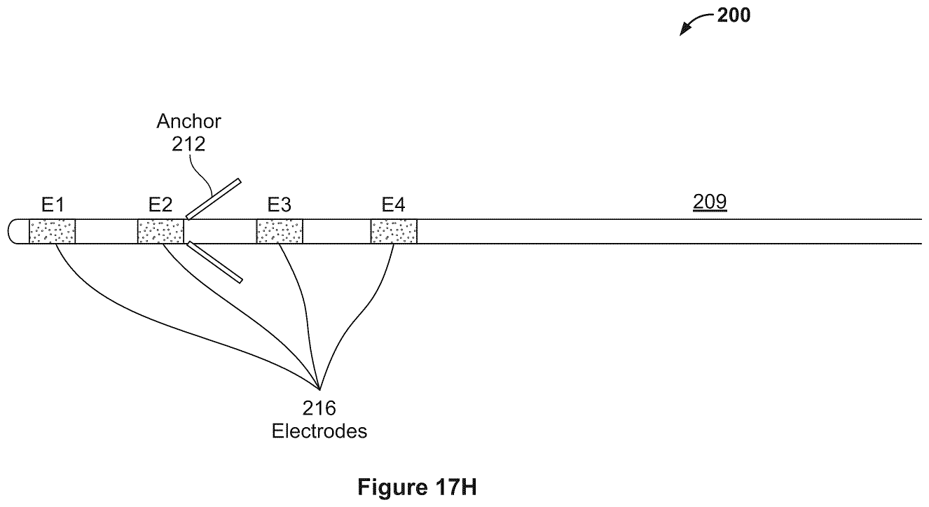

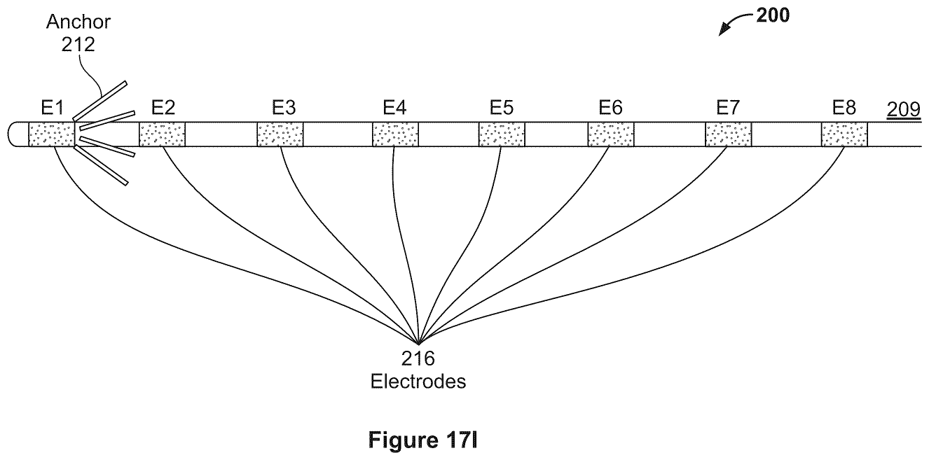

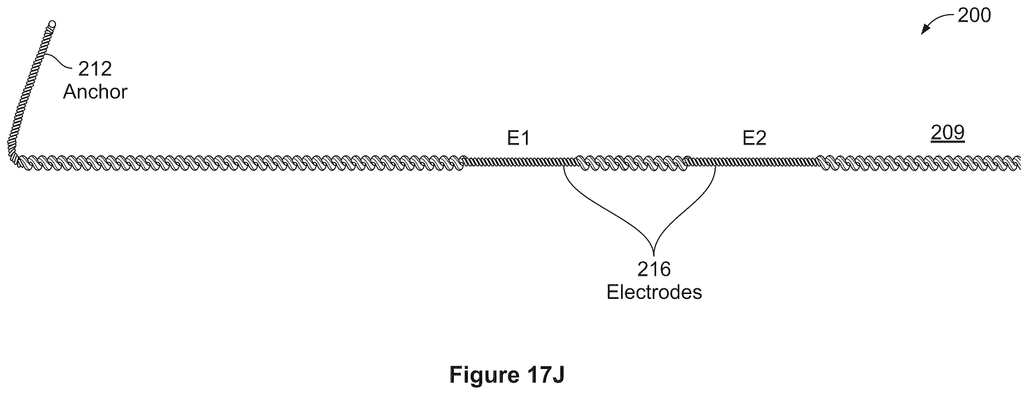

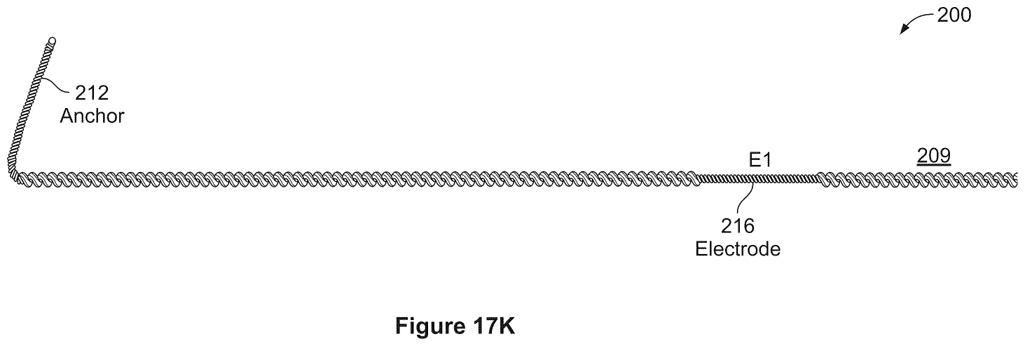

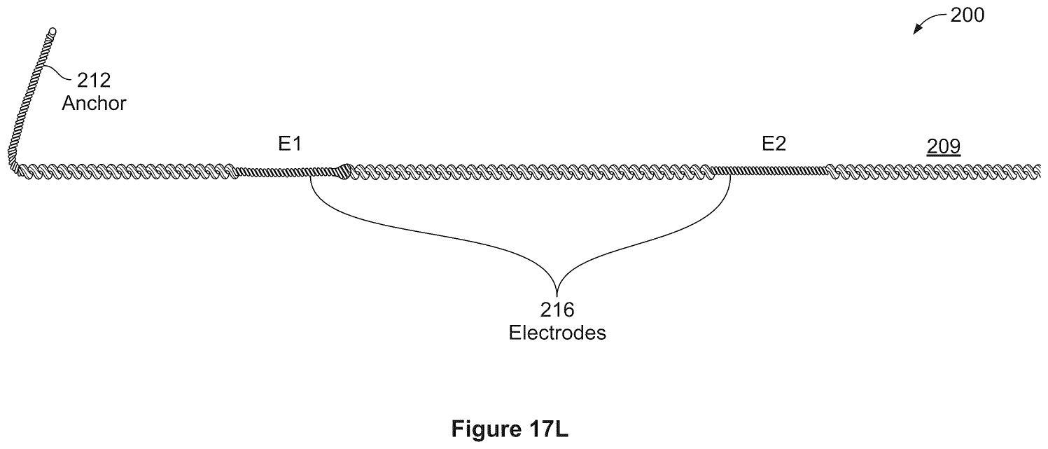

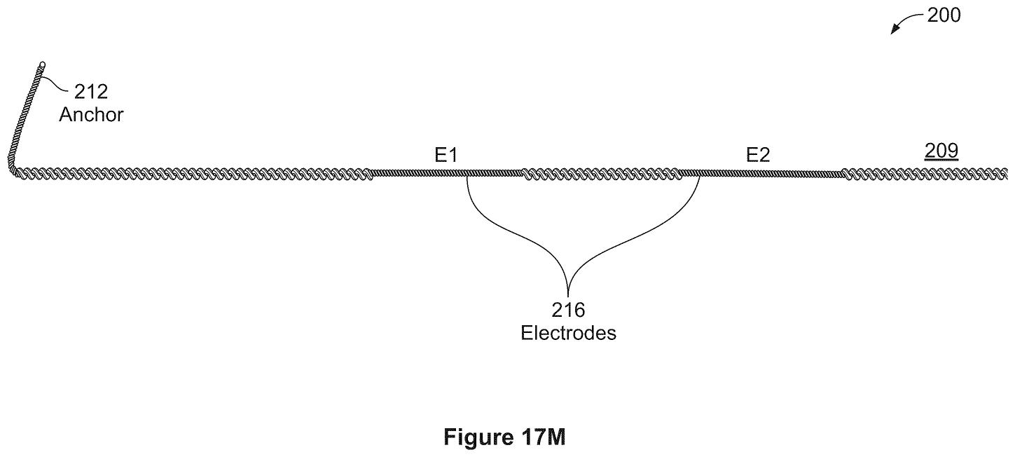

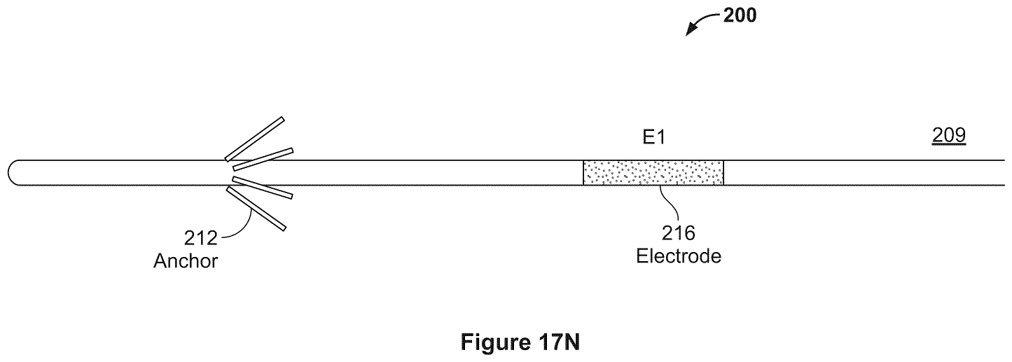

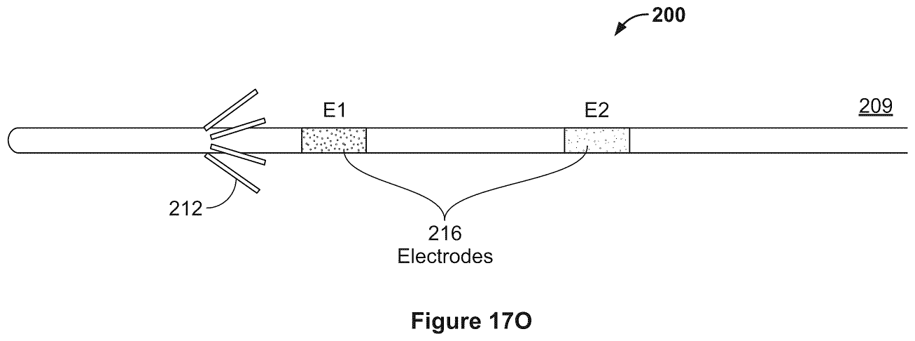

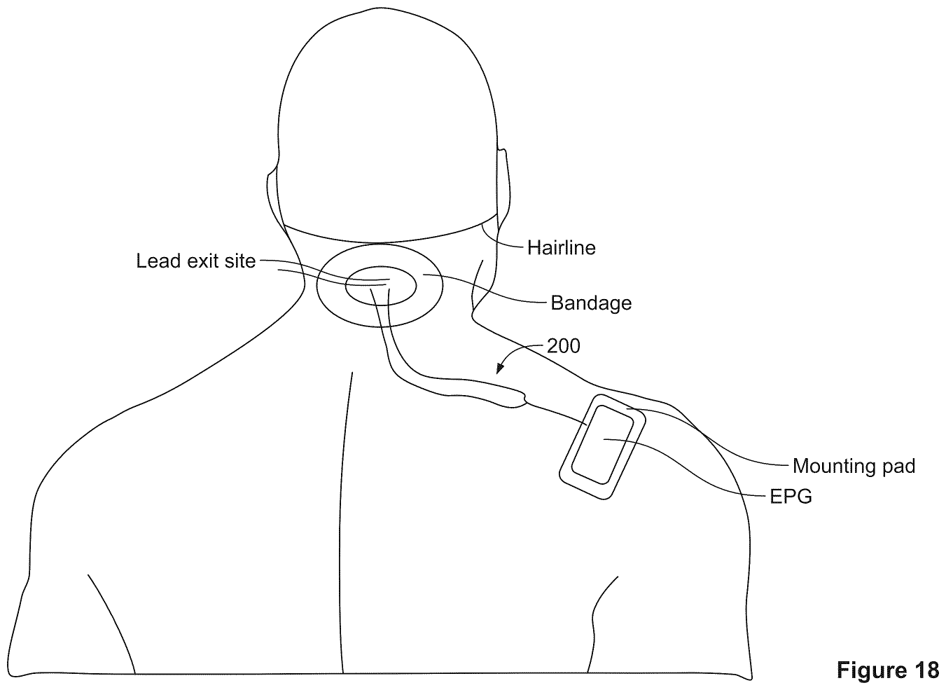

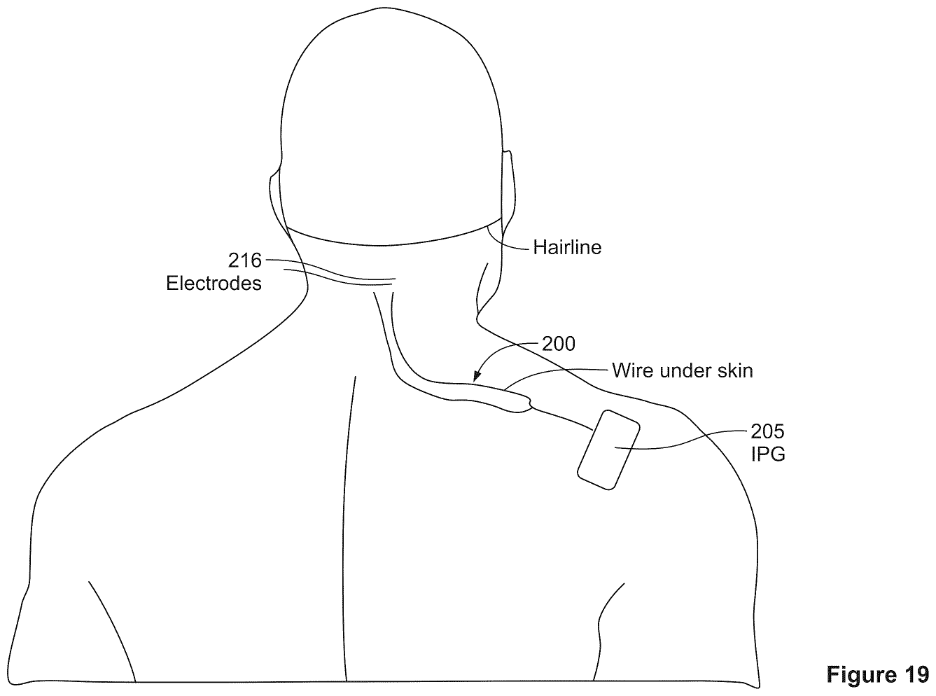

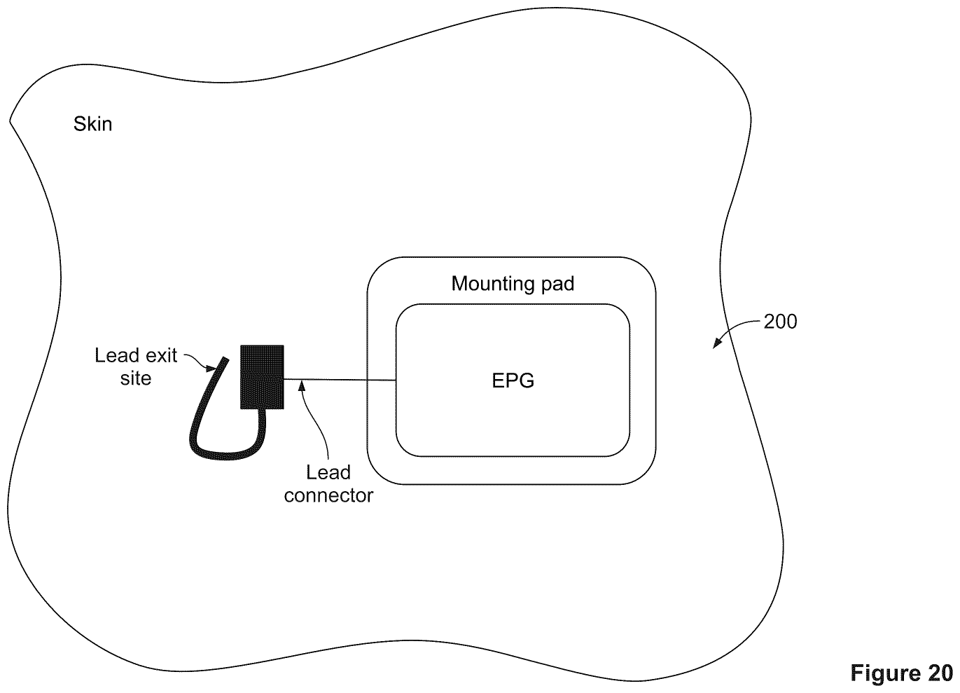

In an embodiment, the present invention teaches that it is desirable to place the lead in locations where the remote distance from the target nerve can be achieved while avoiding unintended activation of non-target nerve fibers, sensory endings, and/or other structures whose activation may be uncomfortable, painful, and/or non-desirable (FIG. 13A). As a non-limiting example, whereas conventional systems and approaches have placed leads distally (e.g., superior, above, or cephalad to the nuchal ridge or occipital prominence) on the occipital nerves in order to avoid the high mobility of tissues such as the musculature around the C1-C2 vertebral levels where the more proximal portions of the occipital nerves are located and these conventional systems and approaches have subsequently been limited by unintended or undesired activation of non-target nerve endings in the cutaneous or subcutaneous tissue, the present invention teaches that the leads should be placed more proximally (e.g., caudad, below, or inferior to the nuchal ridge or occipital prominence, over the C2 lamina, over the lateral edge of the C2 lamina, etc.) where there is sufficient tissue around the occipital nerves and sufficient tissue depth to maintain a remote distance from the target nerve and maintain sufficient distance from other nerves or nerve endings to avoid their activation and thereby avoid generating undesirable sensations, pain, or discomfort, while also overcoming the risks of lead migration, fracture, dislodgement through the use of a self-anchoring lead with a coiled structure that enables tissue integration (e.g., healthy tissue ingrowth into, around, near, the coiled structure, anchoring aspects of the lead, and/or the lead body) to reduce migration and has mechanical properties that are designed to withstand the forces placed upon the lead by stresses, motion, and movement of the tissues in the area of the occipital nerves (FIG. 13A). It is to be appreciated that placement of stimulating leads at the proximal portion of the occipital nerves requires consideration of the concomitant challenges of lead migration, fracture, and dislodgement that have limited previous systems and methods, as well as approaches for lead implantation that take into account the unique considerations and challenges inherent in the anatomy surrounding the occipital nerves such as the multiplicity of muscles (e.g., rotatores, semispinalis cervicis, semispinalis capitis, splenius cervicis, splenius capitis, multifidus, oblique capitis inferior, trapezius, and/or sternocleidomastoid muscles) in close proximity to the occipital nerves whose unintended motor activation is not desired and can cause discomfort and/or prevent pain relief from being achieved.

The present system and method enable the lead, in a non-limiting example, to be placed percutaneously in a non-intersecting path near the nerve to maintain an optimal (e.g., at least a minimum) distance between the lead and the nerve when the trajectories of the lead and nerve bring them in closest proximity. By maintaining a minimum distance (e.g., 5-30 mm distant from the target nerve, and in some embodiments:1-50 mm, 1-40 mm, 1-30 mm, 1-20 mm, 1-10 mm, 1-5 mm, 2-50 mm, 2-40 mm, 2-30 mm, 2-20 mm, 2-10 mm, 2-5 mm, 3-50 mm, 3-40 mm, 3-30 mm, 3-20 mm, 3-10 mm, 3-5 mm, 4-50 mm, 4-40 mm, 4-30 mm, 4-20 mm, 4-10 mm, 4-5 mm, 5-50 mm, 5-40 mm, 5-30 mm, 5-20 mm, 5-10 mm, etc. distant from the target nerve) and avoiding direct contact with and/or immediate adjacency to the nerve, the safety of the procedure is improved by reducing the risk of the lead and/or needle to place the lead coming into contact with or puncturing the nerve and/or vasculature near the nerve (e.g., in a neurovascular bundle). Keeping the lead at a minimum distance (e.g., a therapeutically effective distance) from the nerve also enables or promotes preferential activation of target fibers (e.g., large diameter sensory fibers in the target nerve) instead of or over non-target fibers (e.g., small diameter sensory and/or pain fibers in the target nerve), enabling and providing pain relief and other desirable patient outcomes and responses. In other non-limiting examples, it may be advantageous or desirable to place the lead in an intersecting path (e.g., pointed directly at a nerve), for example if non-intersecting trajectories are unfeasible due to surrounding anatomy and/or if the most desirable location for stimulation or the most effective pain relief is achieved on an intersecting trajectory.

The present system and methods mitigate the risk of lead migration and/or dislodgement by placing, deploying, and/or implanting the lead (e.g., a percutaneous and/or fully implantable lead) such that a sufficient and/or desirable length of the insulated portion of the lead (e.g., >1 cm, ≥1 cm, >2 cm, ≥2 cm, >3 cm, ≥3 cm, >3 cm, ≥3 cm, cm, >4 cm, ≥4 cm, >5 cm, ≥5 cm, 1-4 cm, 2-4 cm, 3-4 cm, 2-5 cm, 3-5 cm, 4-5 cm, 2-6 cm, etc.) is inserted into the body. This placing, deployment, and/or implanting of the lead at such length may cause, promote, enable, and/or facilitate a healthy, physiological response to the lead including tissue ingrowth into the open coil structure of the lead that produces, ensures, and/or contributes to secure placement of the lead while also reducing infection risk and reducing or eliminating ingress of contaminants (e.g., pathogens, germs, contaminants, etc.) at the lead exit site on the skin.

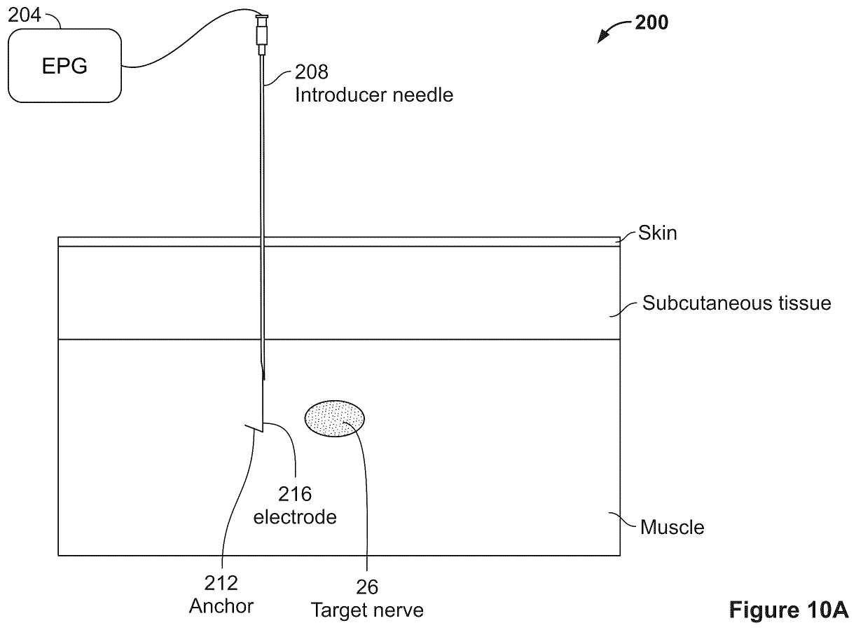

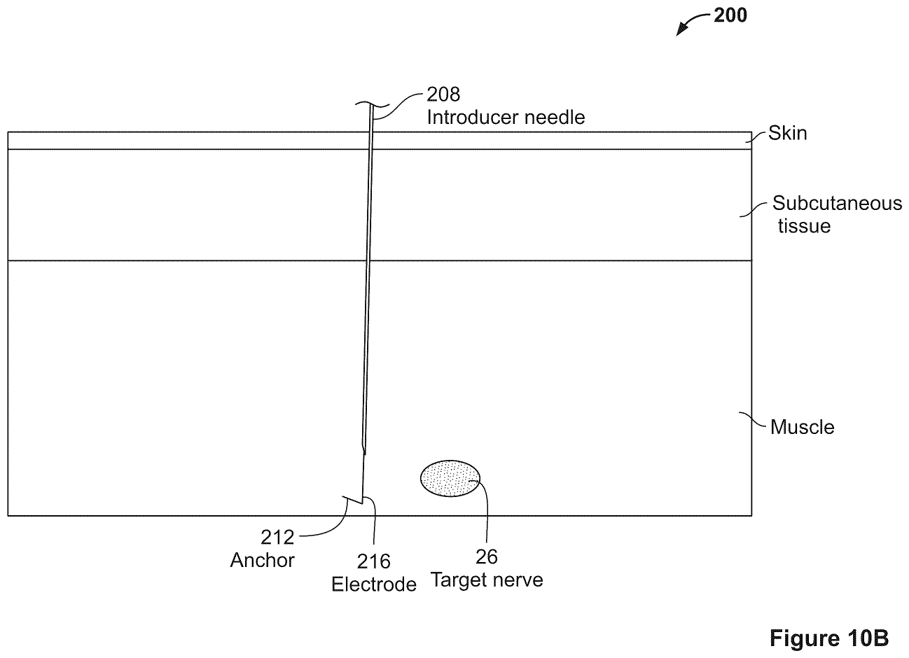

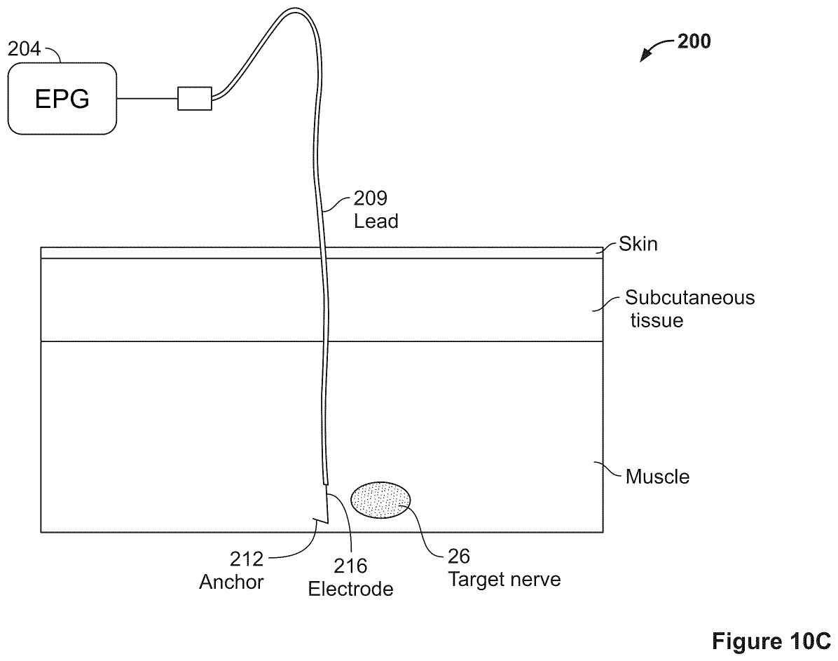

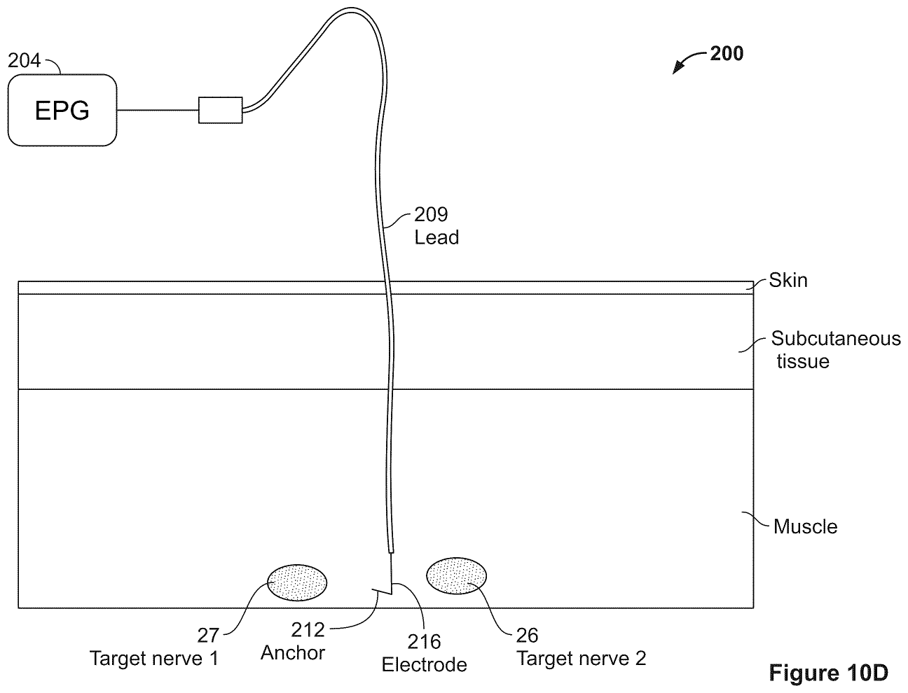

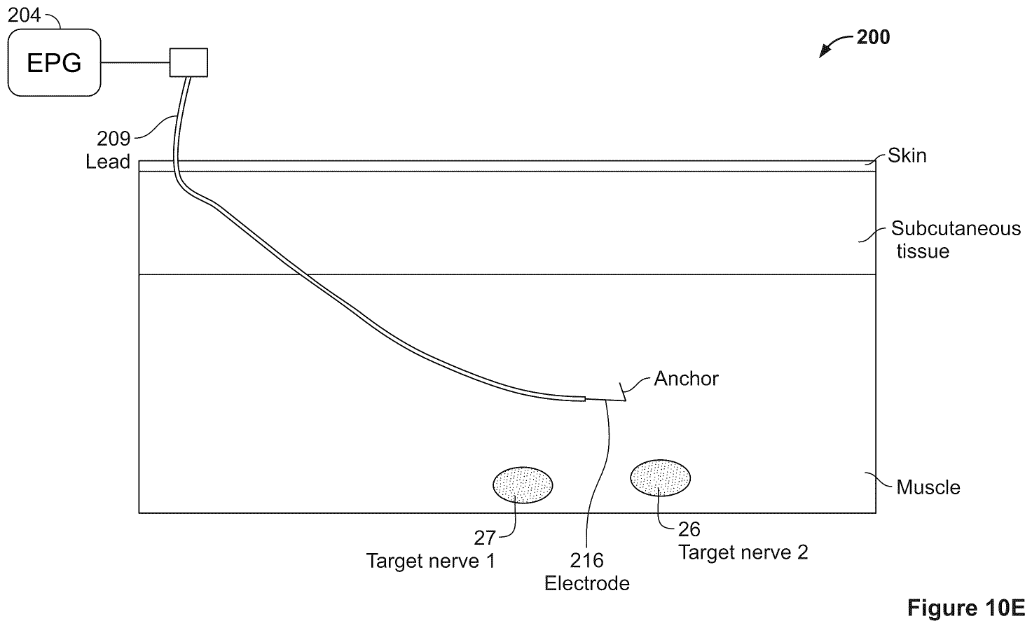

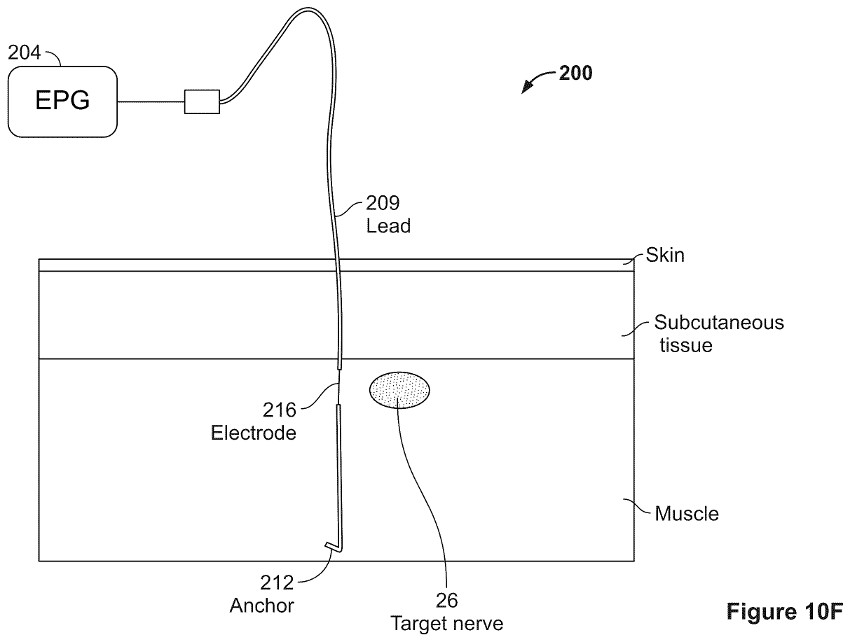

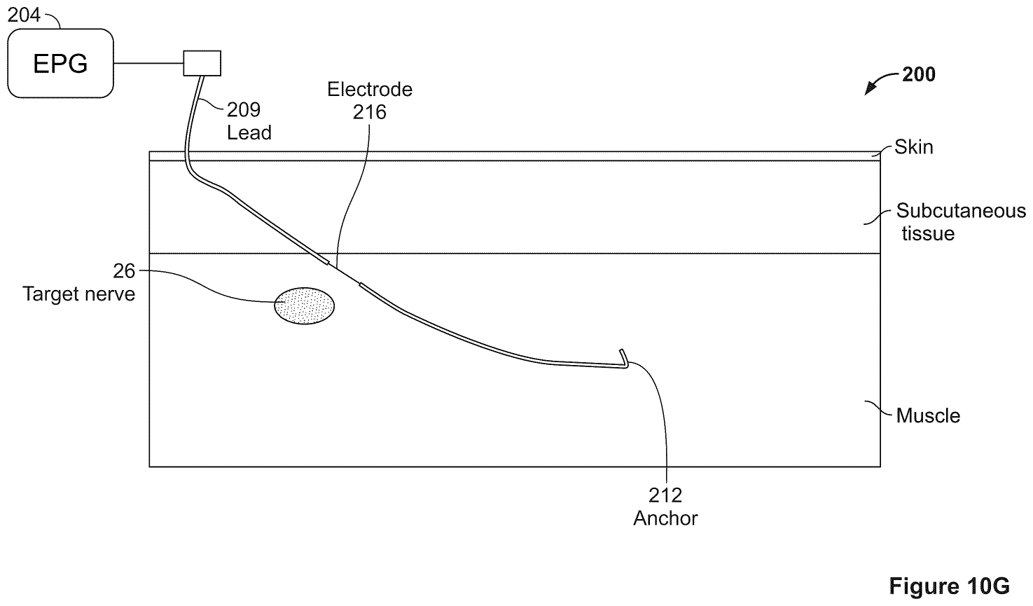

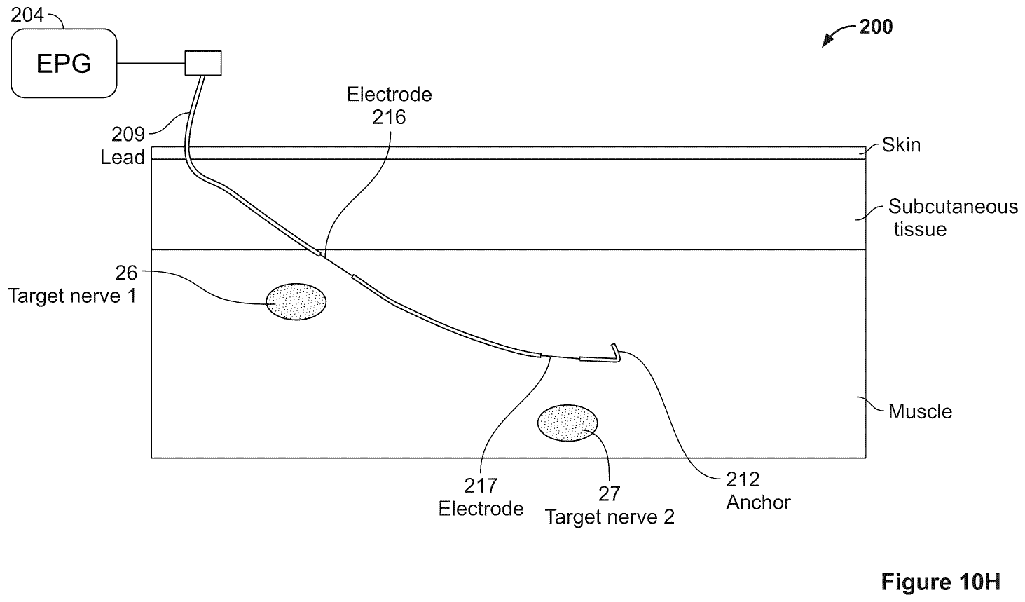

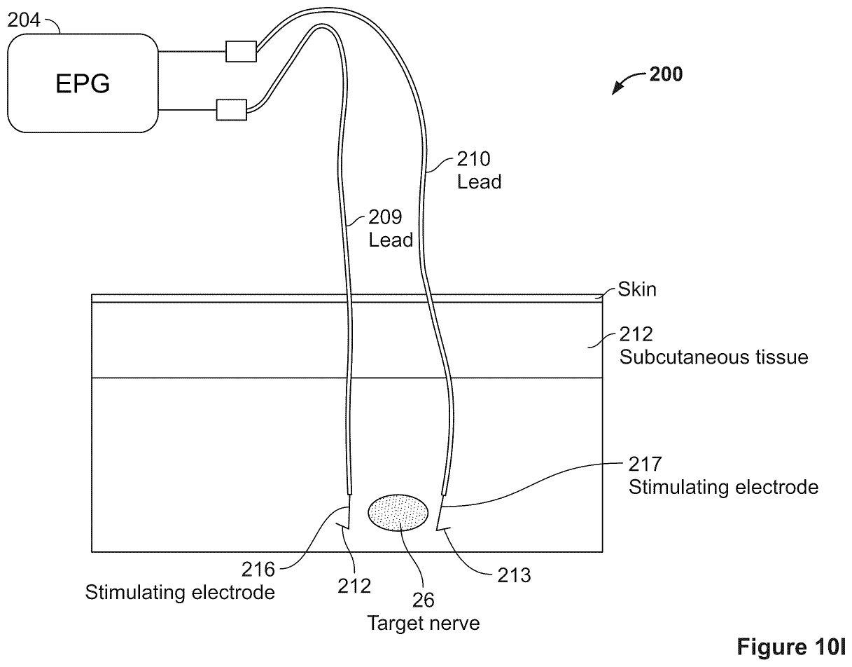

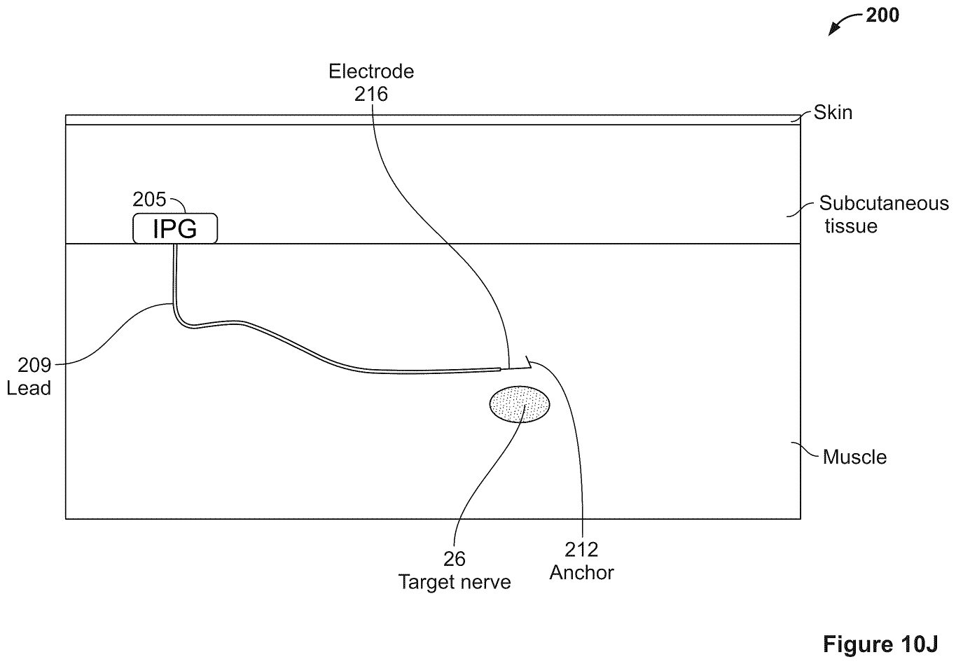

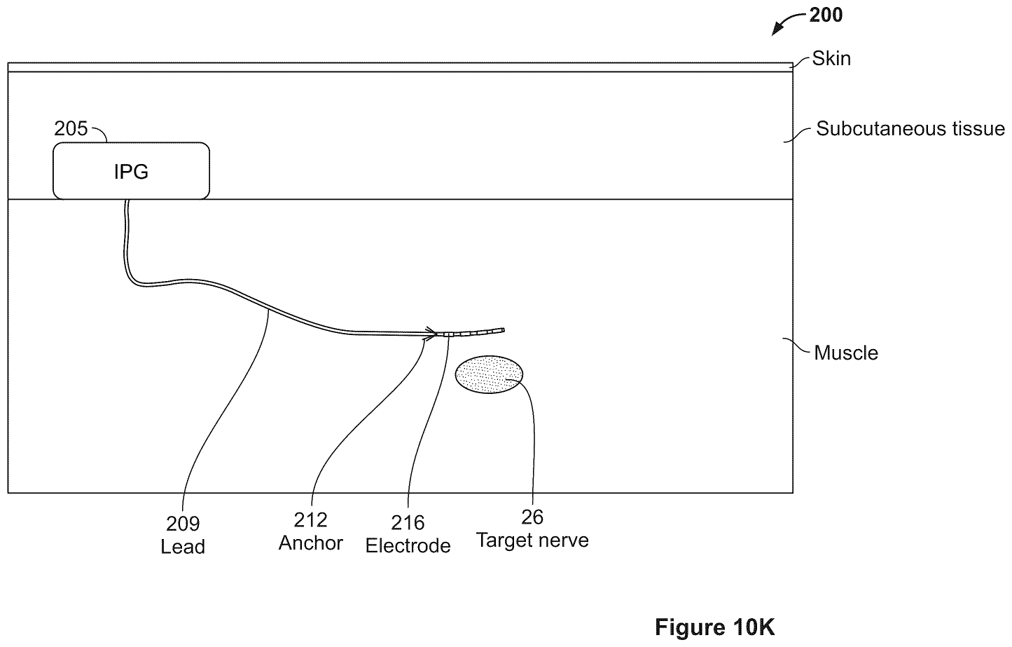

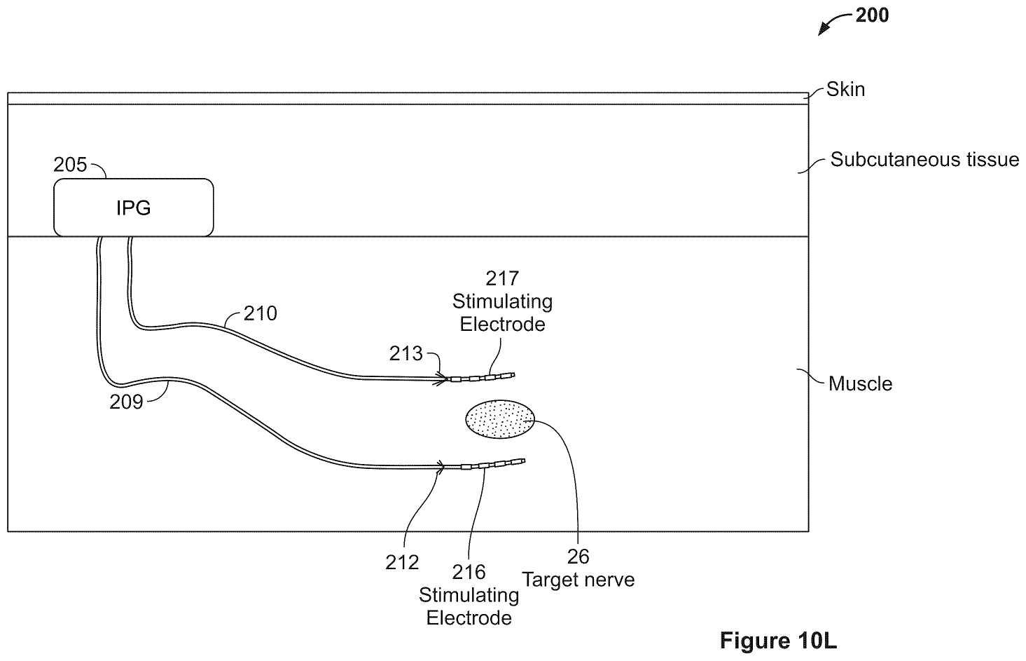

In an embodiment, the lead is a temporary lead placed percutaneously using a needle introducer and desired length of the lead is implanted in the body to enable at least 1-2 cm and preferably 3-4 cm of the coiled, insulated portion of the lead to be implanted percutaneously inside the body, with the remainder of the lead remaining external to the body (FIGS. 10A-0. In this non-limiting example, the insulated coiled structure of the lead enables, encourages, facilitates, and/or causes healthy, physiological tissue ingrowth (e.g., fibrotic ingrowth) that enhances the security and stability of the implanted lead such that the forces required to fully or partially dislodge the lead are increased and the accidental migration or dislodgement of the lead is avoided, prevented, and/or prohibited by the present invention and overall less likely to occur, while also preventing, avoiding, and/or reducing the risk of infection by preventing and/or avoiding small movements of the lead in and out at the entry site in the skin (e.g., “pistoning”) and thereby preventing, avoiding, and/or reducing ingress of contaminants at the lead entry site.

FIGS. 10A-I show examples using an external pulse generator 204 and a lead 209 that is implantable into a desired tissue region for electrical stimulation. As shown, the external pulse generator 204 and at least a portion of the lead 209 may remain external to the body while the remainder of the lead 209 and electrode 216 are implanted into the desired tissue region, which may be proximate to a target nerve 26. The lead 209 and electrode 216 may be implanted into the desired tissue region through use of an introducer needle 208. In an embodiment, the system 200 may include one or more components, such as an introducer and/or lead, or any other combination of described components that are configured to work with a stimulator, such as an internal or external pulse generator, in an example.

The electrode may be located on the distal end of the lead at, near, or adjacent the anchor 212 of the lead, or generally on a portion of the lead. As shown in FIGS. 10A-E, for example, the electrode may be positioned at or near the distal end of the lead. As shown in FIGS. 10F-G, the electrode may be positioned spaced apart from the distal end of the lead. The lead (having one or more than one electrode) may be used to stimulate a single target nerve, as shown in FIG. 10C, or the lead (having one or more than one electrode) may be used to stimulate different or more than one target nerve etc., see FIG. 10D.

As shown in FIG. 10H, the lead 206 may include more than one electrode 216, 217, etc. or a plurality of electrodes and, in an example, two electrodes. In an embodiment, the first electrode may be positioned at or near the distal end of the lead and the second electrode may be positioned spaced apart from the distal end of the lead (and the first and the second lead may be spaced apart from one another). In an embodiment, the first and the second electrode may be placed adjacent one another, spaced apart from one another, or generally placed anywhere on the implanted portion of the lead and anywhere relative one another. As shown in FIG. 10I, the device may include more than one lead or a plurality of leads 209, 210 and, in an example, two leads. The multiple leads may be attachable to the same external pulse generator, but may be implantable into different areas of tissue. The multiple leads may be used to stimulate the same target nerve, as shown in FIG. 10I, or the multiple leads my be used to stimulate different or more than one target nerve 26, 27.

The electrode may be positioned relative to the target nerve through the skin and subcutaneous tissue layers of the body and into muscle, in an example. An anchor 212 may assist in securing the lead and electrode and/or lead in a desired position. The anchor may be placed on the at or near the distal end of the lead, adjacent an electrode, on a part of the lead separate from the distal end, etc., for example, comparing FIG. 10J to FIGS. 10K-L. As shown in FIG. 10I, the device may include more than one anchor or a plurality of anchors 212, 213 (which can correspond to the number of leads and/or the number of electrodes) and, in an example, two anchors. After desired placement into the tissue, an external pulse generator may be connected to the lead(s) to apply external stimulation through the lead and electrode. As shown in FIG. 10A-I, the target nerve may be lower or higher within the muscle. As shown in FIG. 10A-I, the implantable lead may be placed in a straight or direct path to the target nerve from the surface of the skin, see FIGS. 10A-D, F, I or the implantable lead may be directed to the target nerve from an angle, see FIGS. 10E, G-H.

It is noted that system 200 may refer to and include any of the described components and any combination thereof, including one or more of electrodes, leads, anchors, introducer needles, pulse generators (internal and/or external) and the like.

In another embodiment, the lead is a fully implantable lead that does not have an externalized portion exiting the skin percutaneously during the treatment period and therefore does not have the risk of infection caused by having a percutaneously indwelling lead for the length of the stimulation treatment period. In this non-limiting example, the fully implantable lead has an insulated coiled structure that may cause, enable, facilitate, and/or permit healthy, physiological tissue growth, ingrowth (e.g., fibrotic ingrowth) and/or stabilization that enhances the security and stability of the implanted lead for the duration of the treatment such that the forces required to dislodge the lead anchor, lead tip, or other component of the lead are increased and the accidental migration of the lead is less likely to occur, enabling stable long-term stimulation treatment for patients who may desire or require continuous, extended, and/or indefinite stimulation treatment period(s) to maintain pain relief without requiring maintenance, bandaging, and/or care of a percutaneous, externalized lead and lead exit site (FIGS. 10J-L). In another non-limiting example, the fully implantable lead has an insulated structure that is not coiled but may still enhance the security and stability of the implanted lead for the duration of the treatment such that the forces required to dislodge the lead anchor, lead tip, or other component of the lead are increased and the accidental migration of the lead is prevented and less likely to occur, enabling stable long-term stimulation treatment for patients who may desire, need, benefit from and/or require continuous, extended, and/or indefinite stimulation treatment period(s) to maintain pain relief without requiring maintenance, bandaging, and/or care of a percutaneous, externalized lead and lead exit site (FIGS. 10J-L).

FIGS. 10J-L show examples using an implantable pulse generator 205 and a lead 209 that is implantable into a desired tissue region for electrical stimulation. As shown, the implantable 205 pulse generator and the entire lead 209 may be implanted into the body and/or the desired tissue region, which may be proximate to a target nerve 26. The lead and electrode may be implanted into the desired tissue region through use of an introducer needle 208. The implantable lead may have generally the same or similar aspects to the leads of the external pulse generator embodiments (and vice versa), including number and placement of leads, number and placement or electrodes, anchors, positioning relative to one another and target nerves, number of target nerves, etc.

The electrode may be positioned relative to the target nerve through the skin and subcutaneous tissue layers of the body and into muscle, in an example. The internal pulse generator may be positioned in the subcutaneous tissue of the body.

The anatomy of the occipital nerves and surrounding structures create unique anatomical challenges that limit the ability of previous and conventional systems and methods to achieve implantation of a sufficient or optimal length of lead under the skin (e.g. not too long or too short of a lead length) while also placing the stimulating electrode within a therapeutic and/or optimal distance from the target nerve(s) (e.g., not too close or too far from the target nerve(s) and/or nerve fiber(s) while also optimizing the distance from non-target and/or off-target nerve fibers, nerves, and/or fibers). As a non-limiting example, the present invention was designed and developed such that it could work in, within, and/or around the greater and third occipital nerves at the level of the C2 vertebra using the tissue, tissue depth, and/or surrounding tissue to enable placement of the electrode contact remote and/or within a therapeutic distance of the nerve(s) while also overcoming the challenges of previous or conventional system(s) and/or method(s), such as the challenges associated and/or caused by the nerves lying a short distance (e.g., 1-2 cm for the third occipital nerve, 2-3 cm for the greater occipital nerve) from the midline such that a lead insertion trajectory from the midline laterally towards the nerves such that previous or conventional system(s) and/or method(s) would not achieve sufficient (e.g., >3 cm, ≥3 cm, >4 cm, ≥4 cm, 3-5 cm, preferably at least 3-4 cm) length of lead implanted under the skin to enable healthy tissue growth in, around, near, surrounding (partially or completely) the lead, lead body, anchors, tines, and/or anchoring features, components, and/or elements. Previous systems and methods have attempted to overcome these anatomical challenges by a) inserting the needle contralaterally such that the lead crosses the midline and is forced to traverse between two spinous processes, which subjects the lead to additional challenges and problems (that the present invention overcomes), such as forces by crossing additional tissue planes, joints, and axes of movement in the cervical spine and therefore increases risks of lead migration, dislodgement, or fracture, or b) placing the lead to descend inferiorly along the lateral side of the spinous processes to avoid crossing the midline by adding one or more turns, curvatures, or corners to the lead path, which may act as concentration points for stress, strain, shear, or other mechanical forces on the lead that increase the risk of lead fracture. In an embodiment, the present system and method enable placement of the stimulating electrode within a therapeutic distance from the target nerve(s) while ensuring the implantation of a sufficient length of lead under the skin and avoiding crossing the midline or creating turns or corners in the lead trajectory through the use of a lead whose active, non-insulated portion of the lead (e.g., the electrode contact) is located a distance (e.g. 1-10 mm, 2-30 mm, 2-50 mm, 5-50 mm, 10-20 mm, 10-40 mm, etc.) from the distal tip of the lead such that the electrode is not at the most distal end of the lead (FIGS. 10F-G, 13H-J). In this non-limiting example, the lead may be inserted at an entry site that is within, closer than, and/or not distant enough from the target nerve to achieve a sufficient length (e.g., preferably at least 3-4 cm) of the coiled, insulated portion of the lead under the skin, advancing the distal end of the lead beyond the target nerve and placing the electrode contact within a therapeutic distance from the target nerve while the distal end is placed outside of a therapeutically effective distance from the target nerve, achieving a sufficient length of the coiled, insulated portion of the lead under the skin to provide stability and security to the implanted lead such that the invention prevents unwanted migration and/or dislodgement of the lead.

In another non-limiting example, such as that shown in FIG. 10H, the lead has two or more electrodes located along the lead separated by insulated and/or coiled portions of the lead, enabling stimulation through one or more electrodes that are within a therapeutic distance of one or more separate target nerves to enable activation of target fibers in one or more target nerves while avoiding activation of non-target fibers in the target nerves and/or activation of other non-target structures or nerve fibers and while enabling a sufficient length of the insulated, coiled portion of the lead to be implanted beneath the skin to promote healthy tissue ingrowth for stability and security such that the invention prevents unwanted migration and/or dislodgement of the lead. In an embodiment, the electrodes may both deliver electrical stimulation to the tissue surrounding the electrodes or each electrode may be selectively chosen to deliver electrical stimulation or different intensity or patterns of electrical stimulation as may be desired.

It is important that the lead be delivered to the ideal location within the neck without damaging the surrounding tissue to an extent that procedural and/or post-procedural pain attenuate, prevent, negate, offset, and/or overcome pain relief provided by the stimulation treatment, and therefore the present system and method prevent and/or minimize tissue damage through use of a lead whose diameter enables it to be delivered percutaneously using a needle and/or introducer without any incisions, open cut-downs, and/or surgical approaches. In a preferred embodiment, the lead is thin (e.g., ≤0.3 mm, >0.3 mm, 0.3-0.6 mm, 0.3-0.75 mm, 0.2-1.0 mm, <0.75 mm, <1.2 mm, ≤1.2 mm, and in a preferred embodiment ≤0.75 mm in diameter) enough to be delivered within a thin introducer (e.g. >14 gauge, 14-18 gauge, 15-20 gauge, >16 gauge, and in a preferred embodiment ≥17 gauge). The percutaneous delivery of the small-diameter lead to a therapeutically effective distance from the target nerve that is not immediately adjacent to (e.g., <1 mm, <2 mm, etc.) and/or in direct contact with the target nerve, especially when performed under fluoroscopic and/or ultrasound imaging (e.g., to visualize the lead, needle, target nerve, non-target nerve(s), vasculature, soft tissues, and/or bony landmarks), further mitigates, minimizes, prevents, and/or avoids the risk of nerve damage, vascular damage, and/or soft tissue damage from the lead placement.

Resistance to migration, fracture, and uncomfortable stimulation are important characteristics to provide pain relief through stimulation of the occipital nerves, and the present system and method overcome these challenges through the use of thin, flexible, migration-resistant leads coupled with lead placement approaches that navigate the complex anatomy, physiological challenges, stresses, and tissue environment around or near the occipital nerves. In a non-limiting example, the system and method include a stimulating lead that (a) stays anchored in the treatment location as the patient moves, (b) is resilient and/or flexible when the patient and/or the tissue moves, and (c) withstands the stress caused by the movement of the patient, anatomy, musculature, joints, and/or tissue. In the case where the stimulating lead is a temporary percutaneous lead intended to deliver a temporary stimulation treatment and intentionally removed or withdrawn at the end of the treatment period, the lead also (d) has a high enough withdraw force to stay in the patient for a temporary period of time (e.g., 15 days, 30 days, 60 days, 2 months, 3 months, multiple months, one or more years, multiple years, etc.), and (e) has a low enough withdrawal force to be removed without surgical intervention, without causing undue discomfort to the patient, and without fracturing the stimulation lead. The lead is designed to be sufficiently flexible and/or not too stiff because if the stimulation lead is too stiff, it will not move easily with the patient, putting stress on keeping the stimulation lead anchored and resulting in migration, dislodgement, or skin erosion (e.g., migration through the skin). At the same time, the lead is also designed to be sufficiently stiff and/or not too flexible because on the other hand, if the stimulation lead is not stiff enough, the over flexing of the stimulation lead can promote stress fracturing. For example, the present system and method overcome the limitations of prior occipital nerve stimulation systems and approaches wherein stimulation leads were often placed in the subcutaneous space in close proximity to and/or in direct contact with the occipital nerves resulting in challenges with skin erosion due to the lack of lead flexibility coupled with placement in the thin layers of cutaneous and subcutaneous tissues overlying the occiput (e.g., cranial bones or posterior skull), and/or lead migration or lead fracture from repeated stress and strain on large conventional non-flexible leads.

In particular, whereas conventional systems and methods use relatively large, (e.g., 1.2-1.4 mm diameter) stiff, inflexible leads that produce a risk of skin erosion from repetitive movements of the lead and/or the subcutaneous and cutaneous tissue overlying the lead, the present system and method in a preferred embodiment utilize thin (e.g., ≤0.3 mm, >0.3 mm, 0.3-0.6 mm, 0.3-0.75 mm, 0.2-1.0 mm, <0.75 mm, <1.2 mm, ≤1.2 mm, and in a preferred embodiment ≤0.75 mm in diameter), flexible open-coil and/or closed-coil leads whose lack or avoidance of too much stiffness and/or rigidity (e.g., sufficient flexibility, malleability, pliability, softness, etc.) avoid the risk of skin erosion even when leads are placed subcutaneously such as in shallow locations superior to the nuchal ridge where there is minimal tissue for lead placement between the cranial bone and subcutaneous tissue layers. As a further example, it is also possible for lead(s) to be too flexible, malleable, pliable, and/or soft, and the present invention is sufficiently strong, stiff, and/or retains its shape, position, and mechanical and electrical integrity sufficiently to prevent or avoid unwanted fracture, movement, migration, or other failures. In other words, the system and approach is designed to enable desirable movement of the lead and/or system with the tissue (e.g., maintaining its correct and effective location and distance relative to the nerve target(s) and non-target(s), off-target(s), musculature, skin, connective tissue, bones, joints, and other anatomical structures) while avoiding or preventing unwanted movement that could cause failure of the system, preventing unwanted or adverse effects or events (e.g., to the system and/or to the patient or tissue), and/or preventing loss of effective treatment.

Simply substituting a more flexible lead for a less flexible lead cannot be expected to provide all these benefits, and the lead path, trajectory, and approach, as well as the electrode location and other features and methods are also considered in the present invention to enable lead securement throughout the stimulation period while avoiding uncomfortable stimulation of non-targeted fibers in the target nerve and/or in other structures. The present system and method enable and/or facilitate the stimulation lead to be placed, implanted, and/or deployed such that the stimulating electrode avoids stimulating and/or activating non-target fibers such as pain fibers, nerve endings, motor fibers in the muscles surrounding the target nerve, or other non-target sensory structures in the cutaneous or subcutaneous tissues during, concurrent with, or before (e.g., at lower levels or intensities of stimulation) the activation of desired target fibers in the target peripheral nerve. As a non-limiting example in contrast to previous or conventional approaches, one potential embodiment of the present invention avoids need for or the limitation of the positioning of the stimulation electrode superior to the nuchal ridge (e.g., superior to the inion or the occipital protuberance), to avoid unintentionally activating non-target fibers such as pain fibers, nerve endings, or other non-target sensory structures in the cutaneous or subcutaneous tissues. In another non-limiting example, the present invention also enables and/or facilitates the stimulation lead to be placed, implanted, and/or deployed such that stimulation preferentially activates target fibers (e.g., large diameter sensory fibers in the target peripheral nerve, such as the greater, lesser, and/or third occipital nerves) while avoiding unintended and/or undesirable activation of motor fibers in the one or more muscles surrounding the target nerve (e.g., rotatores, semispinalis cervicis, semispinalis capitis, splenius cervicis, splenius capitis, multifidus, oblique capitis inferior, trapezius, or sternocleidomastoid muscles) whose activation may generate unintended and/or undesired motor activity, contraction, elongation, rotation, etc. of one or more muscles or muscle groups in the upper cervical spine or posterior head and neck.

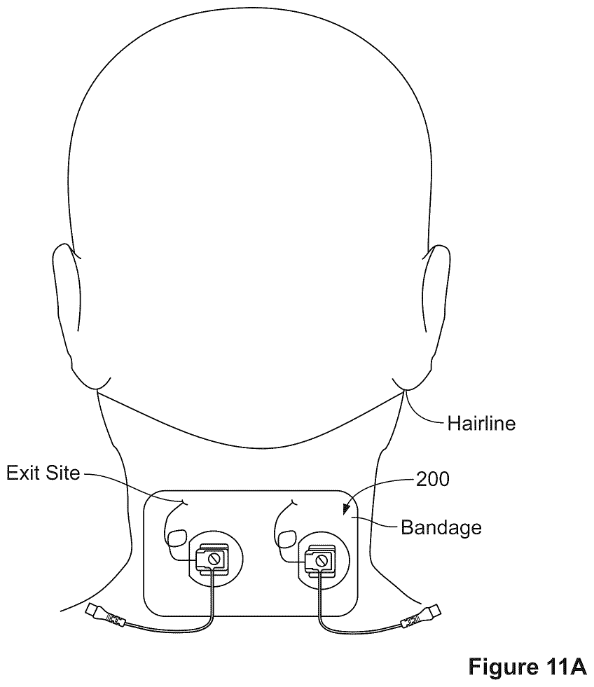

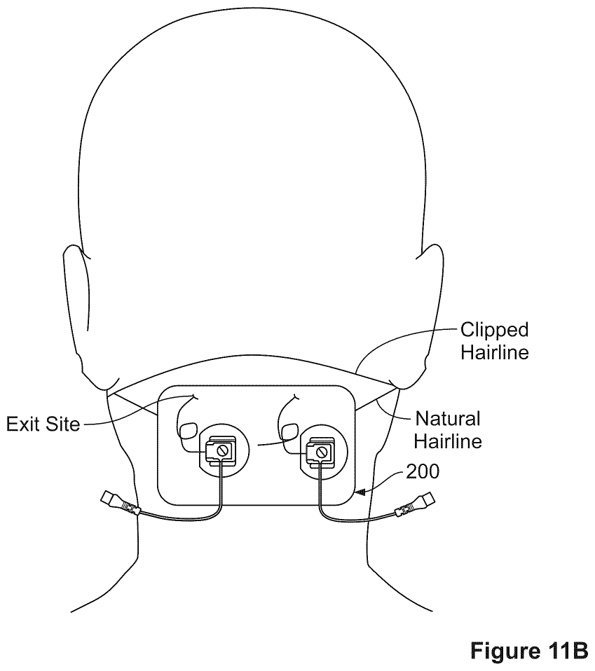

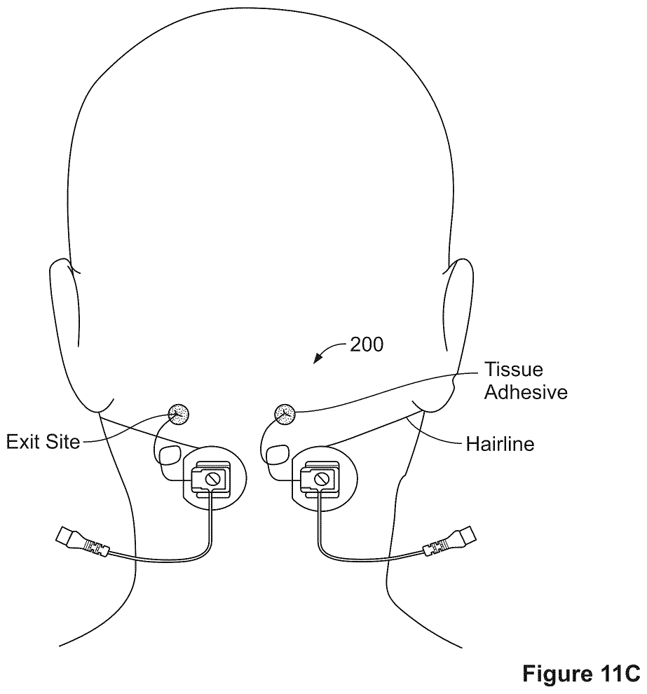

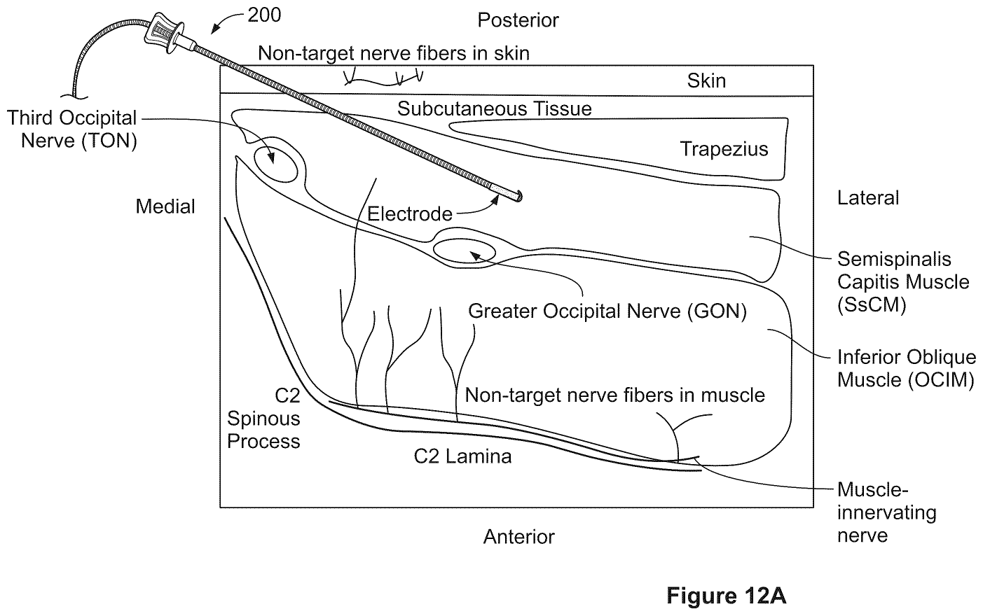

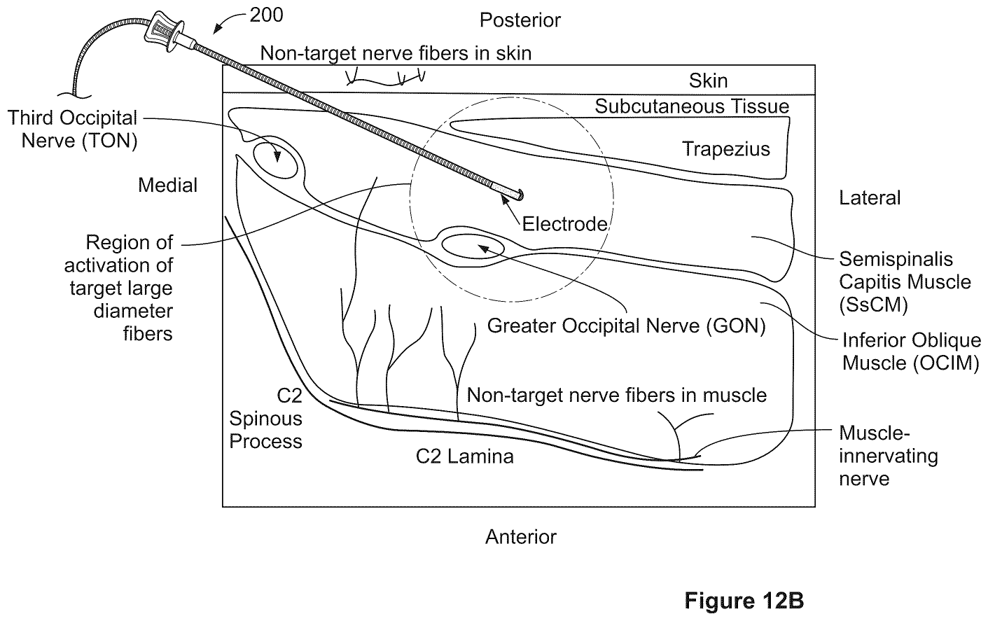

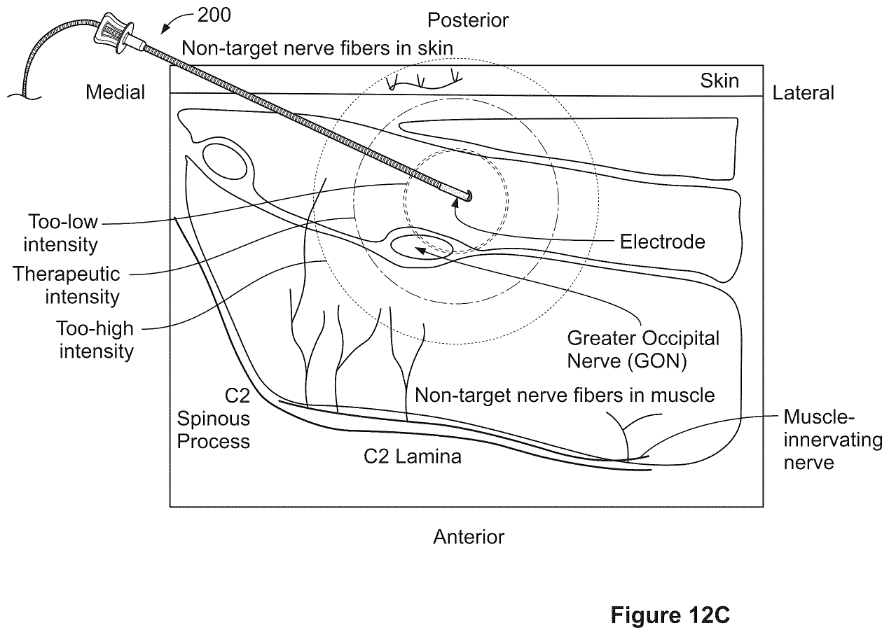

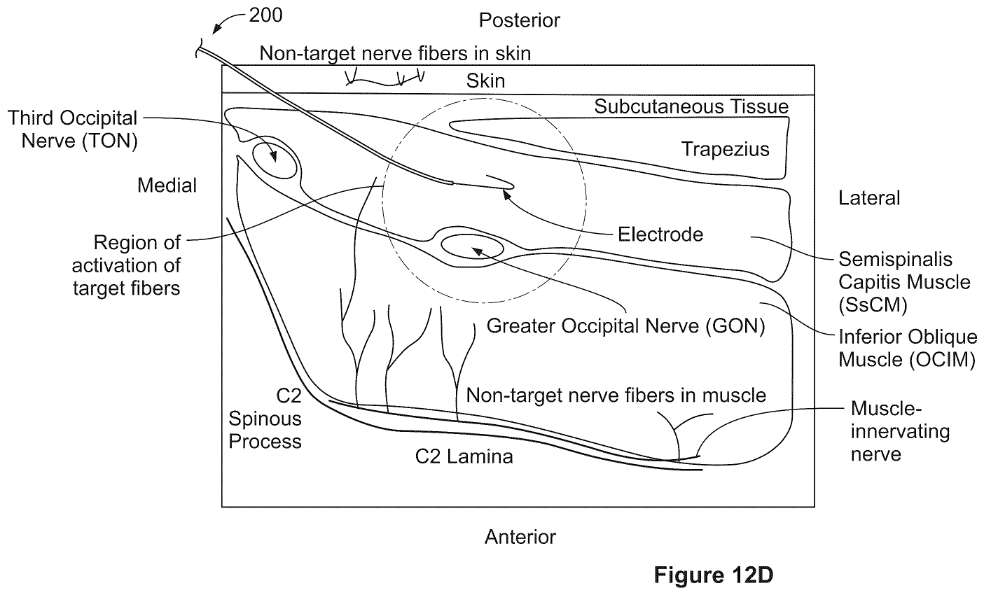

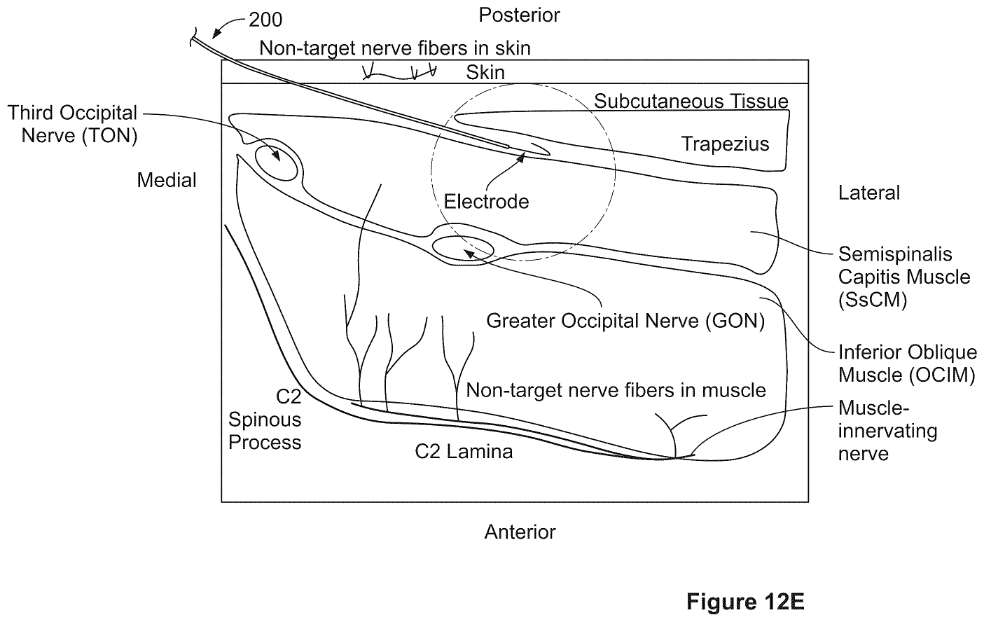

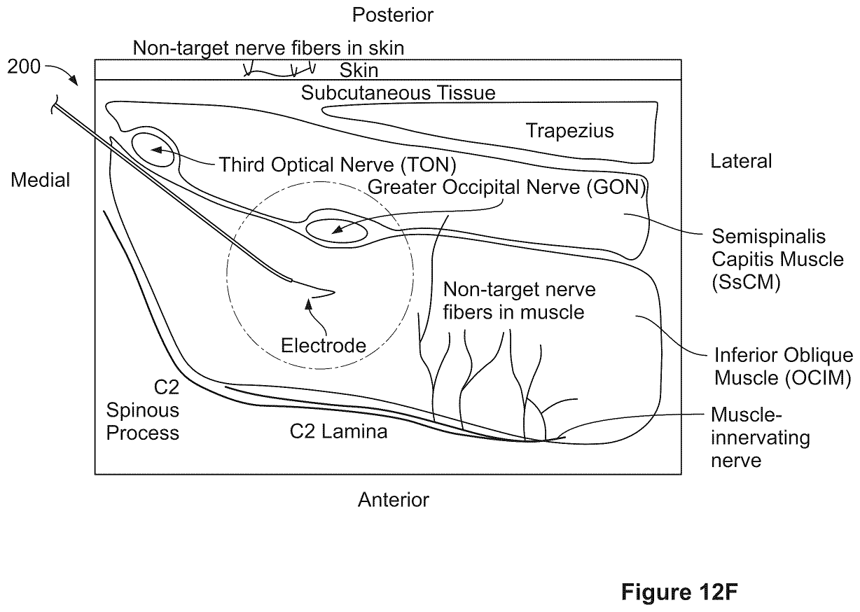

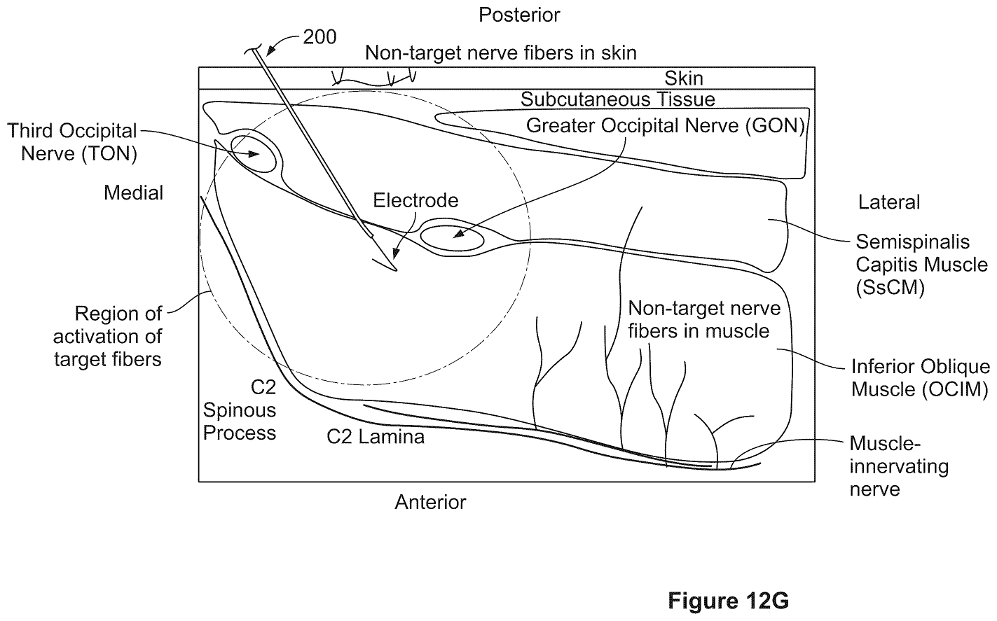

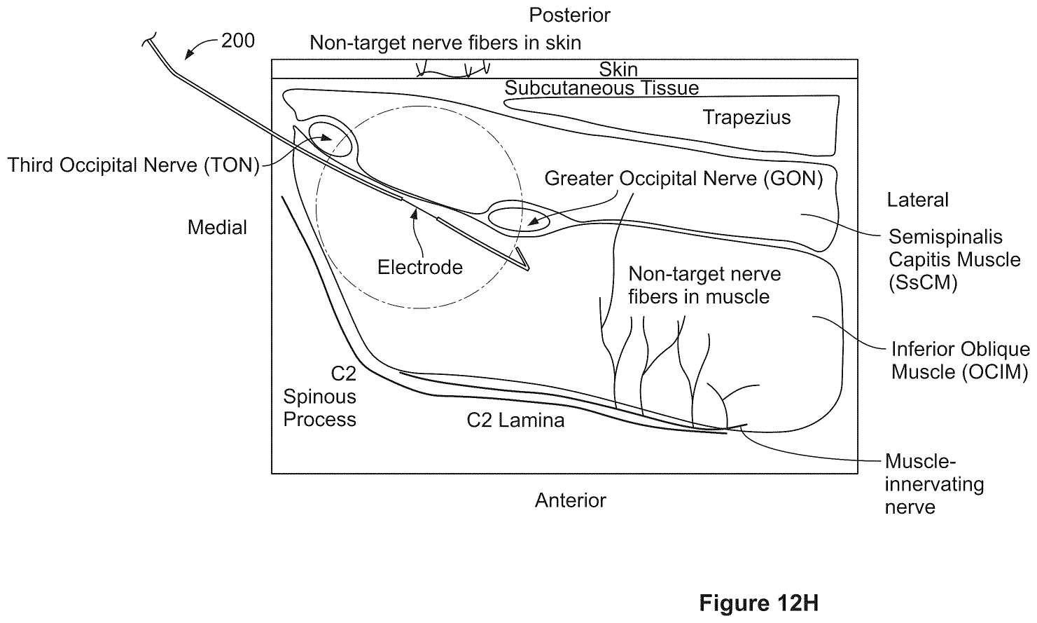

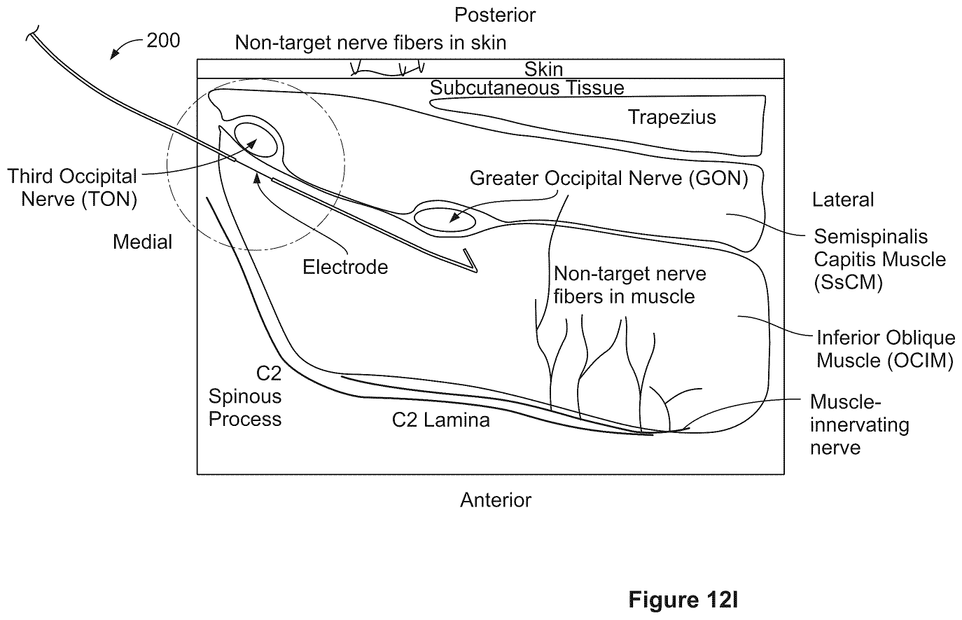

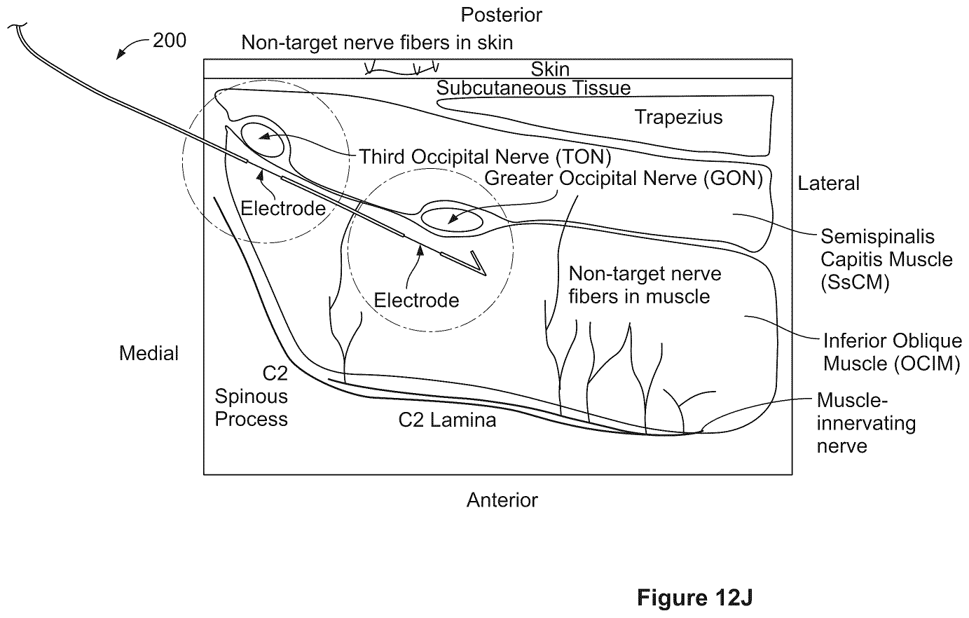

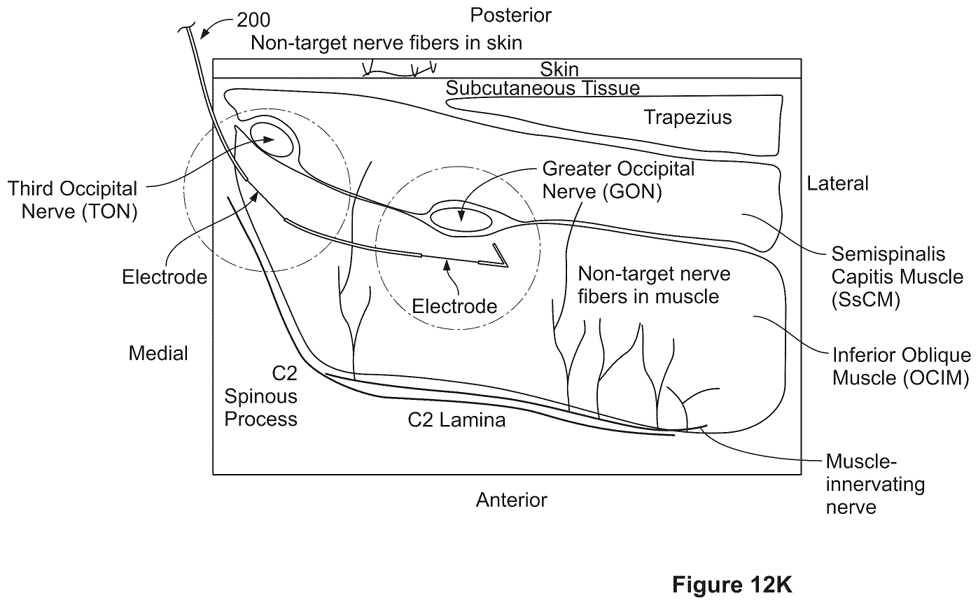

Furthermore, in the non-limiting example of a temporary percutaneous lead, the present system and method enable and/or facilitate placement of the stimulation lead such that the location of the exit site of the temporary percutaneous lead, that is, the location on the skin where the lead is inserted and where the externalized portion of the lead exits the skin throughout the temporary stimulation period, is conducive to the application of bandaging materials that protect the exit site from contamination and avoid or reduce infection risk during the temporary stimulation or treatment period. In a preferred embodiment (FIG. 11A), the exit site of the percutaneous stimulating lead is located inferior (e.g., >2 cm, >3 cm, ≥3 cm, 3-4 cm, 2-5 cm, >5 cm, etc. below) the hairline of the patient, and ideally at least 3-4 cm below the hairline to enable the application of a bandage around the exit site without requiring modification to the hairline (e.g., hair cutting, clipping, etc.). In another embodiment, the exit site is located at or below the hairline and some portion of hair may be cut or clipped to enable placement of a bandage over the exit site of the percutaneous stimulating lead while avoiding adhesion of the bandage to the hair, and the hair may be re-clipped or re-cut periodically during the temporary stimulation period to enable ongoing adhesion of the bandage to the skin (FIG. 11B). In another embodiment, the exit site is placed at or above the hairline and the hair may be clipped, cut, or removed to facilitate the use of an adhesive bandage, and/or non-fabric adhesive bandages (e.g., liquid bandage, skin glue, tissue adhesive, cyanoacrylate, 2-octyl cyanoacrylate, Dermabond, etc.) may be used to seal the exit site and protect against infection without requiring the hair to be fully or partially removed, clipped, or cut (FIG. 11C).

In an embodiment, the percutaneous introducer needle that delivers the stimulating lead to its target location shall not be curved, bent, or angled in order to avoid compromising the deployment of the lead from the introducer needle by the curvature of the introducer and to avoid tunneling and/or inserting the lead along a path that has one or more corners or turns that may act as concentration points for stress, strain, shear, and/or other mechanical forces on the lead that could increase the risk of lead fracture. In an alternate embodiment, the introducer needle is sized and configured to be bent before, during, and/or after manufacture (e.g., manually, by the user, and/or by hand) prior to its insertion through the skin or it is manufactured and/or configured post-manufacturing in a bent configuration without compromising the placement, deployment, and/or implantation of the lead such that the physician may place the lead(s) in a location that is not in an unobstructed straight line with the insertion site and/or along a trajectory whose curvature does not increase the risk of lead fracture (e.g., by following the natural curvature, boundary, or plane of one or more anatomical features, structures, tissue layers, tissues, muscles, muscle groups, bones, nerves, vessels, etc.). In this embodiment, the construction and materials of the introducer needle allow bending without interfering with the deployment of the lead(s) and withdrawal of the introducer needle, leaving the lead(s) in the tissue at the targeted and/or desired location following deployment (e.g., withdrawal of the introducer needle). In a non-limiting example, the system may be designed such that the introducer needle and/or other implantation and/or lead introducer tools are curved sufficiently to enable placement of the lead subcutaneously along the anatomical curvature of the neck, but the curvature is limited and/or designed to avoid and/or prevent sharp turns in the lead path that could be points of accumulation for stress, strain, shear, and/or other mechanical forces on the lead that could increase the risk of lead fracture and also remains straight enough to deploy the lead smoothly and without obstruction or incidental migration to the targeted location (e.g., straight enough to avoid the coiled structure of the lead kinking, catching, and/or otherwise becoming obstructed or prevented from deploying from the introducer needle upon withdrawal of the needle).

In a non-limiting example, the present system and method use a curved introducer to overcome the challenge of placing sufficient lead length beneath the skin to secure the lead in place when using a medial to lateral approach toward the greater occipital nerve at the level of the C2 vertebra, where the greater occipital nerve lies approximately 2 cm deep and approximately 3 cm from the midline. While in other areas of the body there may be straight-line approaches that enable placement of an electrode within therapeutically effective distance from a target nerve while also enabling implantation of a sufficient length of lead under the skin, the bony anatomy of the spine near the midline and the shallow location of the occipital nerves create a unique challenge to inserting the lead along a straight line trajectory or path, which the present invention addresses through the use of an introducer needle and/or other implantation tools that are configured to be bent before, during, and/or after manufacture (e.g., manually, by the user, and/or by hand) to match the desired curvature to place the stimulating electrode within a therapeutically effective distance from the nerve while enabling a sufficient length of the lead to be implanted under the skin.

In an embodiment, the introducer must be able to bend enough to accommodate the curvature of the neck and/or one or more component structures or tissues in the neck while maintaining its integrity such that it does not break, fracture, or otherwise impede the delivery or deployment of the lead when the introducer is removed. In one non-limiting example, the introducer has sufficient curvature and/or is sized and configured to be bent before, during, and/or after manufacture (e.g., manually, by the user, and/or by hand) with a radius of curvature (e.g., >2 cm, ≥2 cm, >2.5 cm, ≥2.5 cm, >3 cm, ≥3 cm, 2-5 cm, ≥5 cm) that enables the introducer to be inserted through the skin near the midline ipsilateral (e.g., on the same side of the midline) to the target nerves, advanced along a curved trajectory anterior (e.g., deep to or below) or posterior (e.g., superficial to or above) the GON and/or TON such that the stimulating electrode is within a therapeutically effective distance from the GON and/or TON, but the introducer is sufficiently straight and/or the radius of curvature is large enough that the lead path does not turn more than 90 degrees and/or does not include other sharp turns or bends that would create challenges with intentional removal of the lead at the end of a temporary stimulation period or that could be points of accumulation for stress, strain, shear, and/or other mechanical forces on the lead that could increase the risk of lead fracture. In another non-limiting example, the introducer has sufficient curvature and/or is sized and configured to be bent before, during, and/or after manufacture (e.g., manually, by the user, and/or by hand) with a radius of curvature that enables the introducer to be inserted through the skin one or more cervical spinal levels caudad (e.g., inferiorly, lower than) the target nerve(s), advanced along a curved trajectory cephalad (e.g., superiorly, towards the head) that follows the natural curvature of the cervical spine when in a neutral, flexed, or extended posture, such that the stimulating electrode is located within a therapeutically effective distance from the GON and/or TON at the level of the C2/C3 facet joint, at the C2 lamina, above the C2 lamina, and/or the nuchal ridge, but the introducer is sufficiently straight and/or the radius of curvature is large enough that the needle path and/or trajectory may be sufficiently long to enable location of the lead exit site below the hairline to facilitate bandaging without curving too far in any direction so as to place the electrode outside of a therapeutically effective distance from the target nerve.

Based on the teachings above, the present system and method include one or more approaches targeting the greater, lesser, and/or third occipital nerves, non-limiting examples of which are provided below.

In one embodiment, the lead is placed targeting the greater occipital nerve (GON) and/or the third occipital nerve (TON) at the level of the C2 lamina using a mediolateral (e.g., medial-to-lateral or lateral-to-medial approach) such that the lead is placed in the mediolateral plane, from a medial location to a more lateral location or from a lateral to a more medial location. In a preferred embodiment, the lead is placed in a medial-to-lateral approach (e.g., as opposed to lateral-to-medial) because it avoids the challenges of placing the lead along the lateral curvature of the neck where the insertion could be obstructed by the mastoid bone or the ear and/or the exit site could be near the ear where bandaging is difficult due to the ear itself and/or the hairline. In this preferred embodiment, the lead is placed starting at the midline at the level of the C2 lamina in a medial to lateral trajectory using a shallow angle (e.g., <20 degrees, <30 degrees, <45 degrees) such that the stimulating electrode lies remote (e.g., 0.5-1 cm) from the GON and/or the TON. However, it is to be appreciated that any appropriate angle of insertion may be used that enables the placement of the introducer needle and/or stimulating lead in its target location and/or trajectory, and in some cases a non-shallow and/or steep angle may be used or required such as if the target nerve is deeper (e.g., in patients with more overlying musculature and/or fat tissue) and/or more medially located based on anatomic variation. It is to be appreciated that this approach may include placement of the stimulating lead in a trajectory that is not exclusively in the mediolateral plane, and the lead may also and/or instead take a superolateral or inferolateral approach (e.g., at an angle from medial to lateral in the superior or partially superior direction, or from medial to lateral in the inferior or partially inferior direction), or a superomedial or inferomedial approach (e.g., at an angle from lateral to medial in the superior or partially superior direction, or from lateral to medial in the inferior or partially inferior direction).

As a non-limiting example, the stimulating lead may be placed such that one or more electrode contacts are located above or superficial to the GON and/or the TON in the fascial plane between the trapezius muscle and the subcutaneous tissue, in the trapezius muscle, in the fascial plane between the trapezius muscle and the semispinalis capitis muscle, in the semispinalis capitis muscle, adjacent (e.g., medial or lateral) to the GON and/or the TON in the fascial plane between the semispinalis capitis muscle and obliquus capitis inferior muscle, or below (e.g., deep to) the GON and/or the TON in the obliquus capitis inferior muscle, and the electrode(s) in each of these locations are within a therapeutically effective distance from the GON and/or the TON such that stimulation activates targeted fibers in the GON and/or the TON while avoiding activation of non-targeted fibers, avoiding activation of non-target fibers (e.g., pain fibers) in the cutaneous and subcutaneous layers, and avoiding the activation of muscles, muscle groups, muscle fibers, and/or efferent (e.g., motor) nerve fibers in the area of stimulation. While previous systems and methods include leads whose electrodes are either located solely at the distal end of the lead or whose leads include multiple electrodes arrayed along the lead (including at the distal end of the lead), the present invention includes in an embodiment a lead with one or more electrode contacts located a distance from the distal tip of the lead such that the distal tip of the lead is advanced from the midline laterally beyond the GON and/or the TON, achieving a sufficient length (e.g., preferably at least 3-4 cm) of the coiled, insulated portion of the lead under the skin to enable healthy tissue ingrowth while the one or more electrodes are located within a therapeutically effective distance from the GON and/or the TON. In this embodiment, the lead does not include an electrode contact at the distal end of the lead because the distal end is outside a therapeutically effective distance from the target nerve(s) and stimulation at that location is likely to only activate non-target or off-target structures (e.g., non-target nerves, nerve fibers, muscles, etc.). In non-limiting examples, the lead may include one or more electrodes targeting the GON, one or more electrodes separately or concurrently targeting the TON, and/or one or more electrodes separately or concurrently targeting both the GON and the TON (e.g., the electrode(s) are within a therapeutically effective distance of both nerves).

In other variations of this embodiment, the lead is placed starting from the contralateral side (e.g., the opposite side, across the midline) and proceeding across the midline medial to lateral such that a greater or sufficient length of the lead is inserted under the skin to enable health tissue ingrowth that enhances the security and stability of the lead. While previous or conventional systems and methods employing this approach have suffered from high rates of lead migration, fracture, and/or erosion of the lead and/or other system components through tissues and/or skin due to the crossing of the midline which may introduce additional mechanical forces, loading, crossing of tissue planes and/or joints, or other sources of stress, strain, and/or shearing of the lead, the present system and method employ a flexible, self-anchoring, migration-resistant, and fracture-resistant lead whose durability and mechanical properties in terms of flexibility and fatigue life enable it to be placed in a trajectory that crosses the midline. In one non-limiting example of this embodiment, if two leads are being placed bilaterally targeting the left and right GON, the leads may cross at the midline and the method teaches that the leads should be placed such that the introducer needles are on non-intersecting trajectories and a lead is not unintentionally contacted, damaged, bisected, or fractured by the introducer needle of the second lead. In another non-limiting example of this embodiment, if two leads are being placed unilaterally targeting the GON and the TON on one side, the leads may start contralaterally (e.g., on the opposite side of the target nerve) and advance in parallel or non-parallel trajectories towards their respective target nerves (FIG). It is to be appreciated that this approach includes placement of the stimulating lead in a trajectory that is not exclusively in the mediolateral plane, and the lead may also and/or instead take a superolateral or inferolateral approach (e.g., at an angle from medial to lateral in the superior or partially superior direction, or from medial to lateral in the inferior direction). It is also to be appreciated that this approach may occur at the level of the C2 lamina, but may also occur above or below the level of the C2 lamina, such as at the C3 lamina, between the C2 and C3 lamina, at the C2/C3 facet joint, at the level of C1, etc.

In some variations of the present invention, the lead is placed to target and/or activate the GON with one or more electrodes and also targets and/or activates the third occipital nerve (TON) and/or the lesser occipital nerve (LON) with one or more electrodes (e.g., the same electrode(s) and/or different electrode(s) than the electrode(s) targeting and/or activating the GON), producing comfortable sensations and/or pain relief in a larger region that includes the innervation regions of GON, and/or TON, and/or LON, and/or one or more regions outside the innervation region of the occipital nerves (e.g., trigeminal nerve via interaction with the occipital nerves in the trigeminocervical complex) such as in some instances of cervicogenic headache, neuralgia, or other headache or head and neck pain conditions where the pain may occur and/or be contributed to by one or more of GON, TON, and/or LON, and activation of TON and/or LON in addition to GON is desirable. In a non-limiting example, one or more leads, electrodes, and/or contacts target the GON and/or TON where both GON and TON lie between the trapezius muscle and the semispinalis capitis muscle, and the one or more leads, electrodes, and/or contacts may be placed superficial or deep to the GON and/or superficial or deep to the TON and/or at point equidistant from GON and TON, and/or at a location otherwise within therapeutically effective distance from both GON and TON, thereby activating targeted fibers in both the GON and TON to produce comfortable sensations in the innervation regions of both nerves and providing relief of pain that may occur in the innervation regions of both nerves and/or regions of referred pain. As another non-limiting example, one or more leads, electrodes, and/or contacts are placed lateral and superficial to the GON at the level of the C2 vertebra where both the GON and LON often traverse near one another, thereby activating targeted fibers in both the GON and LON to produce comfortable sensations in the innervation regions of both nerves and providing relief of pain that may occur in the innervation regions of both nerves and/or regions of referred pain. In additional non-limiting examples of embodiments of the present invention, the one or more leads, electrodes, and/or contacts may be placed targeting the GON and/or the TON and may also directly and/or indirectly activate, stimulate, excite, inhibit, block, and/or attenuate activity in the trigeminal nerve or a subset of fibers from the trigeminal nerve through interaction between the trigeminal nerve and the occipital nerves in the trigeminocervical complex, such as in cases of cervicogenic headache where pain may occur around and/or behind the eyes (e.g., retroorbitally) and top of the head (e.g., parietally) in the region innervated by the trigeminal nerve.

In another embodiment the lead is placed targeting the greater occipital nerve (GON), third occipital nerve (TON), and/or lesser occipital nerve (LON) at the level of the C2 lamina using an approach in the inferosuperior plane (e.g., inferior-to-superior or superior-to-inferior) approach, and in a preferred embodiment uses an inferior-to-superior approach. It is to be appreciated that this approach may include placement of the stimulating lead in a trajectory that is not exclusively in the inferior-to-superior direction (e.g., caudad to cephalad, or upward), and the lead may also and/or instead take an inferior-to-superior approach that also has an angle and/or component in the lateral or medial direction. In a non-limiting example of this embodiment, the lead is placed starting lateral to the midline directly inferior to the target location (e.g., 2-3 cm lateral to the midline, or over the lateral or articular pillar) and at least one spinal level and up to 4-5 spinal levels inferior (e.g., caudal or below) to the target location (e.g., at least at C3, or as far inferior as C7, T1, or more inferior levels) but not so far inferior such that the distance from the needle entry site to the target location of the stimulating electrode exceeds the total length of the introducer needle and/or lead. In a preferred non-limiting example of this embodiment, the lead is placed starting at a location approximately 4-5 cm inferior from the target location to ensure that a sufficient length of the coiled lead is placed into the body to enable healthy tissue ingrowth that increases the stability and security of the lead to overcome the challenges of lead migration and loss of effective stimulation due to the mobility of the tissues through which the lead is placed, and the lead is advanced superior at an appropriate angle such that the stimulating electrode is placed remote (e.g., 0.5-1 cm) from the GON at the level of the C2 lamina.

In one non-limiting example of this embodiment, fluoroscopic guidance is used such that the lead is placed using bony landmarks (e.g., cervical spinous processes, lamina, transverse processes, etc.) targeting the C2 lamina and redirecting the introducer needle superficial into the muscle overlying the C2 lamina and testing stimulation at locations along the insertion trajectory to determine the ideal lead location where the desired response is elicited from the target nerve (e.g., comfortable sensations covering the region of pain and/or the region of innervation of the target nerve, and/or pain relief in the area of innervation of the target nerve, and/or pain relief in one or more areas of referred pain outside the area of innervation of the target nerve). In another non-limiting example of this embodiment, ultrasound guidance is used such that the lead is placed remote (e.g., 0.5-1 cm) from the GON, such as above or superficial to the GON in the semispinalis capitis muscle, adjacent to the GON in the fascial plane between the semispinalis capitis muscle and obliquus capitis inferior muscle, or below the GON in the obliquus capitis inferior muscle, in a location where it is confirmed by testing the stimulation that the desired response is elicited from the target nerve (e.g., comfortable sensations covering the region of pain and/or the region of innervation of the target nerve, and/or pain relief in the area of innervation of the target nerve, and/or pain relief in one or more areas of referred pain outside the area of innervation of the target nerve). It is also to be appreciated that this approach may occur at the level of the C2 lamina, but may also occur above or below the level of the C2 lamina, such as at the C3 lamina, between the C2 and C3 lamina, at the C2/C3 facet joint, at the level of C1, etc., and an approach to the C3 lamina may be preferred, for example, if the target nerve is the TON and the region of pain occurs in the innervation region of the TON (e.g., the suboccipital region, or the region below the nuchal ridge or occipital prominence to approximately the bottom or caudal border of the mastoid process). In a non-limiting example, an additional lead may be placed targeting the lesser occipital nerve near the cervical plexus at the level of the C2 vertebra, where the LON may be targeted close to the GON and TON, such that the present system and method activate one or both the GON and TON in addition to the LON in cases where pain is in the distributions of multiple of these nerves.

In another version of this embodiment, the lead is placed targeting the greater occipital nerve (GON), third occipital nerve (TON), and/or lesser occipital nerve (LON) above the level of C2 and below the nuchal ridge (e.g., below the external occipital crest or median nuchal line that descends from the external occipital protuberance to the foramen magnum where the attachment of the nuchal ligament occurs). In one version of this embodiment, the introducer needle enters the skin on the ipsilateral side (e.g., the same side) of the head or neck as the target nerve(s), starting inferior or below the target location for the lead and advancing from inferior to superior under ultrasound and/or fluoroscopic guidance. In another version of this embodiment, the introducer needle enters the skin on the contralateral side (e.g., the opposite side) of the neck and advances at an angle from inferior to superior across the midline and proceeding from medial to lateral such that, if two leads are placed to stimulate the bilateral GON, the introducer needles and/or leads may cross each other (e.g., crisscross at or above the level of C1). In a non-limiting example, the leads are placed such that the stimulating electrode is remote (e.g., 0.5-1 cm) from the GON inferior to the nuchal ridge and stimulation of the GON produces coverage of comfortable stimulation-evoked sensations in the distribution of the GON (e.g., the back of the head).

In another embodiment, one or more lead(s) with one or more individual electrode contact(s) are placed targeting the lesser occipital nerve (LON) in a mediolateral approach inferior to where the lesser occipital nerve emerges from the sternocleidomastoid muscle and becomes superficial (e.g., subcutaneous, below the skin, and/or superficial to the muscle(s) under which it lay at more proximal locations). In a non-limiting example, the lead is placed ipsilateral (e.g., on the same side as the target LON) to the midline and inferior to the line connecting the left and right auditory canals such that the lead entry site is inferior and medial to the target nerve and insertion of the lead along a trajectory from the entry site laterally to a remote and/or therapeutically effective distance from the target nerve includes approximately 4-5 cm of lead implanted under the skin to facilitate health tissue ingrowth to secure the lead and prevent or avoid unwanted fracture, movement, migration, or other failures. For example, the lead may be placed lateral from the midline (e.g., 30 mm, 30-50 mm, ≥30 mm, >30 mm, ≥15 mm, >15 mm, 15-90 mm, 20-100 mm, 50-90 mm, and in one embodiment approximately 70 mm from the midline) and inferior to the line connecting the left and right external auditory canals (and in one embodiment approximately 55 mm (range: 30-80 mm)) such that the lead entry site is 4-5 cm medial to the target nerve but not farther from the target nerve than the length of the introducer needle, and implanting the lead along a trajectory from the entry site to the target nerve enables implantation of at least 4-5 cm of the lead. In another non-limiting example, a lead with multiple stimulating electrodes spaced along the lead target the LON using a mediolateral approach with the goal of placing one or more of the multiple electrodes within remote and/or therapeutically effective distance of the LON to generate comfortable sensations within the region of pain without generating muscle contractions. In another non-limiting example, the present invention overcomes the challenges of stimulating multiple target nerves (e.g., in this non-limiting example stimulating GON and LON) to produce pain relief in one or more regions of pain that are not limited to the distribution of the GON and/or LON and occur in both innervation regions and/or one or more referred pain regions by placing one lead targeting the LON in a mediolateral approach while a second lead is placed targeting the GON using an inferosuperior approach such that the leads are placed on non-intersecting trajectories in a case in which pain is present in the regions innervated by both the LON and GON.

The system may use multiple types of electrodes and leads. In an embodiment the lead(s) include a temporary or permanently implanted helically coiled, open-coil, or closed-coil lead design with one or more electrodes and/or contacts and enables use with a single-contact (e.g., single electrode) lead (e.g., a lead with only one electrode, enabling additional redundancy and reliability using more than one wire to connect to or provide conductivity to the electrode and/or contact). In other embodiments, the leads may include any other type of electrode, such as a straight or uncoiled fine wire, paddle electrode, needle electrode, cylindrical electrode, intramuscular electrode, fine-wire lead, fine-wire electrode, general-purpose electrode, skin surface, cutaneous, or any other appropriate type of electrode (whether known today or developed thereafter), placed or inserted via a needle introducer, percutaneously, non-invasively, and/or surgically implanted with one or more open incisions. In one embodiment, a coiled lead (e.g., a lead with multiple filars or filaments contained within or composing one or more strands or groups of filars or filaments), which may or may not use fine wires in its construction and may have one or multiple electrodes, is placed using a needle introducer and once proper placement of the lead and or electrode(s) (e.g., in its appropriate operative position) is confirmed (e.g., the electrode is placed remote from the target nerve at a therapeutically effective distance such that stimulation produces comfortable sensations focally in the region(s) of pain through the selective activation of target fibers in the nerve while avoiding, preventing, and/or prohibiting the activation of non-target fibers in the nerve, and while also being close enough to the nerve to activate target fibers in the nerve while avoiding, preventing, and/or prohibiting activation of non-target nerve fibers, nerves, structures, and/or tissues whose activation would produce discomfort, pain, motor activity, and/or or other undesired responses), the needle introducer may be withdrawn, leaving the lead(s) and/or electrode(s) in place. As non-limiting examples, a coiled lead may be comprised of one or more filars or filaments (e.g., 1, 2, 3, >=1, >1, 7, 12, 19, >19, ≥19, 50, 100, ≥100, >100 filars or filaments) contained within or composing one or more strands or groups of filars or filaments. Stimulation may also be applied through a penetrating electrode, such as an electrode array comprised of any number (e.g., one or more) of needle-like electrodes that may be inserted into the target site. In both cases, the lead are placed using a needle-like introducer, allowing the lead(s) and electrode placement to be minimally invasive. In a representative embodiment, the lead(s) include a thin, flexible component made of a metal and/or polymer material (e.g., ≤0.75 mm, 0.1-0.75 mm, 0.3-0.8 mm, ≤0.8 mm, ≤0.9 mm, 0.1-1.0 mm, 0.3-1.0 mm, 0.5-1.0 mm, <1.0 mm, ≤1.0 mm, ≤1.2 mm, and in a preferred embodiment the lead(s) may not be greater than about 0.75 mm (0.030 inch in diameter). However, the present teachings are not limited to such dimensions. Any appropriate electrode and lead may be utilized without departing from the present teachings.

In an embodiment the lead(s) include a temporary or permanently implanted helically coiled, open-coil, or closed-coil lead with dimensions (e.g., thin diameter) and mechanical attributes (e.g., flexibility and/or durability) that enable it to be compressed and/or flex when needed (e.g., to avoid damage to the tissue and/or the lead and to avoid, prevent, and/or prohibit erosion of hardware components (such as the lead(s), component(s) of the lead, electrode(s), contact(s), anchor(s), tine(s), lead body(ies), stimulator(s), receiver(s), connector(s), cable(s), and/or other components of the system) through one or more anatomical tissues, such as preventing erosion through, within, or to the skin and/or other tissues), but remains non-compressed and/or non-flexed when needed (e.g., to prevent unwanted movement or migration of the lead and/or electrode, such as preventing unwanted movement, migration(s), or motion in the axial or longitudinal direction or axis of the lead, and/or to prevent non-repetitive or repetitive (e.g., cyclical) stress, strain, shear, or other loading of the lead that weakens and/or otherwise contributes to or causes breakage, fracture, or loss of electrical continuity of the lead instantly or over time (e.g., through accumulation of mechanical forces or repetitions of cyclical loading)).

The lead(s) may also include one or more coiled metal wires with an open or flexible elastomer core. The wire may be insulated, e.g., with a biocompatible polymer film, such as polyfluorocarbon, polyimide, or parylene, or other appropriate insulating material. The lead may be electrically insulated everywhere except at, for example, one (monopolar), or two (bipolar), or three (tripolar), or four (quadripolar) or more than four (e.g., 5, 6, 8, 10, 12, 16, 32) conduction locations near its distal tip of the same or different sizes, lengths, and/or dimensions (e.g., 0.1 mm, 0.2 mm, 0.3 mm, 0.4 mm, 0.5 mm, 1 mm, 3 mm, 4 mm, 5 mm, 7 mm, 10 mm, 15 mm, 20 mm, 1.0 cm, 0.2-1.5 cm, 0.1-3.0 cm, 0.1-4.0 cm, and/or 1-10 cm in length) separated by non-conducting lengths of the same or different sizes and/or dimensions (e.g., 0.1 mm, 0.2 mm, 0.3 mm, 0.4 mm, 0.5 mm, 1 mm, 3 mm, 4 mm, 5 mm, 7 mm, 10 mm, 15 mm, 20 mm, 1.0 cm, 0.2-1.5 cm, 0.1-3.0 cm, 0.1-4.0 cm, and/or 1-10 cm in length). Each of the conduction locations may be connected to one or more conductors that may run the length of the lead(s) and extension cable or cables used to connect the lead to an external or internal pulse generator/stimulator or a portion thereof. The conductor may provide electrical continuity from the conduction location through the lead to an external or internal pulse generator or stimulator.

In one embodiment, the one or more conduction locations are located and/or concentrated at the distal tip of the electrode such that the tip of the introducer needle coincides with the implanted location of the distal tip of the lead and therefore with the location of the electrode contact(s). In another embodiment, the one or more conduction locations are arrayed, lined up, patterned, and/or arranged along the length of the lead such that one contact is located at the distal tip of the lead and one or more additional contacts are located proximal to the distal tip, closer to the external pulse generator (EPG), internal pulse generator (IPG), or skin exit site relative to the first contact and the distal tip of the lead. In another embodiment, the one or more conduction locations are proximal to the distal tip of the electrode such that the one or more electrode contacts are all closer to the EPG, IPG, or skin exit site relative to the distal tip of the electrode and the distal tip of the electrode does not include an electrode contact. The one or more electrode contacts may be individually selectable to deliver monopolar stimulation, individually selectable or assignable in any combination of adjacent and/or non-adjacent electrodes in bipolar, tripolar, quadripolar, or other configurations, and/or electrically coupled in any combination of adjacent and/or non-adjacent electrodes to deliver stimulation by simulating, creating, and/or resembling a single, larger monopolar electrode.

The present system and method, in one embodiment, include a lead that is an insulated coiled metal wire with an open core and one deinsulated or uninsulated segment that delivers electrical stimulation to the tissue (e.g., an electrode contact) located a distance (e.g., 0.1 cm, 0.2 cm, 0.3 cm, 0.4 cm, 0.5 cm, 1 cm, 1.2 cm, 1.5 cm, 2 cm, 3 cm, 4-5 cm, >1 cm, >3 cm, etc.) proximal on the lead. As a non-limiting example a 1 cm electrode contact located 3 cm proximal from the distal tip of the lead would occur from the 3^{rd} to 4^{th} cm of the lead when measuring from the distal tip of the lead while first 3 cm of the lead are insulated and the 4th cm through the proximal end of the lead are also insulated. Whereas previous leads that have a single, monopolar electrode contact at the distal end of the lead require the distal end of the lead to be within a remote stimulating distance (e.g., 0.5-3 cm) from the target nerve for therapeutic electrical stimulation of the nerve while also achieving a minimum length (e.g., >3-4 cm) of lead implanted to facilitate healthy tissue ingrowth and reduce the risk of migration or dislodgement or infection, in this embodiment of the present system and method the lead are inserted in any acceptable trajectory such that the distal lead tip is located far from the target nerve (e.g., >0.5 cm, >3 cm, >4 cm) and the electrode contact is positioned within a remote stimulating distance from the nerve to enable targeting of nerves for which anatomical limitations prevent a lead entry site far from the nerve and the lead entry site is necessarily close to the nerve. As a non-limiting example, the third occipital nerve at the level of the C2 lamina is typically approximately 1 cm lateral from the midline such that placing the lead with an introducer needle entering the skin at the midline cannot achieve 3-4 cm of lead implanted under the skin while maintaining the distal tip of the lead within remote stimulating distance of the target nerve. Alternate approaches are also challenging with other leads whose electrode contact is at the distal tip of the lead, for example the spinous processes can block needle passage making a contralateral entry difficult, and lateral to medial approaches may be difficult due to the mastoid process and lateral curvature of the neck. In the present non-limiting example, the lead is introduced at the midline and advanced at least 3-4 cm laterally to achieve the desired length of lead implanted under the skin to facilitate healthy tissue ingrowth and reduce the risk of migration or dislodgement or infection, while also enabling the electrode contact to be placed over, under, and/or near the target nerve within remote stimulating distance.

The lead may also include an anchoring element, at its distal tip and/or at one or more locations proximal (closer to the EPG, IPG, and/or skin exit site) to one or more electrode contacts (e.g., behind or closer to the EPG, IPG, and/or skin exit site relative to the first electrode contact, behind or closer to the EPG, IPG, and/or skin exit site relative to a second electrode contact, behind or closer to the EPG, IPG, and/or skin exit site relative to one, more than one, or all electrode contacts, etc.). In the illustrated embodiments, the anchoring element may take the form of a simple or complex anchor, barb, tine, and/or bend and/or collection of multiple or groups of anchors, barbs, tines, and/or bends. The anchoring element(s) may be sized and configured during or after manufacturing so that, when in contact and/or anchoring in, with, and/or within tissue, it (or they) take(s) purchase in tissue, to resist dislodgement or migration of the electrode out of the correct location in the surrounding tissue. Desirably, the anchoring element(s) may be prevented from anchoring, engaging, and/or fully engaging body tissue until after the electrode has been correctly positioned, located and deployed, and the anchor(s) may be designed to flex, bend, compress, stress, and/or otherwise change shape, contour, thickness, and/or profile, to avoid damaging tissue and/or the lead or anchor itself during removal. However, it is to be appreciated that the anchor(s) may also be designed to be rigid or sufficiently rigid such that they do not bend or flex to increase the securement of the lead in the tissue such as, in a non-limiting example, a long-term or permanently implanted lead that is not intended to be easily removed unintentionally (e.g., not intended to be easily displaced or moved when not desired) but can be easily removed when desired (e.g., on purpose by the physician, clinician, and/or patient user once the patient's condition (e.g., pain, disability, medication dependence, etc.) has been adequately or sufficiently treated and/or resolved due to or following use of the invention.

In an embodiment, the lead(s) may exit through the skin and connect with one or more external electrical stimulation devices using cabling, connectors, and/or other components, features, and specifications. Further, the lead(s) may be connected electrically and/or mechanically as needed to internal and/or external coils for RF (Radio Frequency), inductive coupling, and/or wireless powering and/or telemetry communications or an inductively coupled telemetry to control the stimulation device(s).

The lead(s) may also be fully implanted and electrical stimulation may be delivered with a temporary or permanent fully implantable system, which could include a fully implantable lead and an implantable pulse generator (IPG) which may be controlled by external devices (such as a patient and/or clinician programmer and/or controller). The IPG may be powered by a(n) internal source(s), such as a rechargeable battery, energy banking, a primary cell or non-rechargeable battery, or other means. The implantable system may also be powered by external sources, including an external pulse transmitter, radiofrequency (RF) powering, induction, or other means. As a non-limiting example, the present systems and methods may be designed, in an embodiment, to overcome the challenges of previous systems and methods (e.g., high rates of adverse events, including pain and discomfort at the site of the IPG due to the subcutaneous implantation of a large IPG (e.g., volumes ranging from 14-40 cc), as well as lead fracture, migration, or other failure associated with tunneling of leads from the occipital nerves in the upper neck or posterior head down the length of the back/torso to the location of the IPG in the upper buttocks or lower abdomen, subjecting the leads to multiple points of stress, strain, shear, and/or other mechanical loading as they traverse joints, planes, axes of movement, muscle, fascial, and other tissue layers, and are subjected to twisting, bending, and other sources of strain and shearing during back and neck movements) by eliminating the need to tunnel leads long distances through the use of an IPG (e.g., a miniaturized IPG whose volume is, for example, <40 cc, <20 cc, <14 cc, <10 cc, <7 cc, <5 cc) whose dimensions, volume, and/or size are suitable for placement near the site of stimulation of the occipital nerves such as, in non-limiting examples, the posterior neck, upper back, or infraclavicular region, eliminating the need for leads to cross multiple joints, muscles, muscle planes, tissues, tissue planes, axes of movement, bending, or twisting, and minimizing tunneling distance to reduce the repeated bending, movement, stress, strain, shearing, and/or mechanical loading of the leads.

Control of the electrical stimulation device (e.g., the pulse generator) and its stimulation parameters may be provided by one or more external controllers. Alternatively, a controller may be integrated with the external electrical stimulation device. The implanted pulse generator external controller (e.g., clinical programmer) may be a remote unit that uses RF (Radio Frequency) wireless telemetry communications (rather than an inductively coupled telemetry) to control the pulse generator. The electrical stimulation device may use passive charge recovery to generate the stimulation waveform, regulated voltage (e.g., 10 mV to 20 V), and/or regulated current (e.g., about 10 mA to about 50 mA). Passive charge recovery may be one method of generating a biphasic, charge-balanced pulse as desired for tissue stimulation without severe side effects due to a DC component of the current.

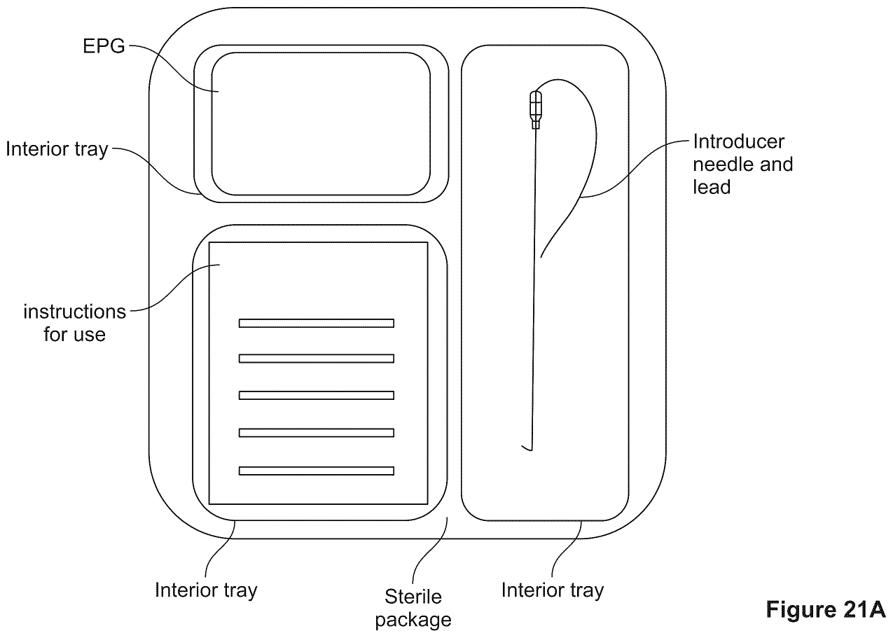

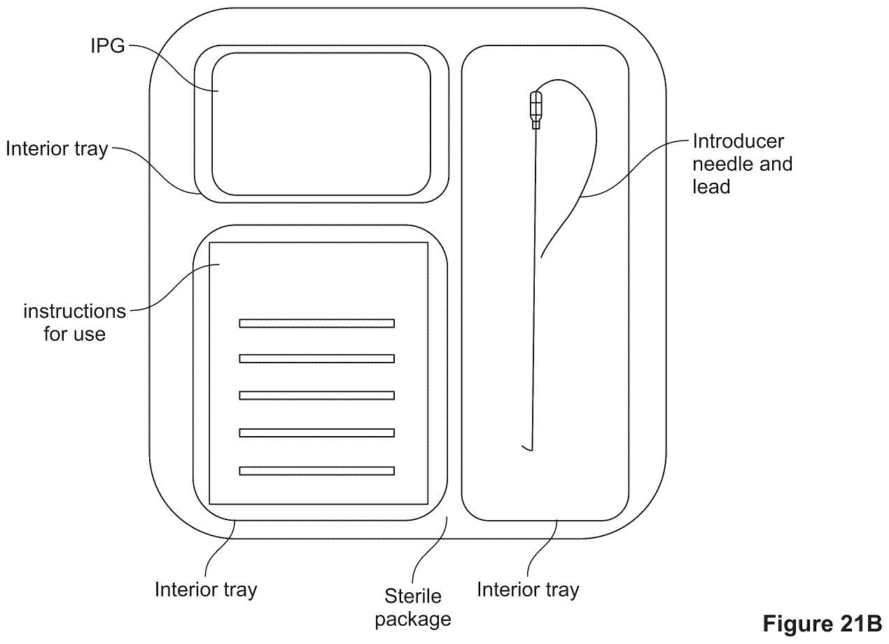

The lead(s) may be provided in a sterile package and may be pre-loaded in an introducer needle. Alternatively, the lead may be introduced via the same needle that is used to inject anesthetic or analgesics during peripheral nerve blocks, which are a common interventional procedure for chronic pain patients. The sterile package may take various forms and the arrangement and contents of the sterile package may be as appropriate related to the use thereof. The sterile package may include a sterile, wrapped assembly. The sterile package may include an interior tray made from any appropriate material, e.g., from die cut cardboard, plastic sheet, or thermos-formed plastic material, which may hold the contents. The sterile package may include any appropriate number of interior trays, including, without limitation, one, two, three, four, etc., including as a non-limiting example the three such interior trays shown (FIG. 21A). The sterile package may also desirably include instructions for use regarding using the contents of the sterile package to carry out the lead(s) location and placement procedures.

Inserting the lead(s) percutaneously allows the lead(s) to be placed quickly and easily. In one embodiment, the lead(s) may be placed percutaneously via an introducer needle to place the lead(s) targeting one or more peripheral nerves without the use of incisions, surgical dissection, or open surgical techniques in less time and without requiring regional or general anesthesia, enabling subjects to provide verbal feedback and/or confirmation of the location of stimulation-evoked sensations such that the lead(s) may be optimally placed to generate comfortable sensations in the region of pain. The introducer needle is made from conductive and/or non-conductive materials, with the stimulating portion of the lead (e.g., the electrode) housed inside. One or more electrode contacts on the lead may be housed inside the introducer needle, and/or one or more electrode contacts or the stimulating portion of the lead may protrude from the end of the needle itself so as to come into contact with the body tissue in which the lead is inserted. The distal end of the electrode may also protrude from the end of the needle in the same manner. In one embodiment, stimulation is delivered through the tip of the needle and echogenic markings determine how far a lead with electrodes proximal to the distal tip would need to be advanced to match the stimulation of the needle.

In another embodiment, stimulation is delivered through the tip of the needle and markings, measurements, guides, needle stops, and or other visual or mechanical aids on the external portion of the introducer (e.g., outside the body) determine how far a lead with electrodes proximal to the distal tip would need to be advanced to match the stimulation pattern delivered from the tip of the needle and/or to place the electrode contact in the location identified by stimulation from the tip of the needle. In another embodiment, the introducer may have one or more deinsulated portions that mimic the conductivity pattern of the electrode contacts on the lead and electricity may be delivered through the deinsulated portions of the introducer during the procedure to aid in determining the optimal location for lead placement while mimicking the stimulation pattern that will be delivered by the electrode after deployment from the introducer needle. In this embodiment the electrode contacts of the lead inside the introducer may conduct through direct contact on the inside surface of the introducer needle to the deinsulated portions of the introducer to produce stimulation during the procedure and/or the introducer needle may be connected directly to the electrical stimulation device. The needle, lead, and/or electrode are then connected to an electrical stimulation device, such as an external pulse generator during/as a part of the introducer/implantation process. Applying stimulating current through the electrode while it is housed within the introducer may provide a close approximation to the response that the electrode will provide when it is deployed at the location of the introducer needle because stimulation will be delivered through the same conductive portion of the electrode.

Pulses may be applied in continuous or intermittent trains (e.g., the stimulus frequency changes as a function of time). In the case of intermittent pulses, the on/off duty cycle of pulses may be symmetrical or asymmetrical, and the duty cycle may be regular and repeatable from one intermittent burst to the next or the duty cycle of each set of bursts may vary in a random (or pseudo random) fashion. Varying the stimulus frequency and/or duty cycle may assist in warding off habituation because of the stimulus modulation. As a non-limiting example, the stimulation train may consist of periods (e.g., 10 s, 30 s, 5 min, 60 min, 6 h, 12 h, and/or 24 h) of stimulation at a constant frequency (e.g., 0.1-150, 1-150, 0.1-200, 0.1-1499, 0.1-1500, 0.1-20000, 1500-20000, >150, ≥150, >1499, >1500, ≥1500, 12, 20, 30, 40, 50, 60, 70, 80, 90, 100, 150, 200, 500, 1000, 1499, 1500, 2000, 5000, 10000, and/or 20000 Hz) followed by periods of inactivity that may be the same duration or a different duration as the period of stimulation such that the duty cycle is less than 100% (e.g., a 10% duty cycle whereby the inactive period is 9× the length of the active period, a 50% duty cycle whereby the inactive period is equal in duration to the active period, 75% duty cycle whereby the inactive period is one third the duration of the active period, etc.). The stimulation train may also include a gradual ramp up (e.g., each successive stimulation pulse has greater intensity, beginning at zero and increasing to the desired therapeutic stimulation intensity over the course of a period (e.g., 1-60 s, 0.1-10 min, 5-60 min)) in intensity at the transition between the inactive and active periods and a gradual ramp down (e.g., each successive stimulation pulse has lower intensity, beginning at the therapeutic stimulation intensity and decreasing to zero over the course of a period of time (e.g., 1-60 s, 0.1-10 min, 5-60 min)) in intensity at the transition between the active and inactive periods.

The stimulating frequency may be selected from a range of frequencies (e.g., 1-100 Hz, 1-300 Hz, 1-1000 Hz, 1-1200 Hz, 1-1499 Hz, 1-1500 Hz, 1-10000 Hz, 1-20000 Hz, 1-100000 Hz, 5-100 Hz, 5-150 Hz, 5-1200 Hz, 5-1500 Hz, 12-100 Hz, 12-150 Hz, 12-1500 Hz, 1200-10000 Hz, 1200-20000 Hz, 1500-10000 Hz, 1500-100000 Hz, and/or 10,000-100,000 Hz). The frequency of stimulation may be constant or varying. In the case of applying stimulation with varying frequencies, the frequencies may vary in a consistent and repeatable pattern or in a random (or pseudo random) fashion or a combination of repeatable and random patterns.

In an embodiment, the stimulator may be set within a range of intensities including amplitude (e.g., 0.01-1 mA, 0.1-2 mA, 0.1-5 mA, 0.1-30 mA, 0.1-50 mA, 1-3 mA, 1-5 mA, 1-20 mA, 1-30 mA, 1-50 mA) and pulse width (e.g., 0.01-1 μs, 0.01-3 μs, 0.1-5 μs, 0.1-10 μs, 0.1-50 μs, 1-150 μs, 1-1000 μs, 1-2000 μs, 50-200 μs, 10-2000 μs), and in a preferred embodiment any combination of two whole integers or non-integers between 1.0 and 30.0 mA and, separately, between 10 and 200 microseconds, so as to form lower and upper limits for each.

If the stimulus intensity is too great, it may generate muscle twitch(es) or contraction(s) sufficient to disrupt correct placement of the lead. If stimulus intensity is too low, the lead may be advanced too close to the target peripheral nerve (beyond the optimal position), possibly leading to incorrect guidance, mechanically evoked sensation (e.g., pain and/or paresthesia) and/or muscle contraction (e.g. when the lead touches the peripheral nerve), inability to activate the target nerve fiber(s) without activating non-target nerve fiber(s), improper placement, and/or improper anchoring of the lead (e.g., the lead may be too close to the nerve and no longer able to anchor appropriately in the muscle tissue).

The lead may be designed for or capable of permanent use but used on a non-permanent or temporary basis or use case. The lead may be a permanent or temporary, removable, short-term, and/or non-permanently implanted percutaneous lead that is flexible, coiled, migration-resistant, fracture-resistant, infection-resistant, self-anchoring (e.g., stabilized by an integrally formed component or portion of the lead, electrode, and/or anchoring element such that additional components, parts, or anchoring techniques are not needed to avoid lead migration and/or dislodgement) and/or anchored by one or more non-integrally formed, additional, external, internal, supplementary, and/or anchoring, fixation, fixing components or techniques, wherein the coiled structure may enable the lead to flex and bend when subjected to forces rather than migrate or fracture. The lead(s) may possess mechanical properties in terms of flexibility and fatigue life that provide an operating life free of mechanical and/or electrical failure, taking into account the dynamics of the surrounding tissue (e.g., stretching, bending, pushing, pulling, crushing, etc.). There may be one or more coils, with the coils composed of bundles of conductive wires (e.g., a plurality of wires, filars, filaments, and/or strands of wire(s) formed into one or more conductive bundle(s) which may or may not be electrically isolated from each other or from the tissue). Each of the conductive wires, filars, filaments, and/or strands, as well as the coils, may be connected or wired in parallel to the electrical stimulation/pulse generator and/or electrode or contact to ensure that a fracture sustained in a single wire, filar, filament, and/or strand does not inhibit, interrupt, prevent, or prohibit stimulation or therapeutic pulses delivered by the lead as a whole. Similarly, each of the conductive wires, wires, filars, filaments, and/or strands, as well as the coils, may be connected or wired in parallel to the electrical stimulation/pulse generator and/or electrode or contact to ensure that a fracture sustained in a one or more wires, filars, filaments, and/or strands do(es) not inhibit, interrupt, prevent, or prohibit stimulation or therapeutic pulses delivered by the lead as a whole. In this embodiment, the helical or open-coil structure of the lead is desirable such that the interstices of the coils will enable, facilitate, and/or cause tissue ingrowth to better secure the lead within the patient's body, thereby reducing lead migration. However, it is to be appreciated that it may be desirable to discourage the in-growth and/or limit the extent of the in-growth of connective tissue along its length or an applicable portion thereof, so as not to inhibit its withdrawal at the end of its use, for example through the use of a closed coil or non-coiled lead and/or the use of materials that discourage tissue ingrowth.

In one embodiment, the system may be a short-term and/or temporary system (e.g., the system is not intended to be permanent and/or indefinite) wherein the temporary and/or short-term treatment period may range from minutes to hours to days to weeks to months. By way of a non-limiting example, the short-term and/or temporary treatment period may be between approximately 21 and 30 days, or between approximately 56 and 60 days or between 21 and 60 days or longer (e.g., up to 60 days, up to 90 days, 90-120 days, 3-6 months, 3-12 months, 1-3 years, 1-5 years, 2-5 years, 1-10 years, etc.), while in another embodiment the system may be a permanently implanted system intended to provide the patient with therapy indefinitely. In the non-limiting example of a short-term and/or temporary system, it may be advantageous to avoid permanent implantation of lead(s), pulse generator(s), and/or other system components because patients may achieve long-term sustained pain relief from a short-term treatment, obviating the need for permanent implantation and reducing the safety risks and financial costs associated with permanent implantation. In this embodiment one or more temporary, short-term, and/or removable lead(s) may be inserted percutaneously and electrical coupled (e.g., directly and/or using one or more connectors, couplers, cables, or other components or features) to an external pulse generator and may deliver stimulation for a temporary or short-term treatment period through the one or more leads independently (e.g., on one or more separate, independent stimulation channels or programs) or non-independently (e.g., wherein the one or more leads are electrically coupled and deliver the same stimulus or program). In embodiments of this percutaneous system, one or more surface electrode(s) may serve as the anode(s) (or return electrode(s)). The surface electrodes may be a standard shape or they may be modified as appropriate to fit the contour of the skin. When serving as a return electrode(s), the location of the electrode(s) may not be critical and may be positioned anywhere in the general vicinity (e.g., on the shoulder, abdomen, lower back, or upper or lower extremity), provided that the current path does not cross parts of the body (e.g., the heart), through which stimulation could be harmful.

In another embodiment, the system may be a long-term and/or permanently implanted system wherein the treatment period may be continuous or indefinite (e.g., the system is not intended to be removed) and individual stimulation sessions may be applied that range from minutes to hours to days to weeks to months to continuous or indefinite but the implanted lead and other system components are not removed or explanted in between individual stimulation sessions. By way of a non-limiting example, the system may deliver stimulation continuously for many years and/or indefinitely, or the system may deliver stimulation for any finite period or session consisting of seconds, minutes, or hours per day, or days per week, or weeks per month, or months per year in a repeating cycle (e.g., 30 minutes per day, 60 minutes per day, 3 hours per day, 6 hours per day, 12 hours per day, 2 days per week, 4 days per week, 5 days per week, 1 week per month, 3 weeks per month, 1 month per year, 4 months per year, 6 months per year, etc.). In this embodiment, it may be advantageous to permanently implant lead(s), pulse generator(s), and/or other system components to provide patients with long-term sustained pain relief through the continuous and/or intermittent and/or on-demand delivery of stimulation in situations where short-term stimulation has not, does not or is unlikely to produce sustained relief. The lead may be an implanted, or long-term, or permanently implanted lead that is flexible, self-anchoring, migration resistant, fracture resistant, and/or infection resistant, and may have an open coil, closed coil, or non-coiled structure that enables the lead to flex and bend when subjected to forces rather than migrate or fracture. The lead(s) may possess mechanical properties in terms of flexibility and fatigue life that provide an operating life free of mechanical and/or electrical failure, taking into account the dynamics of the surrounding tissue (e.g., stretching, bending, pushing, pulling, crushing, etc.). There may be one or more open or closed coils, with the coils composed of bundles of conductive wires (e.g., a plurality of strands of wire formed into a conductive bundle). Each of the conductive wires, as well as the coils, are wired in parallel to the electrical stimulation/pulse generator to ensure that a fracture sustained in a single wire does not inhibit therapeutic pulses delivered by the lead as a whole.

In this embodiment, one or more implanted, long-term, and/or permanently implanted lead(s) may be inserted percutaneously and electrically coupled (e.g., directly and/or using one or more connectors, couplers, cables, or other components or features) to an implanted internal pulse generator (IPG) and may deliver stimulation for a long-term and/or indefinite treatment period through the one or more leads or electrodes independently (e.g., on one or more separate, independent stimulation electrodes, contacts, channels or programs) or non-independently (e.g., wherein the one or more leads and/or one or more electrodes or contacts are electrically coupled and deliver the same stimulus or program). In embodiments of this permanently implanted system, one or more electrodes or contacts on a lead and/or the IPG (i.e., the “case”) may serve as the anode(s) (or return electrode(s)). When serving as a return electrode(s), the location of the IPG may not be critical and may be positioned anywhere in the general vicinity (e.g., on the shoulder, abdomen, lower back, or upper or lower extremity), provided that the current path does not cross parts of the body (e.g., the heart), through which stimulation could be harmful.

Patients may undergo implantation of a temporary system for a short-term, temporary stimulation treatment, and/or may undergo implantation of a permanent system for a long-term and/or indefinite stimulation treatment in lieu of and/or following implantation of a temporary system for short-term treatment. It may also be advantageous for patients to receive a short-term, temporary stimulation treatment as a short-term trial prior to implantation of a permanent stimulation system, wherein the short-term system delivers the same stimulation therapy as the permanent system and may provide the physician and/or patient with information, data, and/or predictive knowledge about the likelihood of achieving successful outcomes (e.g., reduced pain, increased function, reduced disability, etc.) from a permanently implanted system. In this way, a temporary system may be implanted to provide short-term stimulation treatment and provide long-term, sustained, and/or definitive pain relief, and/or may be implanted to provide temporary, transient, or short-lasting pain relief while validating, confirming, and/or otherwise indicating that implantation of a permanent system for a long-term stimulation treatment is needed and/or would be beneficial for a given patient to provide long-term, sustained, and/or definitive pain relief. The short-term system may preferably deliver the same stimulation therapy (e.g., deliver the same stimulation waveform and/or parameters; utilize a temporary, short-term percutaneous lead with similar electrode size, surface area, and/or other properties or dimensions as the permanently implanted lead). However, it should be recognized that the short-term percutaneous system may not necessarily or preferably deliver the same stimulation waveform and/or parameters as the permanently implanted system, and/or other components, properties, dimensions, specifications, or features of the short-term and permanently implanted systems may not be identical or similar.

Completing a short-term trial with a temporary, non-permanently implanted system may be advantageous prior to implantation of a permanent system because it may reveal patients who are not likely to be responders to stimulation (e.g., not likely to achieve satisfactory pain relief or other benefits from stimulation) and therefore can avoid the risks and costs of permanent implantation. A short-term trial that is extended (e.g., 60 days) compared to prior conventional system trials that are commonly 7-10 days in length can additionally identify patients who are delayed responders and may have otherwise been ruled out of consideration for implantation of a permanent system due to a poor initial response in the first 7-10 days that later increases to provide patients with significant pain relief. A short-term trial that is extended (e.g., 60 days) compared to prior conventional system trials that are commonly 7-10 days in length can additionally identify patients who are delayed non-responders and may have otherwise been implanted with a permanent system due to a strong initial response in the first 7-10 days that later wanes due to, for example, accommodation, habituation, or loss of placebo effects, and require surgical revision and/or explanation of the permanent system.

As an example of a challenge the present system overcomes that distinguishes it from the prior art, previous approaches have sought to relieve pain in the distribution of the targeted nerve, whereas the present system is intended to relieve pain in the region innervated by the targeted occipital nerves as well as one or more regions of referred pain that occur outside the innervation region of the occipital nerves.

As another example of a challenge the present system overcomes that distinguishes it from the prior art, previous approaches have placed leads over the back of the skull and/or between the scalp and occipital bone, placing the leads in close proximity to non-target nerve fibers (e.g., pain fibers) in the cutaneous and subcutaneous tissues, whereas the present system places leads inferior to the nuchal ridge and/or inferior to C1 where there is greater depth of tissue within which to place the leads where the electrical stimulation does not activate non-target fibers in the cutaneous and subcutaneous tissues.

As another example of a challenge the present system overcomes that distinguishes it from the prior art, while placing the leads below the nuchal ridge enables stimulation that avoids the activation of non-target fibers in the skin, the area below the nuchal ridge is more mobile during physiological movement of the head and neck and this region introduces challenges of lead migration, fracture, and erosion of the lead through tissue such as to and/or through the skin. The present system overcomes these challenges through the use of a lead that is durable enough to withstand the forces placed upon the lead by movement of the head and neck, while also thin and flexible enough to bend and flex with the tissue rather than eroding through the tissue, while also anchored securely by healthy tissue ingrowth and/or anchoring structures to bend and flex with the tissue while avoiding migration of the electrode outside of the optimal location relative to the nerve. The combination of durability, flexibility, and secure anchoring enable a lead to be placed below the nuchal ridge and maintain an electrode within a therapeutically effective distance from the occipital nerve throughout a therapy period. The lead of the present system may comprise a coiled lead formed through wires wrapped in a defined manner to provide this strength and flexibility to achieve the advantages identified above.

As another example of a challenge the present system overcomes that distinguishes it from the prior art, while fracture and migration are common challenges for placement of electrical stimulation leads throughout the body, the area around the occipital nerves has unique challenges in that the space around the nerves is limited (e.g., by the presence of the vertebra on the medial and anterior/deep sides of the nerves), the head and neck are highly mobile and place substantial mechanical forces on the leads, and the target occipital nerves are bounded by densely arranged non-target structures including cutaneous and subcutaneous tissues as well as a series of small, complexly innervated muscles responsible for various movements of the head and neck. The present system includes a combination of a lead that can overcome the risk of migration and fracture while avoiding erosion, an electrode placed a therapeutically effective distance away from the nerve, and stimulation parameters that collectively enable activation of target fibers avoiding activation of non-target fibers in the multiple cutaneous, subcutaneous, and muscle structures closely surrounding the occipital nerves.

Representative lead insertion techniques will now be described to place an electrode and lead in a desired location in tissue in electrical proximity to but spaced away from a peripheral nerve such as the occipital nerves for the treatment of head and/and neck pain and/or headache. The procedures for application of the system and method to relieve chronic headache and/or head or neck pain in individuals may include any combination of the following: Screening, Baseline, Lead Placement Testing (or verification), Home Use, Lead Removal, and/or Follow-up.

Screening: Adults who have suffered head and/or neck injury resulting in chronic headache or who otherwise experience head or neck pain associated with the occipital nerve(s), such as cervicogenic headache and occipital neuralgia, are prime candidates. Other candidates may include those with chronic headache or other types of head and neck pain, especially primarily or partially in the distribution of the occipital nerve(s) and/or regions of referred pain associated with and/or communicating with and/or convergent with the occipital nerves (e.g., the trigeminal nerve(s)). As a non-limiting example, it is common for those with past mild traumatic brain injury (TBI) to suffer from headache pain in the occipital and suboccipital regions innervated, respectively, by the GON and TON. As an additional non-limiting example, impingement of the occipital nerve(s) can cause pain in the region innervated by the nerve and referred pain around the eyes (e.g., retroorbitally) and on the top of the head (e.g., parietally).

The present teachings may employ comparative or diagnostic blocks to optimize patient selection and to verify the targeted region of the occipital nerve(s) that should be innervated for pain relief. Variability may be reduced by using comparative or diagnostic blocks, where the effect of an anesthetic block is evaluated and/or compared to the effect of one or more subsequent blocks with a different anesthetic agent or saline (placebo) to rule out potential placebo-responders. When pain is confirmed to be in the distribution of the peripheral nerve receiving stimulation (e.g., occipital nerve), successful pain relief can be achieved in over 80% of patients. This verification may also be accomplished by way of an electrode delivered via a needle probe, via the stimulating lead or other means of testing and, preferably, comparing pain response to definitively identify the targeted area.

Patients may be screened using diagnostic or comparative block of the occipital nerves to confirm that their pain is in the regions innervated by the nerves and/or to help determine the optimal nerve target(s) for stimulation (e.g., one or more of the GON, TON, and/or LON). Ultrasound-guided injections may be applied to the occipital nerves using standard procedures. Ultrasound guidance enables more specific targeting of the occipital nerves and reduces the chance of false-positive responses from block of other nerves. For example, patients may receive either actual block or sham block at one time point and receive the other block at another time point. The order of presentation can be randomized, and both the patient and evaluator can be blinded to further improve efficacy. Actual blocks may consist of 2.5 cc of 1% lidocaine, and the sham injection may include 2.5 cc of saline. The half-life of lidocaine is 1.5-2 hours, and the effects of the block will have dissipated sufficiently by subsequent visits (>24 hours later). Alternatively, neurostimulation or other methods may be used in this step.

For a comparative block, it may be desirable for the screening to produce some (e.g., ≥30%, >50%, ≥50%, >70%, ≥70%, >80%, ≥80%, etc.) reduction in pain lasting for some duration (e.g., >10 minutes, ≥10 minutes, >20 minutes, ≥20 minutes, >30 minutes, ≥30 minutes, >45 minutes, ≥45 minutes, >60 minutes, ≥60 minutes, etc.) to demonstrate that the patient's pain is localized to the regions innervated by the occipital nerve(s), and it may be desirable for any placebo (sham) injection not to produce pain reduction (e.g., not greater than 30%) to confirm that that the responses observed with the actual injections were not due to a placebo response. In addition to confirming that the region of pain is innervated by the targeted occipital nerve(s), this comparative placebo-controlled block may also reduce the proportion of placebo responders who may not otherwise sustain long term benefits according to this invention. For a diagnostic block, it may be desirable for the region of pain to occur primarily (e.g., ≥30%, >30%, ≥40%, >40%, >50%, ≥50%, >70%, ≥70%, >75%, ≥75%, >80%, ≥80%, at least 90%, up to 100%, etc.) In the distribution of the effects of the block to verify that the region of pain is in the innervation region of the occipital nerves.

Baseline: After qualifying, patients may record outcome measures, such as pain, disability, physical function, medication usage and pain characteristics (headache duration, and frequency) for a period of time, preferably at least one week. The record should be started at least 24 hours after screening to ensure that the effects of the comparative or diagnostic block have completely disappeared or dissipated.

Lead Placement Testing: Patients will be prepared for the outpatient lead placement procedure. Hair on the back of the head may be removed, cleansed, and draped using aseptic technique. Alternatively, the hair may remain, and the lead may be inserted below the hairline and directed towards the occipital nerve (e.g., superiorly, towards the top of the head). Local anesthesia may be administered at the insertion site prior to lead implantation. If necessary, intravenous (IV) conscious sedation may be administered prior to lead placement. Lead placement may be guided using imaging, such as ultrasound imaging and/or fluoroscopy or any other appropriate method.

Leads will be placed to target the occipital nerves and/or as further determined by the screening. If the patient's pain is unilateral as described by the patient and confirmed via the nerve blocks used during screening, then one or more leads may be placed to target the appropriate occipital nerves on the side of pain. In a non-limiting example, a patient may have one lead placed targeting the greater occipital nerve and another targeting the lesser occipital nerve unilaterally for pain in the regions innervated by both nerves. Leads may also be placed to target the occipital nerves on each side of the head for bilateral pain.

Test Needles or Stimulating Probes may be used prior to placement of the temporary percutaneous lead. The Test Needle or Stimulating Probe may be advanced and retracted until the optimal location is determined to guide the subsequent placement of the percutaneous leads. The percutaneous leads may be placed within tissue proximal to the nerves (preferably, approximately 0.5 cm away from nerve). In all cases, the lead need not come into direct contact with the nerve.

During Lead Placement, a verification step may be performed by delivering test stimulation to provide comfortable sensations to the regions of pain, verifying that electrode placement and stimulus intensity are sufficient to activate the target nerve and provide pain relief and/or comfortable sensations in the region of pain.

During lead placement, if pain is present in the regions innervated by the lesser and/or third occipital nerves, then these nerves may be targeted as an additional or alternative treatment approach.

Home Use: Patients will use the system without the need for supervised care. Because of the mobile nature of the system and resistance to negative sequelae of movements, the patient should be able to continue with daily activities. Stimulation may be delivered continuously for up to 24 hours a day. In another non-limiting example, stimulation may be delivered for a set number of hours per day (e.g., 6 hours per day). In another non-limiting example, stimulation is delivered as needed (e.g., only when experiencing a headache, or when the patient experiences a migraine aura). Stimulation may be used to treat a headache in progress and/or to prevent headaches (e.g., prophylactically). In another non-limiting example, stimulation may be delivered using non-continuous trains of pulses (e.g., duty cycle), and the pulses may be delivered via regular or irregular patterns. Patients may record medication usage and headache intensity, duration, and frequency (e.g., in a diary).

After lead placement (e.g., >7 days, ≥7 days, >10 days, ≥10 days, >14 days, ≥14 days, ≥4 weeks, ≥8 weeks, ≥60 days, ≥90 days, 60-90 days, 30-90 days, >120 days, ≥120 days, 1-4 months, 1-6 months, 3-12 months, ≥12 months), patients may return to the clinic to determine if active stimulation produced pain relief. The stimulus parameters may also be adjusted to obtain comfortable sensations in the appropriate target areas and/or to make adjustments as necessary. Additional visits to verify and better accomplish these aims may be employed.

Lead Removal: Patients may return at a subsequent point. Leads can be removed using gentle traction during a brief (<5 minute) outpatient procedure. The insulating portion of the percutaneous lead may be composed of a non-stick material (e.g., PFA, Teflon), and the lead may be designed to straighten out to facilitate removal when steady gentle traction is applied. Generally speaking, lead removal is not uncomfortable and should not require pain medications or anesthetics. To the extent that the method has been shown to deliver long-term (e.g., at least 3 months pain-free) pain relief, some patients may have the leads removed without the need for additional treatment and/or surgical intervention.

Follow-up: Patients may return after the end of trial stimulation for a safety evaluation of the lead exit site, as well as less regular (e.g., monthly) monitoring to measure the time course for pain relief.

### Case Example

The case was a 68-year-old man with medical history of cervical spondylosis, occipital neuralgia, degeneration of cervical intervertebral disc, and cervical spinal stenosis resulting in chronic headache pain. He previously underwent multiple diagnostic cervical medial branch blocks and cervical medial branch radiofrequency ablations, as well as multiple occipital nerve blocks, none of which provided sufficient and/or sustained pain relief. At the time of the procedure, he was taking diclofenac sodium (a non-steroidal anti-inflammatory drug, or NSAID), acetaminophen-codeine, and citalopram.

At the pre-procedure examination, the subject reported average pain in the region innervated by the occipital nerves (e.g., the posterior head) of 6/10 on a 0-10 pain rating scale where 0 indicates no pain and 10 indicates the worst pain imaginable. The subject also reported a worst pain score of 10/10, and average pain scores in other head regions not innervated by the occipital nerves (e.g., referred pain regions) including 3/10 in the orbital/supraorbital region, 5/10 in the frontotemporal region, and 5/10 in the parietal region. The headache pain was not consistent with migraine symptoms, and the patient reported a score of 1/3 on the ID Migraine Screener, where 2/3 or higher indicates likely presence of a migraine condition. The subject further reported average pain interference of 8.7/10 (averaged across seven domains of daily living including general activity, mood, walking ability, normal work, relations with other people, sleep, and enjoyment of life), which indicates the level to which the headache pain interferes with activities of daily living with 0 indicating no interference and 10 indicating complete interference.

The occipital nerve stimulation therapy involved the implantation of two open coil, percutaneous, temporary leads, one each targeting the left and right greater occipital nerves. Each lead included a single monopolar electrode contact approximately 1.5 cm in length at the distal end of the lead where the most distal 0.5 cm was configured in a bend to serve as an anchor. The two leads were operatively coupled to a dual-channel, external, body-mounted pulse generator. The stimulation protocol consisted of 60 days of stimulation followed by removal of the temporary percutaneous leads. Throughout the stimulation period, multiple aspects of pain, pain interference, function, headache-related disability, and medication usage were periodically measured.

The skin overlying the posterior neck was cleansed and draped with the subject in a prone position and his neck in flexion. Under fluoroscopic guidance, a stimulating needle probe and percutaneous sheath were inserted at the level of the C7 vertebra and advanced from inferior to superior towards the C2/C3 joint in parallel with the articular pillar. The stimulating probe tip was advanced to approximately 1 cm posterior to the C2/C3 joint line and stimulation was tested by increasing the intensity (e.g., the amplitude and/or pulse duration) and the patient was directed to report any new or changed comfortable or uncomfortable sensations evoked by stimulation. At the first testing location on the right side, the stimulation elicited uncomfortable contraction of the muscles in the posterior neck (e.g., semispinalis, obliquus inferior), so the needle was redirected more superiorly and tested again. At the second testing location, the stimulation evoked some comfortable sensations low on the back of the head, but the sensations did not spread to cover the painful region before the increased stimulation intensity caused painful sensations locally near the tip of the needle. After multiple redirections of the needle and testing at various depths (e.g., distances ranging from 0.5-1.5 cm posterior to the C2 lamina and C2/C3 joint), the subject reported comfortable sensations generated by stimulation in the distribution of the occipital nerve when the needle tip was approximately 0.75 cm posterior to the lateral-most portion of the C2 lamina, 1-2 cm superior to the C2/C3 joint. Precise location of the lead was required to balance the objectives of achieving activation of target fibers in the occipital nerve (illustrated by generation of comfortable sensations in the distribution of the occipital nerve) while avoiding activation of non-target fibers such as pain fibers in the occipital nerve, pain fibers in the skin, or motor fibers in the multiple densely packed and highly innervated muscles surrounding the occipital nerve.

The stimulating probe was removed from the percutaneous sheath while the sheath remained in place, and an introducer needle loaded with the stimulating lead was inserted into the sheath to deliver the stimulating lead to the same location where the stimulating probe was located. Stimulation was delivered to the electrode again to ensure proper placement, then the percutaneous sheath and introducer needle were removed together, deploying the lead. Stimulation was then tested a third time after removal of the introducer needle to ensure that the lead's final location still produced the desired comfortable sensations. The second lead was similarly placed on the left side following the same steps, then both lead exit sites were covered with a waterproof bandage such that the top edge of the bandage was inferior to (e.g., lower than) the hairline and the hairline did not interfere with adhesion of the bandage to the skin. The subject's stimulation was programmed to a comfortable intensity, and the subject was issued a wireless controller device to adjust stimulation intensity as needed to maintain comfort.

Over the course of the first week, the subject recorded an average of 20 hours of stimulator usage per day and reported that his average occipital head pain was reduced from 6/10 to 3/10 (a 50% decrease from baseline). Whereas he previously (e.g., at baseline) experienced headache episodes that began upon waking and lasted the entire day until going to bed, he now reported that pain was present upon waking but dissipated within 2-3 hours and the remainder of the day was pain-free. The subject also reported stopping use of pain medications, which he previously only took as needed based on pain intensity. By the second week, the subject reported a further decrease of average occipital head pain to 2/10 (a 67% decrease from baseline), and he further reported reductions in average pain intensity in multiple regions of referred pain: from 3/10 to 2/10 in the orbital/supraorbital region, from 5/10 to 2/10 in the frontotemporal region, and from 5/10 to 3/10 in the parietal region.

This working example describes a subject treated with a dual-lead occipital nerve stimulation system for headache pain. After the first two weeks of electrical stimulation of the occipital nerves, he experienced substantial pain reduction both in the occipital region as well as multiple regions of referred pain, reduced medication usage, and reduced headache episode duration. Additional assessments throughout the 60-day stimulation treatment and follow-up will continue to assess improvements in pain, function, disability, and medication usage.

Although the present embodiments have been illustrated in the accompanying drawings and described in the foregoing detailed description, it is to be understood that the invention is not to be limited to just the embodiments disclosed, and numerous rearrangements, modifications and substitutions are also contemplated. The exemplary embodiment has been described with reference to the preferred embodiments, but further modifications and alterations encompass the preceding detailed description. These modifications and alterations also fall within the scope of the appended claims or the equivalents thereof.

1-60. (canceled)

61. A method to alleviate pain in a head and a neck, the method comprising:
inserting a coiled lead into the neck inferior to the C1 vertebra, wherein the coiled lead: i) is configured to be inserted a length inside a body to facilitate tissue ingrowth along an implanted length of the coiled lead such that the coiled lead is resistant to migration during physiological movement of the head and the neck, ii) is configured with mechanical properties to withstand forces placed upon the coiled lead by stresses, motion, and movement of tissues in an area of occipital nerves;
stimulating at least one occipital nerve through an electrode formed on the coiled lead and operatively coupled to an electrical stimulation device, wherein the electrode is positioned at a therapeutically effective distance from a portion of at least one nerve that innervates the area of the occipital nerves, and outside of an electrically activating distance from non-target nerve fibers in cutaneous tissue, subcutaneous tissue, or muscles proximate to the at least one occipital nerve; activating target fibers in the at least one occipital nerve while preventing activation of non-target fibers in the at least one occipital nerve and preventing activation of the non-target fibers in the cutaneous tissue, subcutaneous tissue, or muscles proximate to the at least one occipital nerve, wherein the activation of the target peripheral nerve fibers produces pain relief in a region of pain in a distribution of the at least one occipital nerve and in one or more region(s) of referred pain outside the distribution of the at least one occipital nerve by modulating central nervous system plasticity associated with the pain, wherein the electrical stimulation comprises a first parameter selected from a group consisting of: frequency, pulse duration, amplitude, duty cycle, pattern of stimulus pulses, polarity, a predetermined number of phases, and waveform shape,
wherein the electrical stimulation comprises a second parameter selected from a group consisting of: frequency, pulse duration, amplitude, duty cycle, pattern of stimulus pulses, polarity, a predetermined number of phases, and waveform shape,
wherein the first parameter is amplitude and the second parameter is pulse duration
wherein an optimal amplitude and pulse duration are selected by increasing the amplitude until discomfort is produced, then decreasing the amplitude and correspondingly increasing the pulse duration to maximize activation of target fibers innervating the region of pain without activating non-target fibers.

62. (canceled)

63. (canceled)

64. (canceled)

65. The method of claim 61, wherein the modulation of central nervous system plasticity associated with chronic pain comprises expansion of non-painful cortical representations and/or contraction of painful cortical representations and/or the functional remapping of body regions in a somatosensory cortex.

66. A system for the relief of pain, the system comprising:
a percutaneous lead configured for insertion into a posterior neck inferior to a nuchal ridge targeting at least one occipital nerve;
at least one electrode formed on the percutaneous lead configured to be positioned at a therapeutically effective distance from the at least one occipital nerve; and
an electrical stimulation device operatively coupled to the percutaneous lead and configured to apply electrical stimulation through the at least one electrode to the at least one occipital nerve to provide relief of pain in a distribution of the at least one occipital nerve and in one or more region(s) of referred pain outside the distribution of the at least one occipital nerve by modulating activity at a point of convergence of the at least one occipital nerve and one or more non-targeted nerve(s) innervating the region of referred pain, wherein the electrical stimulation comprises a first parameter selected from a group consisting of: frequency, pulse duration, amplitude, duty cycle, pattern of stimulus pulses, polarity, a predetermined number of phases, and waveform shape,
wherein the electrical stimulation comprises a second parameter selected from a group consisting of: frequency, pulse duration, amplitude, duty cycle, pattern of stimulus pulses, polarity, a predetermined number of phases, and waveform shape,
wherein the first parameter is amplitude and the second parameter is pulse duration, and
wherein an optimal amplitude and pulse duration are selected by increasing the amplitude until discomfort is produced, then decreasing the amplitude and correspondingly increasing the pulse duration to maximize activation of target fibers innervating the region of pain without activating non-target fibers.

67. The system of claim 66, wherein the percutaneous lead is configured to be inserted a length inside a body to produce tissue ingrowth such that the percutaneous lead is resistant to migration during movement of a head and neck.

68. The system of claim 67, wherein the percutaneous lead is an open coil lead.

69. The system of claim 67, wherein the length to be inserted inside the body is greater than or equal to 4 cm.

70. The system of claim 67, wherein the length is at least three-times greater than a length of the at least one electrode.

71. The system of claim 66, wherein the percutaneous lead comprises one or more anchoring structures.

72. The system of claim 66, wherein the at least one electrode is positioned outside of a central nervous system.

73. The system of claim 66, wherein the percutaneous lead is configured for insertion into a portion of the body that is proximal to the region of pain.

74. The system of claim 73, wherein the electrical stimulation occurs proximal to the region of pain.

75. The system of claim 66, wherein the at least one occipital nerve is selected from a group consisting of: greater occipital nerve, lesser occipital nerve, third occipital nerve, C2 nerve, C3 nerve, C2 medial branch nerve, C3 medial branch nerve, cervical plexus.

76. The system of claim 75, wherein the at least one occipital nerve is selected from a group that comprises at least one distal branch of a selected nerve or nerves.

77. The system of claim 75, wherein the at least one electrode is positioned proximal to a point on the at least one occipital nerve at which one or more nerve fibers branch off from the at least one occipital nerve to innervate distal structures in the region of pain.

78. The system of claim 75, wherein the at least one electrode is positioned along the at least one occipital nerve wherein comfortable sensations are generated only in the region of pain or an area immediately surrounding the region of pain.

79. The system of claim 66, wherein stimulation of the at least one occipital nerve creates comfortable sensations in a distribution of the at least one occipital nerve and does not generate sensations in a distribution of the non-targeted nerve that innervates the region of referred pain.

80. The system of claim 66, wherein the at least one non-targeted nerve is a branch of a trigeminal nerve and the point of convergence is a trigeminocervical complex.

81. The system of claim 80, wherein the region of referred pain is one or more of a parietal, temporal, frontal, frontotemporal, retroorbital, supraorbital, and auricular regions.

82. (canceled)

83. (canceled)

84. (canceled)

85. (canceled)

86. The system of claim 66, wherein the stimulation activates target large diameter fibers in the at least one occipital nerve.

87. The system of claim 86, wherein the stimulation prevents activation of non-target small diameter fibers in the at least one occipital nerve.

88. The system of claim 87, wherein the stimulation prevents activation of non-target fibers in cutaneous tissue, subcutaneous tissue, and muscles proximate to the at least one occipital nerve.

89. The system of claim 88, wherein the stimulation prevents activation of non-target fibers in rotatores, semispinalis cervicis, semispinalis capitis, splenius cervicis, splenius capitis, multifidus, oblique capitis inferior, or trapezius muscles.

90. The system of claim 66, wherein the at least one electrode is formed integrally at the distal end of the lead.

91. The system of claim 66, wherein the at least one electrode is formed integrally on a portion of the lead not at the distal end.

92. The system of claim 66, wherein the percutaneous lead is configured for insertion into the posterior neck inferior to the nuchal ridge targeting at least one occipital nerve at one or more of C2 lamina, C2 lateral articular pillar, C2/C3 joint, C3 lamina, C3 lateral articular pillar, or between the obliquus capitis inferior muscle and semispinalis cervicis muscle.

93. The system of claim 92, wherein an entry site of the percutaneous lead is at least 2 spinal levels inferior to a target location and the percutaneous lead is inserted along a non-intersecting trajectory to achieve the therapeutically effective distance from the at least one occipital nerve.

94. The system of claim 93, wherein the entry site is located inferior to a hairline to facilitate placement of bandaging materials over the entry site.

95. The system of claim 93, wherein the entry site is at the midline and the percutaneous lead is inserted along a non-intersecting trajectory to achieve the therapeutically effective distance from the at least one occipital nerve.

96. A system to alleviate pain in a head and neck, the system comprising:
a coiled lead comprising a diameter facilitating percutaneous insertion into a neck inferior to the C1 vertebra, wherein the coiled lead: i) is configured to be inserted a length inside a body to facilitate tissue ingrowth such that the coiled lead is resistant to migration during movement of the head and neck, ii) is configured with mechanical properties to withstand forces placed upon the coiled lead by stresses, motion, and movement of tissues in an area of occipital nerves preventing mechanical failure;
an electrode formed on the coiled lead, wherein the electrode is positioned at a therapeutically effective distance from a portion of at least one occipital nerve that innervates an occipital region, and outside of an electrically activating distance from non-target nerve fibers in cutaneous tissues, subcutaneous tissues, or muscles surrounding the at least one occipital nerve; and
an electrical stimulation device operatively coupled to the coiled lead and configured to apply electrical stimulation through the electrode to the at least one occipital nerve to activate target peripheral nerve fibers while preventing activation of non-target peripheral nerve fibers in the at least one occipital nerve, wherein activation of target fibers produces relief of headache pain in a region(s) innervated by the at least one occipital nerve and in one or more region(s) of referred pain innervated by one or more non-target nerves by modulating central nervous system plasticity, wherein the coiled lead is configured to be inserted inferior to C1 targeting at least one occipital nerve at one or more of the C2 lamina, C2 lateral articular pillar, C2/C3 joint, C3 lamina, C3 lateral articular pillar, or in one or more muscles in a posterior neck,
wherein an entry site of the coiled lead is at least one spinal level inferior to the target location and wherein the entry site is below C4.

97. The system of claim 96, wherein the coiled lead comprises a diameter configured to be deployed by an introducer needle.

98. The system of claim 97, wherein the introducer needle is greater than or equal to 17 gauge.

99. The system of claim 97, wherein the introducer needle is configured to bend before insertion in the body such that the coiled lead is placed in a location that is not in an unobstructed straight line with an insertion site.

100. The system of claim 97, wherein a maximum radius of curvature of the introducer needle is configured such that a lead path does not turn more than 90 degrees.

101. The system of claim 96, wherein a length of the coiled lead configured to be inserted inside the body is greater than or equal to 4 cm.

102. The system of claim 96, wherein a length of the coiled lead configured to be inserted inside the body is at least three-fold times than a length of the electrode.

103. The system of claim 96, wherein the at least one occipital nerve is selected from a group consisting of: greater occipital nerve, lesser occipital nerve, third occipital nerve, C2 nerve, C3 nerve, C2 medial branch nerve, C3 medial branch nerve, cervical plexus.

104. The system of claim 103, wherein the at least one occipital nerve is selected from a group comprising at least one distal branch of the selected nerve or nerves.

105. The system of claim 96, wherein the at least one non-targeted nerve is the trigeminal nerve or one or more branch(es) of the trigeminal nerve.

106. The system of claim 105, wherein the region of referred pain is one or more of the parietal, temporal, frontal, frontotemporal, retroorbital, supraorbital, and auricular regions.

107. (canceled)

108. The system of claim 66, wherein the muscles include one or more of the rotatores, multifidus, semispinalis cervicis, semispinalis capitis, splenius cervicis, splenius capitis, obliquus capitis inferior, or trapezius.

109. (canceled)

110. (canceled)

111. The system of claim 66, wherein the entry site is located inferior to a hairline.

112. The system of claim 66, wherein the coiled lead is inserted along a non-intersecting trajectory generally from inferior to superior to achieve the therapeutically effective distance from the at least one occipital nerve.

113. The system of claim 66, wherein the entry site is at the midline and the coiled lead is inserted along a non-intersecting trajectory generally from medial to lateral.

114. A system to alleviate pain in a head and neck, the system comprising:
a coiled lead comprising a diameter facilitating percutaneous insertion into a neck inferior to the C1 vertebra, wherein the coiled lead: i) is configured to be inserted a length inside a body to facilitate tissue ingrowth such that the coiled lead is resistant to migration during movement of the head and neck, ii) is configured with mechanical properties to withstand forces placed upon the coiled lead by stresses, motion, and movement of tissues in an area of occipital nerves preventing mechanical failure;
an electrode formed on the coiled lead, wherein the electrode is positioned at a therapeutically effective distance from a portion of at least one occipital nerve that innervates an occipital region, and outside of an electrically activating distance from non-target nerve fibers in cutaneous tissues, subcutaneous tissues, or muscles surrounding the at least one occipital nerve; and
an electrical stimulation device operatively coupled to the coiled lead and configured to apply electrical stimulation through the electrode to the at least one occipital nerve to activate target peripheral nerve fibers while preventing activation of non-target peripheral nerve fibers in the at least one occipital nerve, wherein activation of target fibers produces relief of headache pain in a region(s) innervated by the at least one occipital nerve and in one or more region(s) of referred pain innervated by one or more non-target nerves by modulating central nervous system plasticity, wherein the therapeutically effective distance is a remote location that enables activation of target fibers in the at least one occipital nerve while avoiding non-target fiber activation in the at least one occipital nerve,
wherein the target fibers in the at least one occipital nerve comprise large diameter sensory fiber and the activation of the target fibers generates comfortable sensations in a distribution of the at least one occipital nerve, wherein the comfortable sensations are generated in the region of pain and the comfortable sensations are not generated in the region of referred pain.

115. The system of claim 114, wherein the therapeutically effective distance is 0.5-3.0 cm.

116. (canceled)

117. (canceled)

118. (canceled)

119. (canceled)

120. The system of claim 114, wherein the non-target fibers in the at least one occipital nerve comprise small diameter fibers.

121. The system of claim 96, wherein the non-target nerve fibers include small diameter fibers in cutaneous tissue layers, subcutaneous tissue layers, or muscle tissue in the vicinity of the at least one occipital nerve and the electrode.

122. The system of claim 96, wherein the non-target fibers comprise motor fibers in one or more muscle(s) in a vicinity of the at least one occipital nerve and the electrode.

123. The system of claim 122, wherein the muscles comprise one or more of the rotatores, multifidus, semispinalis cervicis, semispinalis capitis, splenius cervicis, splenius capitis, obliquus capitis inferior, or trapezius.

124. The system of claim 96, wherein the electrical stimulation device comprises an external, body-worn pulse generator.

125. The system of claim 96, wherein the electrical stimulation device comprises an implanted pulse generator.

126. A system to alleviate pain in a head and neck, the system comprising:
a coiled lead comprising a diameter facilitating percutaneous insertion into a neck inferior to the C1 vertebra, wherein the coiled lead: i) is configured to be inserted a length inside a body to facilitate tissue ingrowth such that the coiled lead is resistant to migration during movement of the head and neck, ii) is configured with mechanical properties to withstand forces placed upon the coiled lead by stresses, motion, and movement of tissues in an area of occipital nerves preventing mechanical failure;
an electrode formed on the coiled lead, wherein the electrode is positioned at a therapeutically effective distance from a portion of at least one occipital nerve that innervates an occipital region, and outside of an electrically activating distance from non-target nerve fibers in cutaneous tissues, subcutaneous tissues, or muscles surrounding the at least one occipital nerve; and
an electrical stimulation device operatively coupled to the coiled lead and configured to apply electrical stimulation through the electrode to the at least one occipital nerve to activate target peripheral nerve fibers while preventing activation of non-target peripheral nerve fibers in the at least one occipital nerve, wherein activation of target fibers produces relief of headache pain in a region(s) innervated by the at least one occipital nerve and in one or more region(s) of referred pain innervated by one or more non-target nerves by modulating central nervous system plasticity, wherein the electrical stimulation comprises a first parameter selected from a group consisting of: frequency, pulse duration, amplitude, duty cycle, pattern of stimulus pulses, polarity, a predetermined number of phases, and waveform shape,
wherein the electrical stimulation comprises a second parameter selected from a group consisting of: frequency, pulse duration, amplitude, duty cycle, pattern of stimulus pulses, polarity, a predetermined number of phases, and waveform shape,
wherein the first parameter is amplitude and the second parameter is pulse duration, and
wherein an optimal amplitude and pulse duration are selected by increasing the amplitude until discomfort is produced, then decreasing the amplitude and correspondingly increasing the pulse duration to activate target fibers innervating the region of pain without activating non-target fibers.

127. (canceled)

128. (canceled)

129. (canceled)

130. The system of claim 126, wherein the modulation of central nervous system plasticity associated with chronic pain comprises an expansion of non-painful cortical representations and/or contraction of painful cortical representations and/or functional remapping of body regions in a somatosensory cortex.
